(19) United States
(12) Patent Application Publication
(10) Pub. No.: US 2005/0215548 A1
(43) Pub. Date: Sep. 29, 2005

(54) Bisarylsulfonamide compounds and their use in cancer therapy

(75) Inventors: Shudong Wang, Angus (GB); Darren Gibson, Dundee (GB); Kenneth Duncan, Stirlingshire (GB); Kevin Bailey, Derbyshire (GB); Mark Thomas, Dundee (GB); David MacCallum, By Forfar (GB); Daniella I. Zheleva, Fife (GB); Nicholas John Turner, Edinburgh (GB); Peter Martin Fischer, Arbroath (GB)

Correspondence Address: LAHIVE & COCKFIELD, LLP., 28 STATE STREET, BOSTON, MA, 02109, US

(73) Assignee: Cyclacel Limited, Dundee (GB)

(21) Appl. No.: 10/988,388

(22) Filed: Nov. 12, 2004

Related U.S. Application Data
(63) Continuation of application No. PCT/GB03/02923, filed on Jul. 7, 2003.

(30) Foreign Application Priority Data
Jul. 5, 2002 (GB) 0215650.3

(51) Int. Cl. A61K 31/5377; A61K 31/454; A61K 31/4439; A61K 31/43

(52) U.S. Cl. 514/235.2; 514/338; 514/366; 514/414; 514/322; 514/323; 514/406

(57) ABSTRACT

The present invention relates to the use of bisarylsulfonamide compounds of formula I 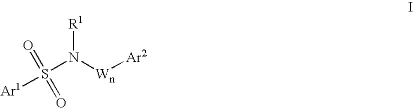 wherein
W is a C₁₋₅ branched or unbranched alkyl group or a C₂₋₅ alkenyl group; n is 0 or 1;
R¹ is H, a C₁₋₈ branched or unbranched alkyl group, a C₂₋₈ alkenyl group, or an aryl or aralkyl group;
Ar¹ is a substituted thienyl, furyl, pyrrolyl, imidazothiazolyl, thiazolyl, pyridyl or phenyl group; and
Ar² is a substituted phenyl, indolyl or benzoimidazolyl group; in the preparation of a medicament for treating proliferative disorders. Further aspects of the invention relate to compounds of formula I, pharmaceutical compositions thereof, and an assay for determining binding to HDM2.

### RELATED APPLICATIONS

This application is a continuation of PCT/GB2003/002923, filed on Jul. 7, 2003, which claims priority to GB 0215650.3, filed on Jul. 5, 2002. The entire contents of each of these applications are hereby incorporated herein by reference.

### FIELD OF INVENTION

The present invention relates to bisarylsulfonamide compounds. In particular, the invention relates to bisarylsulfonamide compounds that are capable of binding to the oncoprotein HDM2 and modulating the HDM2-dependent regulation of the tumour suppressor p53 and/or E2F transcription factors in living cells. Further aspects of the invention relate to pharmaceutical preparations comprising such compounds, and the use thereof, e.g. in the therapeutic treatment of humans or animals.

### BACKGROUND

The MDM2 oncogene was first cloned as an amplified gene on a murine double-minute chromosome; the encoded proto-oncoprotein is designated MDM2 and the human equivalent protein is known as HDM2 [Zhang, Wang H; Curr. Pharmaceut. Design 2000; 6: 393-416]. The connection between HDM2 and human cancer is well established. Thus HDM2 is overexpressed in a variety of tumours due to gene amplification or increased transcription or translation [Momand J, Jung D, Wilczynski S, Niland J; Nucleic Acids Res. 1998; 26: 3453-3459].

Inactivation of the p53 tumour suppressor protein is a frequent event in human neoplasia [Lane D P, Lain S; Trends Molec. Med. 2002; 8: S38-S42.]. Apart from inactivation due to mutational defects, this may result, for example, from the binding of HDM2. Interaction of HDM2 with p53 in vitro or in vivo results in inhibition of p53-mediated transactivation. Formation of HDM2/p53 complexes favours nucleoplasmic transformation because of the loss of the p53 tumour suppressor effects upon complexation. HDM2 forms a negative autoregulatory loop with p53 by binding to its N-terminal activation domain thereby inhibiting the functions of p53 and promoting the proteolytic degradation of p53. Interference with this regulatory loop can be used to increase the concentration of active p53 in cells.

The HDM2-binding site on p53 was identified with the aid of a set of overlapping synthetic peptides [Picksley S M, Voijtesek B, Sparks A, Lane D P; Oncogene 1994; 9: 2523-2529] and was mapped to the sequence ¹⁸TFSDLW²³ (SEQ ID No.: 1). Although longer peptides encompassing this sequence were potent inhibitors of p53/HDM2 complex formation, the hexapeptide p53(18-23) itself had little affinity [Böttger A, Böttger V, Garcia-Echeverria C, Chène P, Hochkeppel H K, Sampson W, Ang K, Howard S F, Picksley S M, Lane D P; J. Molec. Biol. 1997; 269: 744-756].

Screening of phage-displayed peptide libraries also revealed sequences containing the HDM2-binding motif [Böttger V, Böttger A, Howard S F, Picksley S M, Chene P, Garcia-Echeverria C, Hochkeppel H K, Lane D P; Oncogene 1996; 13: 2141-2147]. Here the starting 12 mer peptide MPRFMDYWEGLN (SEQ ID NO.: 2) had sub-micromolar affinity and was 28-fold more potent than the corresponding wild-type p53-derived peptide ¹⁶QETFSDLWKLLF²⁷ (SEQ ID NO: 3). Substitution and truncation studies revealed that the 8 mer peptide FMDYWEGL (SEQ ID NO.: 4) was the minimal active sequence retaining micromolar affinity for HDM2 [Böttger A et al, ibid]. Based on the known binding mode of the corresponding p53 sequence [Kussie P H, Gorina S, Marechal V, Elenbaas B, Moreau J, Levine A J, Pavletich N P; Science 1996; 274: 948-953], the helical structure of this peptide was stabilised by introduction of α,α-disubstituted amino acid residues α-aminoisobutyric (Aib) acid and 1-aminocyclopropanecarboxylic acid (Ac₃c) in place of the Asp and Gly residues, respectively. Molecular modelling suggested proximity of the Tyr side chain to the ε-amino group of the HDM2 Lys⁹⁴ residue and a phosphonomethylphenylalanine (Pmp) residue was used to replace Tyr. The resulting peptide was about 7-fold more potent, suggesting that the hypothetical stabilising salt bridge between the phosphonate and amino groups was in fact operating. Finally, inspection of the binding pocket for Trp²³ showed incomplete occupancy, suggesting substituents at the indole 6-position would improve binding. This was the case and substantial potency gain was obtained. Thus starting with the wild-type p53 12 mer sequence the affinity was increased by >1,700-fold.

The crystal structure of the p53/HDM2 complex, as well as the peptide optimisation work discussed above, show that the main contacts between p53 and the hydrophobic cleft in HDM2 involve only three p53 residues (Phe¹⁹, Trp²³, and Leu²⁶). The molecular mass of the three side chains in question amounts to ca. 300 Da, suggesting that modulation of the p53/HDM2 protein-protein interaction with non-peptidic small molecules may in fact be feasible. The validity of this hypothesis has already been confirmed to some extent.

Screening microbial extracts for the presence of inhibitors of the p53/HDM2 interaction, a fungal metabolite known as chlorofusin was identified as a micromolar inhibitor [Duncan S J, Grueschow S, Williams D H, McNicholas C, Purewal R, Hajek M, Gerlitz M, Martin S, Wrigley S K, Moore M; J. Am. Chem. Soc. 2001; 123: 554-560]. Chalcones (1,3-diphenyl-2-propen-1-ones) have long been known to possess anti-tumour effects [De Vincenzo R, Ferlini C, Distefano M, Gaggini C, Riva A, Bombardelli E, Morazzoni P, Valenti P, Belluti F, Ranelletti F O, Mancuso S, Scambia G; Cancer Chemotherapy Pharmacol. 2000; 46: 305-312]. Certain chalcone derivatives, in particular a compound referred to as “B-1” {4-[3-(3,4-dichloro-phenyl)-acryloyl]-phenoxy}-acetic acid, and some of its analogues, were shown to inhibit the p53/HDM2 complex with high micromolar affinity [Stoll R, Renner C, Hansen S, Palme S, Klein C, Belling A, Zeslawski W, Kamionka M, Rehm T, Muehlhahn P, Schumacher R, Hesse F, Kaluza B, Voelter W, Engh R A, Holak T A; Biochemistry 2001; 40: 336-344]. Using multidimensional NMR techniques, evidence for direct binding of the chalcone derivatives to the Trp²³-binding pocket sub-site of the p53 binding cleft of HDM2 was presented.

Finally, peptidomimetic design starting from p53-derived HDM2-binding peptides led to acyltryptophanylpiperazides, p53/HDM2 antagonists with low micromolar affinity [Luke R W A, Hudson K, Hayward C F, Fielding C, Cotton R, Best R, Giles M B, Veldman M H, Griffiths L A, Jewsbury P J, Breeze A L, Embrey K J; Proc. Amer. Assoc. Cancer Res. 1999; 40: #4099; Luke R W A, Jewsbury P J, Cotton R; PCT Int. Patent Appl. Publ. WO 00/15657; Zeneca Ltd., UK, 2000].

The present invention seeks to provide therapeutic agents that are useful in the treatment of cancer and other proliferative disorders.

### STATEMENT OF INVENTION

A first aspect of the invention relates to the use of a compound of formula I, 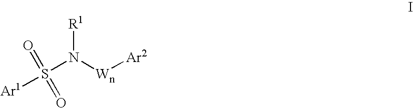
wherein
W is a C₁₋₅ branched or unbranched alkylene group or a C₂₋₅ alkenylene group;
n is 0 or 1;
R¹ is H, a C₁₋₈ branched or unbranched alkyl group, a C₂₋₈ alkenyl group, or an aryl or aralkyl group, each of which may be optionally substituted by one or more halogen or CF₃ groups;
Ar¹ is 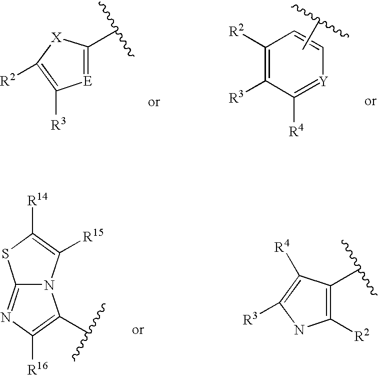
   wherein
   X is S, O, NH or NR′ where R′ is a C₁₋₃ alkyl group;
   Y is CH or N;
   E is N or CR⁴;
   R², R³, R⁴, and R¹⁴⁻¹⁶ are each independently (A)ₚB, wherein A is C₁₋₃ alkyl, p is 0 or 1, and B is H, halogen, C₁₋₅ alkyl, NO₂, OH, NH₂, NHR^{a}, NR^{b}R^{c}, SO₃H, SO₂NH₂, NHAr^{a}, SO₂NHAr^{b}, SO₂NHR^{d}, SO₂Ar^{c}, SO₂R^{e}, CF₃, CN, COOH, COOR^{f}, CONH₂, COONHAr^{d}, CONHR^{g}, COAr^{e}, COR^{h}, S(CO)R^{s}, OR^{t}, OAr^{f}, an alicyclic group optionally containing one or more heteroatoms, optionally substituted by one or more OH, COR^{u}, halogen or CF₃ groups, or a heteroaryl group optionally substituted by one or more C₁₋₅ alkyl, halogen, SRⁱ or CF₃ groups; or
   R² and R³ are linked to form a saturated or unsaturated ring system, optionally containing one or more heteroatoms, and optionally substituted by one or more halogen, OH or CF₃ groups;
   Ar^{a-f} are each independently aryl groups optionally substituted by one or more C₁₋₅ alkyl, halogen or CF₃ groups;
   R^{a-i}, R^{s}, R^{t} and R^{u} are each independently C₁₋₅ alkyl groups optionally substituted by one or more alkoxy, halogen or CF₃ groups;
   and with the proviso that at least one of R², R³ and R⁴ is other than H;
      Ar² is 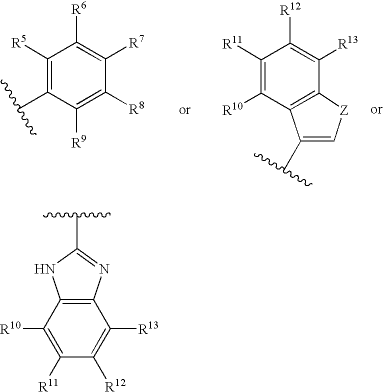
   wherein
   Z is S, O, NH or NR″ where R″ is C₁₋₃ alkyl;
   R⁵, R⁶, R⁷, R⁸ and R⁹ are each independently (L)_{q}M wherein L is C₁₋₃ alkyl, q is 0 or 1, M is H, C₁₋₅ alkyl, halogen, NO₂, OH, NH₂, NHAr^{g}, NHR^{j}, NR^{k}R^{l}, SO₃H, SO₂NH₂, SO₂NHAr^{h}, SO₂NHR^{m}, SO₂Arⁱ, SO₂Rⁿ, CF₃, CN, COOH, COOR^{p}, CONH₂, CONHAr^{j}, CONHR^{q}, OR^{v}, COAr^{k} or COR^{r};
   R^{j-r}, R^{v} are each independently C₁₋₅ alkyl groups;
   Ar^{g-k} are each independently aryl groups;
   and with the proviso that at least one of the substituents R⁵, R⁶, R⁷, R⁸ and R⁹ is other than H;
   R¹⁰, R¹¹, R¹² and R¹³ are each independently H, C₁₋₅ alkyl, halogen, NO₂, OH, NH₂ or CF₃;
      in the preparation of a medicament for treating a proliferative disorder.

A second aspect of the invention relates to the use of a compound as defined hereinabove for treating a proliferative disorder.

A third aspect of the invention relates to compounds of formula Ia, Ib, Ic and Id as outlined in more detail below.

A fourth aspect of the invention relates to a pharmaceutical composition comprising a compound according to the invention admixed with one or more pharmaceutically acceptable diluents, excipients or carriers.

A fifth aspect of the invention relates to the use of a compound of the invention in an assay for determining binding to HDM2.

A sixth aspect of the invention provides a method of detecting the binding of a ligand to HDM2, said method comprising the steps of:

(i) contacting a ligand with HDM2 in the presence of a p53-derived peptide; and

(ii) detecting any change in the interaction between HDM2 and said p53-derived peptide;

and wherein said ligand is a compound according to the invention.

A seventh aspect of the invention relates to a combination comprising at least one compound of the invention and at least one cytotoxic agent.

An eighth aspect relates to a pharmaceutical composition comprising at least one compound of the invention, and one or more cytotoxic agents, admixed with a pharmaceutically acceptable diluent, excipient or carrier.

A ninth aspect of the invention provides a method of treating a proliferative disorder, said method comprising administering to a subject at least one compound of the invention, consecutively, simultaneously or sequentially with one or more other cytotoxic agents.

A tenth aspect of the invention relates to a method of treating a proliferative disorder, said method comprising administering to a subject at least one compound of the invention consecutively, simultaneously or sequentially with radiotherapy.

### BRIEF DESCRIPTION OF THE DRAWINGS

FIG. 1 shows the effect of compound 2 or DNA damage inducing agents on the levels of key proteins in the HDM2 pathway and cell morphology in a range of cell lines.

FIG. 2 shows the effect of compound 49 on AGS, SJSA1 and H1299 cells.

FIG. 3 shows the effect of compound 2 on the morphology and cell viability of MCF7 and H1299 cells.

FIG. 4 shows the effect of compounds 68 and 69 on MCF7 cells and H 1299 cells.

FIG. 5 shows the effect of compound 2 and cisplatinum on the cell cycle distribution of MCF7 and H1299 cells.

FIG. 6 shows the effect of compound 2 on the caspase activation in AGS and H1299 cells.

FIG. 7 shows the effect of HDM2 and HDMX siRNA on the levels of p53 and E2F-1 in MCF7 cells.

### DETAILED DESCRIPTION

As mentioned, a first aspect of the invention relates to the use of a compound of formula I as defined hereinabove in the preparation of a medicament form treating proliferative disorders.

As used herein the phrase “preparation of a medicament” includes the use of a compound of the invention directly as the medicament in addition to its use in a screening programme for the identification of further agents or in any stage of the manufacture of such a medicament.

Such a screening programme may for example include an assay for determining the binding to HDM2 and determining whether a candidate substance is capable of mimicking the activity of a compound of formula I.

The present invention relates to bisarylsulfonamides as therapeutic agents in the treatment of proliferative disorders.

In one preferred embodiment, Ar¹ is 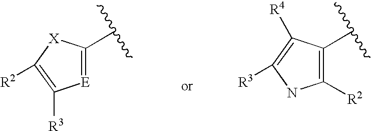
and Ar² is

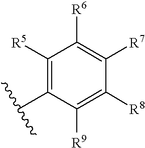

In an alternative preferred embodiment, Ar¹ is 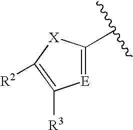
and Ar² is

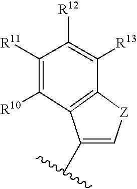

In another preferred embodiment, Ar¹ is 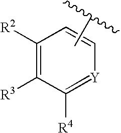
and Ar² is

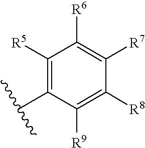

In another preferred embodiment, Ar¹ is 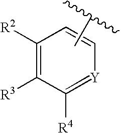
and Ar² is

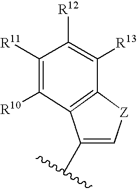

In yet another preferred embodiment, Ar¹ is 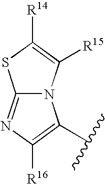
and Ar² is

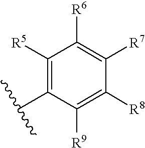

In another preferred embodiment, Ar¹ is 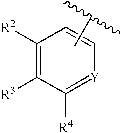
and Ar² is

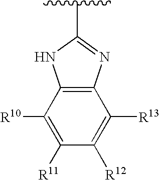

For all of the above embodiments, preferably,
R², R³ and R⁴ are each independently (A)ₚB, wherein A is C₁₋₅ alkyl, p is 0 or 1, and B is H, F, Cl, Br, I, C₁₋₅ alkyl, NO₂, OH, NH₂, NHR^{a}, NR^{b}R^{c}, SO₃H, SO₂NH₂, NHPh, SO₂NHAr^{b}, SO₂NHR^{d}, SO₂Ph, SO₂R^{e}, CF₃, CN, COOH, COOR^{f}, CONH₂, COONHPh, CONHR^{g}, S(CO)R^{s}, OR^{t}, OAr^{f}, COPh, COR^{h}, a morpholino, piperazino or piperidino group each of which may be optionally substituted by one or more OH or COR^{u} groups, or a heteroaryl group selected from pyridyl, pyrimidyl, oxazolyl, thiazolyl and pyrazolyl, each of which may be optionally substituted by one or more C₁₋₅ alkyl, halogen, SRⁱ or CF₃ groups, or R² and R³ together form a saturated 6-membered ring or an unsaturated 5-membered ring, each of which optionally contain one or more heteroatoms; and
R⁵, R⁶, R⁷, R⁸, and R⁹ are each independently (L)_{q}M wherein L is C₁₋₅ alkyl, q is 0 or 1, M is H, C₁₋₅ alkyl, halogen, NO₂, OH, NH₂, NHPh, NHR^{j}, NR^{k}R^{l}, SO₃H, SO₂NH₂, SO₂NHPh, SO₂NHR^{m}, SO₂Ph, SO₂Rⁿ, CF₃, CN, COOH, COOR^{p}, CONH₂, CONHPh, CONHR^{q}, OR^{v}, COPh or COR^{r}.

More preferably, R², R³ and R⁴ are each independently (A)ₚB, wherein A is C₁₋₅ alkyl, p is 0 or 1, and B is H, F, Cl, Br, I, C₁₋₅ alkyl, NO₂, OH, NH₂, NHR^{a}, NR^{b}R^{c}, SO₃H, SO₂NH₂, NHPh, SO₂NHPh, SO₂NHR^{d}, SO₂Ph, SO₂R^{e}, CF₃, CN, COOH, COOR^{f}, CONH₂, COONHPh, CONHR^{g}, S(CO)R^{s}, OR^{t}, OAr^{f}, COPh, COR^{h}, pyridyl, pyrimidyl, 2-methylsulfanylpyrimid-5-yl, oxazol-2-yl, thiazol-2-yl, 1-methyl-5-trifluoromethyl-1H-pyrazol-4-yl, morpholin-4-yl, 4-acetyl-piperazin-1-yl, 3-hydroxy-piperidin-1-yl, or R² and R³ together form
—OCH₂CH₂O—
—N—S—N— or
a phenyl group optionally substituted by one or more halogens.

More preferably still, R², R³ and R⁴ are each independently H, halogen, NO₂, SO₂Ph, S(CO)Me, COOH, COOEt, OPh, OMe, NHCH₂CH₂OMe, 1-methyl-5-trifluoromethyl-1H-pyrazol-4-yl, 2-methylsulfanyl pyrimid-5-yl, N-(4-fluorophenyl)sulfonamido, N-(4-trifluoromethylphenyl)-sulfonamido, oxazol-2-yl, C₁₋₅ alkyl, NH₂, morpholin-4-yl, 4-acetyl-piperazin-1-yl, 3-hydroxy-piperidin-1-yl, or R² and R³ together form
OCH₂CH₂O—
—N—S—N— or
a phenyl group optionally substituted by one or more halogens.

In one particularly preferred embodiment, Ar¹ is 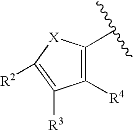
X is S or N;
R², R³ and R⁴ are each independently C₁₋₅ alkyl, S(CO)Me, COOH, NHCH₂CH₂OMe, COOEt, H, halogen, NO₂, SO₂Ph, SO₂NH-(4-chlorophenyl), 1-methyl-5-trifluoromethyl-1H-pyrazol-4-yl, morpholin-4-yl, 2-methylsulfanylpyrimid-5-yl, N-(4-fluorophenyl)sulfonamido, N-(4-trifluoro-methylphenyl)-sulfonamido, 3-hydroxy-piperidin-1-yl, pyridin-2-yl; or R² and R³ form a phenyl group optionally substituted by one or more halogens.

More preferably, for this embodiment,
R² is halogen, SO₂Ph, NO₂, Et, SOMe, morpholin-4-yl, NHCH₂CH₂OMe, 3-hydroxy-piperidin-1-yl, 1-methyl-5-trifluoromethyl-1H-pyrazol-4-yl or 2-methylsulfanyl-pyrimid-5-yl;
R³ is halogen, SO₂NH-(4-chlorophenyl), H, NO₂, N-(4-fluorophenyl)sulfonamido or N-(4-trifluoro methylphenyl)-sulfonamido; and
R⁴ is H.

In another particularly preferred embodiment, Ar¹ is 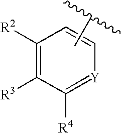
Y is CH or N; and
R², R³ and R⁴ are each independently H, OH, COOH, CF₃, OPh, OMe, NO₂, 4-acetyl-piperazin-1-yl, NH₂, halogen, pyrazol-1-yl, oxazol-2-yl or C₁₋₅ alkyl, or R² and R³ together form —OCH₂CH₂O— or —N—S—N—.

More preferably, for this embodiment,
when Y is CH
   R² is H, NO₂ or Cl;
   R³ is NO₂, NH₂, Cl, CF₃, COOH, 4-acetyl-piperazin-1-yl; and
   R⁴ is H, Cl, oxazol-2-yl, OH, NO₂, NH₂, OMe or Me; or
when Y is N
   R² is H;
   R³ is Br;
   R⁴ is Cl or OPh.

In another preferred embodiment of the invention, Ar¹ is 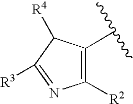
R² and R⁴ are C₁₋₅ alkyl, and R³ is COOH or COOEt.

In one preferred embodiment,
R⁵, R⁶, R⁷, R⁸ and R⁹ are each independently H, halogen, OMe, NO₂, C₁₋₅ alkyl, CF₃ or OH; and
R¹⁰, R¹¹, R¹² and R¹³ are all H.

More preferably still,
R⁵ is H, C₁₋₅ alkyl, or halogen;
R⁶ is H, halogen, NO₂, or CF₃;
R⁷ is H, halogen, OMe, NO₂, OH or CF₃;
R⁸ is H, halogen or CF₃;
R⁹ is H.

In one preferred embodiment of the invention,
W is CH₂, CH₂CH₂ or CH(CH₃)CH₂; and
R¹ is H, CH₂Ph, CH₂CH(Me)₂, 3-(trifluoromethyl)benzyl, or Me.

In an especially preferred embodiment of the first aspect of the invention, the compound of formula I is selected from the following:
5-Chloro-4-nitrothiophene-2-sulfonic acid (3-trifluoromethylphenyl)amide [1];
5-Chloro-4-nitrothiophene-2-sulfonic acid (4-chlorophenyl)-amide [2];
5-Chloro-4-nitrothiophene-2-sulfonic acid (4-fluorophenyl)amide [3];
4-Bromo-5-chlorothiophene-2-sulfonic acid (4-fluorophenyl)amide [4];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
4,5-Dibromothiophene-2-sulfonic acid (3,5-bis-trifluoromethylphenyl)amide [8];
5-Chlorothiophene-2-sulfonic acid (4-trifluoromethylphenyl)amide [9];
5-Chlorothiophene-2-sulfonic acid (4-chlorophenyl)amide [10];
5-Chlorothiophene-2-sulfonic acid (3,5-bis-trifluoromethylphenyl)amide [11];
5-(2-Methylsulfanyl-pyrimidin-5-yl)-thiophene-2-sulfonic acid (3,5-dichlorophenyl)-amide [12];
4-Oxazol-2-yl-N-(4-trifluoromethylphenyl)benzenesulfonamide [13];
N-(3,5-Bis-trifluoromethylphenyl)-4-oxazol-2-yl-benzenesulfonamide [14];
4-Bromo-5-chlorothiophene-2-sulfonic acid (4-trifluoromethylphenyl)amide [15];
5-Bromothiophene-2-sulfonic acid (4-chlorophenyl)amide [16];
5-Bromothiophene-2-sulfonic acid (3,5-dichlorophenyl)amide [17];
5-Bromothiophene-2-sulfonic acid (3,5-bis-trifluoromethylphenyl)amide [18];
N-(4-Chlorophenyl)-3-nitrobenzenesulfonamide [19];
3-Nitro-N-(4-trifluoromethylphenyl)benzenesulfonamide [20];
N-(3,5-Bis-trifluoromethylphenyl)-3-nitrobenzenesulfonamide [21];
N-(2,4-Dichlorophenyl)-3-nitrobenzenesulfonamide [22];
5-Benzenesulfonylthiophene-2-sulfonic acid (4-trifluoromethylphenyl)-amide [23];
5-Benzenesulfonylthiophene-2-sulfonic acid (4-chlorophenyl)amide [24];
5-Benzenesulfonylthiophene-2-sulfonic acid (3,5-dichlorophenyl)amide [25];
5-Chlorothiophene-2-sulfonic acid (3,4-dichlorophenyl)amide [26];
4,5-Dibromothiophene-2-sulfonic acid (3-trifluoromethylphenyl)amide [27];
4,5-Dibromothiophene-2-sulfonic acid (3,4-dichlorophenyl)amide [28];
N-(3,5-Bis-trifluoromethylphenyl)-4-chloro-3-nitrobenzenesulfonamide [29];
4-Chloro-N-(3,4-dichlorophenyl)-3-nitrobenzenesulfonamide [30];
5-(1-Methyl-5-trifluoromethyl-1H-pyrazol-4-yl)-thiophene-2-sulfonic acid (4-trifluoromethylphenyl)-amide [31];
5-Chlorothiophene-2,4-disulfonic acid bis-[(4-fluorophenyl)-amide] [32];
5-Chlorothiophene-2,4-disulfonic acid bis-[(4-trifluoro-methyl-phenyl)-amide] [33];
4-Methyl-3-nitro-N-(4-trifluoromethylphenyl)benzenesulfonamide [34];
4-Chloro-3-nitro-N-(4-trifluoromethylphenyl)benzenesulfonamide [35];
3-Amino-4-methyl-N-(4-trifluoromethyl-phenyl)benzenesulfonamide [36];
N-(4-Chlorophenyl)-4-methyl-3-nitrobenzenesulfonamide [37];
4-Chloro-N-(4-chlorophenyl)-3-nitro-benzenesulfonamide [38];
5-Chloro-4-nitrothiophene-2-sulfonic acid (3,5-dichlorophenyl)-amide [39];
5-Chloro-4-nitrothiophene-2-sulfonic acid (3,5-difluorophenyl)-amide [40];
5-Bromo-6-chloropyridine-3-sulfonic acid (4-trifluoromethylphenyl)amide [41];
5-Bromo-6-chloropyridine-3-sulfonic acid (3,5-bis-trifluoromethylphenyl)amide [42];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethyl-phenyl)-amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
4-Chloro-N-(3,5-dichlorophenyl)-3-nitro-benzenesulfonamide [47];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid methyl-(4-trifluoromethylphenyl)amide [50];
5-Chloro-4-nitrothiophene-2-sulfonic acid (4-chloro-phenyl)-methylamide [51]; and
5-Chloro-4-nitrothiophene-2-sulfonic acid methyl-(4-trifluoromethylbenzyl)amide [52];
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (4-chloro-phenyl)-isobutyl-amide [58];
5-Chloro-4-nitro-thiophene-2-sulfonic acid (1H-benzo-imidazol-2-yl)-amide [59];
5-Chloro-4-nitro-thiophene-2-sulfonic acid [2-(6-chloro-1H-indol-3-yl)-ethyl]-amide [60];
5-Chloro-4-nitro-thiophene-2-sulfonic acid (4-methoxy-phenyl)-amide [61];
5-Chloro-4-nitro-thiophene-2-sulfonic acid p-tolylamide [63];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
5-Nitro-thiophene-2-sulfonic acid (4-chloro-phenyl)-amide [68];
4-Nitro-thiophene-2-sulfonic acid (4-chloro-phenyl)-amide [69];
5-Chloro-thiophene-2,4-disulfonic acid bis-[(4-chloro-phenyl)-amide] [70];
5-Ethyl-4-nitro-thiophene-2-sulfonic acid (4-chloro-phenyl)-amide [71];
Thioacetic acid S-[5-(4-chloro-phenylsulfamoyl)-3-nitro-thiophen-2-yl] ester [72];
5-Methyl-4-nitro-thiophene-2-sulfonic acid (4-chloro-phenyl)-amide [73];
5-Methyl-thiophene-2,4-disulfonic acid bis-[(4-chloro-phenyl)-amide] [74];
5-Chloro-4-nitro-thiophene-2-sulfonic acid (3-trifluoro-methyl-benzyl)-(4-trifluoromethyl-benzyl)-amide [75];
4-Nitro-thiophene-2-sulfonic acid (4-trifluoromethyl-phenyl)-amide [76];
4-Nitro-thiophene-2-sulfonic acid [2-(1H-indol-3-yl)-ethyl]-amide [77];
5-(1-Methyl-5-tri-fluoromethyl-1H-pyrazol-3-yl)-thiophene-2-sulfonic acid (3,5-bis-tri-fluoromethyl-phenyl)-(3-trifluoro-methyl-benzyl)-amide [78];
5-Morpholin-4-yl-4-nitro-thiophene-2-sulfonic acid (4-chloro-phenyl)-amide [79];
5-(2-Methoxy-ethylamino)-4-nitro-thiophene-2-sulfonic acid (4-chloro-phenyl)-amide [80];
4-Chloro-N-[2-(5-chloro-1H-indol-3-yl)-ethyl]-3-nitro-benzenesulfonamide [81];
N-[2-(5-Chloro-1H-indol-3-yl)-ethyl]-4-methyl-3-nitro-benzenesulfonamide [82];
N-(1H-Benzoimidazol-2-yl)-4-chloro-3-nitro-benzenesulfonamide [83];
6-Chloro-imidazo[2,1-b]thiazole-5-sulfonic acid (3,5-bis-trifluoro-methyl-phenyl)-amide [84];
2,3-Dihydro-benzo[1,4]dioxine-6-sulfonic acid (4-chloro-phenyl)-amide [85];
2,3-Dihydro-benzo[1,4]dioxine-6-sulfonic acid (3,5-bis-trifluoromethyl-phenyl)-amide [86];
6-Phenoxy-pyridine-3-sulfonic acid (4-chloro-phenyl)-amide [87];
5-Chloro-3-methyl-benzo[b]thiophene-2-sulfonic acid (4-chloro-3-nitro-phenyl)-amide [88];
N-(3,5-Bis-trifluoromethyl-phenyl)-4-pyrazol-1-yl-benzenesulfonamide [89];
4-(4-Chloro-phenylsulfamoyl)-3,5-dimethyl-1H-pyrrole-2-carboxylic acid ethyl ester [90];
4-(3,5-Bis-trifluoromethyl-phenylsulfamoyl)-3,5-dimethyl-1H-pyrrole-2-carboxylic acid [91];
4-(4-Chloro-phenylsulfamoyl)-3,5-dimethyl-1H-pyrrole-2-carboxylic acid [92];
2-(4-Chloro-phenylsulfamoyl)-4-methyl-thiazole-5-carboxylic acid ethyl ester [93];
3,5-Dichloro-N-(4-chloro-phenyl)-4-hydroxy-benzenesulfonamide [94];
N-(3,5-Bis-trifluoromethyl-phenyl)-3,5-dichloro-4-hydroxy-benzenesulfonamide [95];
3,5-Dichloro-4-hydroxy-N-(4-trifluoromethyl-phenyl)-benzenesulfonamide [96];
N-(4-Chloro-phenyl)-4-nitro-benzene-sulfonamide [97];
N-(3,5-Bis-trifluoromethyl-phenyl)-4-nitro-benzenesulfonamide [98];
4-Amino-N-(3,5-bis-trifluoromethyl-phenyl)-3-chloro-benzenesulfonamide [99];
3-Nitro-N-(4-trifluoromethyl-phenyl)-benzenesulfonamide [100];
3,5-Dichloro-N-(3,5-dichloro-phenyl)-4-hydroxy-benzenesulfonamide [101];
4-Amino-3-chloro-N-(4-chloro-phenyl)-benzenesulfonamide [102];
3-Chloro-N-(4-chloro-phenyl)-4-methoxy-benzenesulfonamide [103];
N-(3,5-Bis-trifluoromethyl-phenyl)-3-chloro-4-methoxy-benzene-sulfonamide [104];
N-(3-Chloro-4-nitro-phenyl)-3,5-bis-trifluoromethyl-benzenesulfonamide [105];
3-(4-Acetyl-piperazin-1-yl)-N-(3,5-bis-trifluoromethyl-phenyl)-4-nitro-benzenesulfonamide [106];
N-(3,5-Bis-trifluoromethyl-phenyl)-2-nitro-benzenesulfonamide [107];
3-(3,5-Bis-trifluoromethyl-phenylsulfamoyl)-benzoic acid [108];
3,5-Dichloro-N-(4-chloro-benzyl)-4-hydroxy-benzenesulfonamide [109];
3,5-Dichloro-4-hydroxy-N-(4-trifluoromethyl-benzyl)-benzenesulfonamide [110];
3,5-Dichloro-4-hydroxy-N-[2-(1H-indol-3-yl)-ethyl]-benzenesulfonamide [111];
4,5-Dibromo-thiophene-2-sulfonic acid (3,5-dichloro-phenyl)-amide [112];
N-(3,5-Dichloro-phenyl)-4-oxazol-2-yl-benzenesulfonamide [113];
4-Bromo-5-chloro-thiophene-2-sulfonic acid (3,5-bis-trifluoromethyl-phenyl)-amide [114];
4-Bromo-5-chloro-thiophene-2-sulfonic acid (3,5-dichloro-phenyl)-amide [115];
5-Bromo-thiophene-2-sulfonic acid (4-trifluoromethyl-phenyl)-amide [116];
5-Benzenesulfonyl-thiophene-2-sulfonic acid (3,5-bis-trifluoromethyl-phenyl)-amide [117];
5-Benzenesulfonyl-thiophene-2-sulfonic acid (2,4-dichloro-phenyl)-amide [118];
5-Chloro-3-methyl-benzo[b]thiophene-2-sulfonic acid (3,5-bis-trifluoromethyl-phenyl)-amide [119];
Benzo[b]thiophene-2-sulfonic acid (3,5-bis-trifluoromethyl-phenyl)-amide [120];
Benzo[1,2,5]thiadiazole-5-sulfonic acid (4-chloro-phenyl)-amide [121];
Benzo[1,2,5]thiadiazole-5-sulfonic acid (3,5-bis-trifluoromethyl-phenyl)-amide [122];
Benzo[1,2,5]thiadiazole-5-sulfonic acid (4-trifluoromethyl-phenyl)-amide [123];
5-Pyridin-2-yl-thiophene-2-sulfonic acid (3,5-bis-trifluoromethyl-phenyl)-amide [124];
4,5-Dibromo-thiophene-2-sulfonic acid (4-chloro-phenyl)-amide [125];
4,5-Dibromo-thiophene-2-sulfonic acid (4-trifluoromethyl-phenyl)-amide [126];
3,5-Dichloro-N-(4-fluoro-benzyl)-4-hydroxy-benzenesulfonamide [127];
N-(3,5-Bis-trifluoromethyl-phenyl)-2,6-dichloro-benzenesulfonamide [128];
5-Chloro-4-nitro-thiophene-2-sulfonic acid (4-methoxy-2-methyl-phenyl)-amide [129];
5-(3-Hydroxy-piperidin-1-yl)-4-nitro-thiophene-2-sulfonic acid (4-chloro-phenyl)-amide [130]; and
5-Chloro-4-nitro-thiophene-2-sulfonic acid (4-nitro-phenyl)-amide [131].

In a further preferred embodiment, the compound of formula I is selected on the basis that it inhibits human tumour cell proliferation in vitro (using a standard 72-h MTT assay as described in Example 5) with an IC₅₀ value equal to or less than 20 μM.

In a particularly preferred embodiment, the compound of formula I is selected from the following:
5-Chloro-4-nitrothiophene-2-sulfonic acid (4-chlorophenyl)amide [2];
5-Chloro-4-nitrothiophene-2-sulfonic acid (4-fluorophenyl)amide [3];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
4-Chloro-3-nitro-N-(4-trifluoromethylphenyl)-benzene-sulfonamide [35];
4-Chloro-N-(4-chlorophenyl)-3-nitrobenzene-sulfonamide [38];
5-Chloro-4-nitrothiophene-2-sulfonic acid (3,5-dichlorophenyl)amide [39];
5-Chloro-4-nitrothiophene-2-sulfonic acid (3,5-difluorophenyl)amide [40];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethyl-phenyl)-amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
4-Chloro-N-(3,5-dichlorophenyl)-3-nitro-benzenesulfonamide [47];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid (4-chlorophenyl)methylamide [51];
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (4-methoxy-phenyl)-amide [61];
5-Chloro-4-nitro-thiophene-2-sulfonic acid p-tolylamide [63];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
4-Nitro-thiophene-2-sulfonic acid (4-chloro-phenyl)-amide [69];
5-Chloro-4-nitro-thiophene-2-sulfonic acid (3-trifluoro-methyl-benzyl)-(4-trifluoro-methyl-benzyl)-amide [75]; and
N-(1H-Benzoimidazol-2-yl)-4-chloro-3-nitro-benzenesulfonamide [83].

More preferably, the compound of formula I is selected from the following: [2], [3], [5], [6], [7], [35], [39], [40], [44]-[46], [48], [49], [53]-[57], [61], [63], [65]-[67], [75] and [83].

Even more preferably, the compound of formula I is selected from the following: [2], [3], [5], [6], [39], [40], [46], [48], [49], [53], [56], [57], [61], [63], [66], and [83].

More preferably still, more preferably, the compound of formula I is selected from the following: [2], [3], [46], [49], [61], [63] and [83].

In another preferred embodiment, the compound of formula I is selected on the basis that it inhibits p53-HDM2 interaction (using a competitive p53-derived peptide-HDM2 binding assay as described in Example 3) with an IC₅₀ value equal to or less than 200 μM.

Thus, in an especially preferred embodiment of the invention, the compound of formula I is selected from the following:
5-Chloro-4-nitrothiophene-2-sulfonic acid (4-chlorophenyl)amide [2];
5-Chloro-4-nitrothiophene-2-sulfonic acid (4-fluorophenyl)amide [3];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
5-Chlorothiophene-2-sulfonic acid (4-trifluoromethylphenyl)amide [9];
5-Bromothiophene-2-sulfonic acid (3,5-bis-trifluoromethylphenyl)amide [18];
5-Benzenesulfonyl-thiophene-2-sulfonic acid (4-trifluoromethylphenyl)amide [23];
4,5-Dibromothiophene-2-sulfonic acid (3-trifluoromethylphenyl)amide [27];
4,5-Dibromothiophene-2-sulfonic acid (3,4-dichlorophenyl)amide [28];
N-(3,5-Bis-trifluoromethylphenyl)-4-chloro-3-nitrobenzene-sulfonamide [29];
5-Chlorothiophene-2,4-disulfonic acid bis-[(4-trifluoromethylphenyl)amide] [33];
4-Chloro-3-nitro-N-(4-trifluoromethylphenyl)benzene-sulfonamide [35];
5-Chloro-4-nitrothiophene-2-sulfonic acid (3,5-dichlorophenyl)amide [39];
5-Chloro-4-nitrothiophene-2-sulfonic acid (3,5-difluorophenyl)amide [40];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethylphenyl)amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid methyl-(4-trifluoromethylphenyl) amide [50];
5-Chloro-4-nitrothiophene-2-sulfonic acid (4-chlorophenyl)methylamide [51]; and
5-Chloro-4-nitrothiophene-2-sulfonic acid methyl (4-trifluoromethylbenzyl)amide [52]
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (1H-benzo-imidazol-2-yl)-amide [59];
5-Chloro-4-nitro-thiophene-2-sulfonic acid [2-(6-chloro-1H-indol-3-yl)-ethyl]-amide [60];
5-Chloro-4-nitro-thiophene-2-sulfonic acid (4-methoxy-phenyl)-amide [61];
5-Chloro-4-nitro-thiophene-2-sulfonic acid p-tolylamide [63];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
4-Nitro-thiophene-2-sulfonic acid (4-chloro-phenyl)-amide [69];
5-Chloro-thiophene-2,4-disulfonic acid bis-[(4-chloro-phenyl)-amide] [70];
Thioacetic acid S-[5-(4-chloro-phenylsulfamoyl)-3-nitro-thiophen-2-yl]ester [72];
5-Methyl-4-nitro-thiophene-2-sulfonic acid (4-chloro-phenyl)-amide [73];
5-Methyl-thiophene-2,4-disulfonic acid bis-[(4-chloro-phenyl)-amide] [74];
5-Chloro-4-nitro-thiophene-2-sulfonic acid (3-trifluoro-methyl-benzyl)-(4-trifluoromethyl-benzyl)-amide [75];
5-(2-Methoxy-ethylamino)-4-nitro-thiophene-2-sulfonic acid (4-chloro-phenyl)-amide [80];
N-(1H-Benzoimidazol-2-yl)-4-chloro-3-nitro-benzenesulfonamide [83];
5-Chloro-3-methyl-benzo[b]thiophene-2-sulfonic acid (4-chloro-3-nitro-phenyl)-amide [88];
N-(3,5-Bis-trifluoromethyl-phenyl)-3-chloro-4-methoxy-benzene-sulfonamide [104];
N-(3-Chloro-4-nitro-phenyl)-3,5-bis-trifluoromethyl-benzenesulfonamide [105];
4,5-Dibromo-thiophene-2-sulfonic acid (3,5-dichloro-phenyl)-amide [112];
5-Bromo-thiophene-2-sulfonic acid (4-trifluoromethyl-phenyl)-amide [116];
5-Benzenesulfonyl-thiophene-2-sulfonic acid (3,5-bis-trifluoromethyl-phenyl)-amide [117];
5-Chloro-3-methyl-benzo[b]thiophene-2-sulfonic acid (3,5-bis-trifluoromethyl-phenyl)-amide [119];
Benzo[b]thiophene-2-sulfonic acid (3,5-bis-trifluoromethyl-phenyl)-amide [120];
Benzo[1,2,5]thiadiazole-5-sulfonic acid (3,5-bis-trifluoromethyl-phenyl)-amide [122];
4,5-Dibromo-thiophene-2-sulfonic acid (4-chloro-phenyl)-amide [125];
4,5-Dibromo-thiophene-2-sulfonic acid (4-trifluoromethyl-phenyl)-amide [126];
N-(3,5-Bis-trifluoromethyl-phenyl)-2,6-dichloro-benzenesulfonamide [128];
5-(3-Hydroxy-piperidin-1-yl)-4-nitro-thiophene-2-sulfonic acid (4-chloro-phenyl)-amide [130]; and
5-Chloro-4-nitro-thiophene-2-sulfonic acid (4-nitro-phenyl)-amide [131].

More preferably, the compound of formula I is selected from the following: [2], [3], [6], [7], [33], [39], [40], [44]-[46], [48]-[57], [59]-[61], [63], [65]-[67], [72], [75], [130] and [131].

Even more preferably, the compound of formula I is selected from the following: [7], [45], [46], [50]-[53], [65]-[67], [72] and [75].

More preferably still, the compound of formula I is selected from the following: [50], [51], [65], [67] and [75].

Another preferred embodiment of the invention relates to the use of a compound of formula Ic, 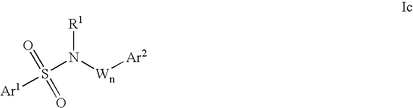
wherein
W is a C₁₋₅ branched or unbranched alkyl group or a C₂₋₅ alkenyl group;
n is 0 or 1;
R¹ is H, a C₁₋₈ branched or unbranched alkyl group, a C₂₋₈ alkenyl group, or an aryl or aralkyl group;
Ar¹ is 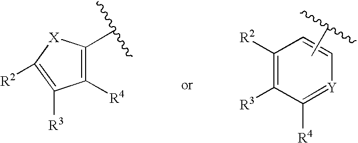
   wherein
   X is S, O, NH or NR′ where R′ is a C₁₋₃ alkyl group;
   Y is CH or N;
   R², R³ and R⁴ are each independently (A)ₚB, wherein A is C₁₋₃ alkyl, p is 0 or 1, and B is H, halogen, C₁₋₅ alkyl, NO₂, OH, NH₂, NHR^{a}, NR^{b}R^{c}, SO₃H, SO₂NH₂, NHAr^{a}, SO₂NHAr^{b}, SO₂NHR^{d}, SO₂Ar^{c}, SO₂R^{e}, CF₃, CN, COOH, COOR^{f}, CONH₂, COONHAr^{d}, CONHR^{g}, COAr^{e}, COR^{h}, or a heteroaryl group optionally substituted by one or more C₁₋₅ alkyl, halogen, SRⁱ or CF₃ groups;
   Ar^{a-e} are each independently aryl groups optionally substituted by one or more C₁₋₅ alkyl, halogen or CF₃ groups;
   R^{a-i} are each independently C₁₋₅ alkyl groups;
   and with the proviso that at least one of R², R³ and R⁴ is other than H;
Ar² is 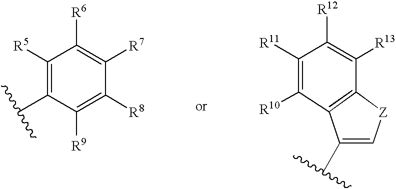
   wherein
   Z is S, O, NH or NR″ where R″ is C₁₋₃ alkyl;
   R⁵, R⁶, R⁷, R⁸ and R⁹ are each independently (L)_{q}M wherein L is C₁₋₃ alkyl, q is 0 or 1, M is H, C₁₋₅ alkyl, halogen, NO₂, OH, NH₂, NHAr, NHR^{j}, NR^{k}R^{l}, SO₃H, SO₂NH₂, SO₂NHAr, SO₂NHR^{m}, SO₂Ar, SO₂Rⁿ, CF₃, CN, COOH, COOR^{p}, CONH₂, CONHAr, CONHR^{q}, COAr or COR^{r};
   R^{i-r} are each independently C₁₋₅ alkyl groups;
   and with the proviso that at least one of the substituents R⁵, R⁶, R⁷, R⁸ and R⁹ is other than H;
R¹⁰, R¹¹, R¹² and R¹³ are each independently H, C₁₋₅ alkyl, halogen, NO₂, OH, NH₂ or CF₃;
   in the preparation of a medicament for treating a proliferative disorder.

In one preferred embodiment, Ar¹ is

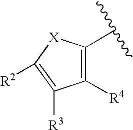

In another preferred embodiment, Ar¹ is

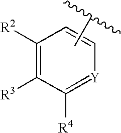

In one preferred embodiment, Ar² is

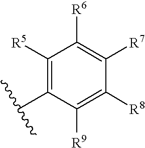

In another preferred embodiment, Ar² is

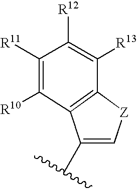

In one preferred embodiment, Ar¹ is 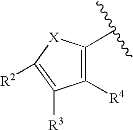
and Ar² is

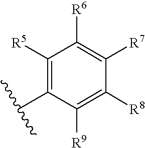

Preferably,
R², R³ and R⁴ are each independently (A)ₚB, wherein A is C₁₋₅ alkyl, p is 0 or 1, and B is H, F, Cl, Br, I, C₁₋₅ alkyl, NO₂, OH, NH₂, NHR^{a}, NR^{b}R^{c}, SO₃H, SO₂NH₂, NHPh, SO₂NHAr, SO₂NHR^{d}, SO₂Ph, SO₂R^{e}, CF₃, CN, COOH, COOR^{f}, CONH₂, COONHPh, CONHR^{g}, COPh, COR^{h}, or a heteroaryl group selected from pyridyl, pyrimidyl, oxazolyl, thiazolyl and pyrazolyl, each of which may be optionally substituted by one or more C₁₋₅ alkyl, halogen, SRⁱ or CF₃ groups; and
R⁵, R⁶, R⁷, R⁸, and R⁹ are each independently (L)_{q}M wherein L is C₁₋₅ alkyl, q is 0 or 1, M is H, C₁₋₅ alkyl, halogen, NO₂, OH, NH₂, NHPh, NHR^{j}, NR^{k}R^{l}, SO₃H, SO₂NH₂, SO₂NHPh, SO₂NHR, SO₂Ph, SO₂R^{m}, CF₃, CN, COOH, COORⁿ, CONH₂, CONHPh, CONHR^{p}, COPh or COR^{q}.

More preferably, R², R³ and R⁴ are each independently (A)ₚB, wherein A is C₁₋₅ alkyl, p is 0 or 1, and B is H, F, Cl, Br, I, C₁₋₅ alkyl, NO₂, OH, NH₂, NHR^{a}, NR^{b}R^{c}, SO₃H, SO₂NH₂, NHPh, SO₂NHPh, SO₂NHR^{d}, SO₂Ph, SO₂R^{e}, CF₃, CN, COOH, COOR^{f}, CONH₂, COONHPh, CONHR^{g}, COPh, COR^{h}, pyridyl, pyrimidyl, 2-methylsulfanylpyrimid-5-yl, oxazol-2-yl, thiazol-2-yl, or 1-methyl-5-trifluoromethyl-1H-pyrazol-4-yl.

More preferably still, R², R³ and R⁴ are each independently H, halogen, NO₂, SO₂Ph, 1-methyl-5-trifluoromethyl-1H-pyrazol-4-yl, 2-methylsulfanyl pyrimid-5-yl, N-(4-fluorophenyl)sulfonamido, N-(4-trifluoromethylphenyl)-sulfonamido, oxazol-2-yl, C₁₋₅ alkyl or NH₂.

In one preferred embodiment, Ar¹ is 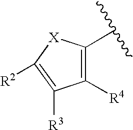
X is S;
R², R³ and R⁴ are each independently H, halogen, NO₂, SO₂Ph, 1-methyl-5-trifluoromethyl-1H-pyrazol-4-yl, 2-methylsulfanylpyrimid-5-yl, N-(4-fluorophenyl)sulfonamido or N-(4-trifluoromethylphenyl)-sulfonamido.

Preferably,
R² is halogen, SO₂Ph, 1-methyl-5-trifluoromethyl-1H-pyrazol-4-yl or 2-methylsulfanyl-pyrimid-5-yl;
R³ is halogen, H, NO₂, N-(4-fluorophenyl)sulfonamido or N-(4-trifluoromethylphenyl)-sulfonamido; and
R⁴ is H.

More preferably, Ar¹ is 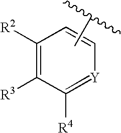
Y is CH or N; and
R², R³ and R⁴ are each independently H, NO₂, NH₂, halogen, oxazol-2-yl or C₁₋₅ alkyl.

More preferably,

when Y is CH

R² is H;
R³ is NO₂ or NH₂; and
R⁴ is H, Cl, oxazol-2-yl or Me; or
   when Y is N
R² is H;
R³ is Br;
R⁴ is Cl.

Preferably,
R⁵, R⁶, R⁷, R⁸ and R⁹ are each independently H, halogen, CF₃ or OH; and
R¹⁰, R¹¹, R¹² and R¹³ are all H.

More preferably,
R⁵ is H or halogen;
R⁶ is H, halogen or CF₃;
R⁷ is H, halogen, OH or CF₃;
R⁹ is H.

Preferably,
W is CH₂, CH₂CH₂ or CH(CH₃)CH₂; and
R¹ is H or Me.
   Theraputic Use

The compounds of the invention have been found to possess anti-proliferative activity and are therefore believed to be of use in the treatment of proliferative disorders, such as cancers, leukaemias or other disorders associated with uncontrolled cellular proliferation such as psoriasis and restenosis.

As defined herein, an anti-proliferative effect within the scope of the present invention may be demonstrated by the ability to inhibit cell proliferation in an in vitro whole cell assay, for example using any of the cell lines AGS, H1299 or SJSA-1, or by showing inhibition of the interaction between HDM2 and p53 in an appropriate assay. These assays, including methods for their performance, are described in more detail in the accompanying Examples. Using such assays it may be determined whether a compound is anti-proliferative in the context of the present invention.

One aspect of the present invention therefore relates to the use of one or more compounds of the invention in the treatment of proliferative disorders.

The term “proliferative disorder” is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the compounds of the present invention may induce apoptosis or maintain stasis within the desired cells as required. Preferably, the proliferative disorder is a cancer or leukaemia.

In one preferred embodiment of the invention, the compound of the invention is administered in an amount sufficient to modulate the interaction between HDM2 and p53.

Even more preferably, the compound of the invention is administered in an amount sufficient to inhibit the interaction between HDM2 and p53.

It is known in the art that HDM2 forms a negative autoregulatory loop with p53 by binding to its N-terminal activation domain thereby inhibiting the functions of p53 and promoting the proteolytic degradation of p53. Interference with this regulatory loop can be used to increase the concentration of active p53 in cells. Thus in tumours with wild-type p53, the equilibrium concentration of active p53 can be increased by antagonising the interaction between HDM2 and p53. This will result in restoration of the p53-mediated pro-apoptotic and anti-proliferative effects in such tumour cells. In tumour types sensitive to increases in functional p53 [Hansen R, Reddel R, Braithwaite A; Oncogene 1995; 11: 2535-2545], it is expected that the compounds of the present invention will be sufficient to induce apoptosis.

The negative regulation of p53 by HDM2 may limit the magnitude of p53 activation by DNA damaging agents currently used (chemotherapy and radiotherapy), thereby limiting their therapeutic effectiveness. Thus if the HDM2 feed-back inhibition of p53 is interrupted, an increase in functional p53 levels will increase the therapeutic effectiveness of such agents by restoring the wild-type p53 function that leads to apoptosis and/or by reversing p53-associated drug resistance. It was thus demonstrated that combining HDM2 inhibition [Wang H, Zeng X, Oliver P, Le L P, Chen J, Chen L, Zhou W, Agrawal S, Zhang R; Int. J. Oncol. 1999; 15: 653-660] and DNA-damaging treatments in vivo led to synergistic anti-tumour effects. Therefore, the compounds of the present invention also have therapeutic applications in sensitising tumour cells for chemotherapy and radiotherapy.

The oncogenic potential of HDM2 is not only determined by its ability to suppress p53, but also by its ability to regulate other tumour suppressor proteins, in particular the retinoblastoma protein pRb and the closely associated E2F1 transcription factor.

Thus, in a further preferred embodiment of the invention, the compound of the invention is administered in an amount sufficient to modulate the interaction between HDM2 and E2F transcription factors.

Even more preferably, the compound of the invention is administered in an amount sufficient to inhibit the interaction between HDM2 and E2F transcription factors.

HDM2 has been shown to interact directly with the pRb-regulated transcription factor E2F1/DP1 [Martin K, Trouche D, Hagemeier C, Sorensen T S, La Thangue N B, Kouzarides T; Nature 1995; 375: 691-694], whose activation capacity it stimulates. Thus overexpression of HDM2 increases E2F1-mediated transactivation [Daujat S, Neel H, Piette J; Trends Genet. 2001; 17: 459-464]. In the absence of p53, HDM2 inhibits the pro-apoptotic effect of E2F1/DP1 by favouring degradation of the heterodimer [Kowalik T F, Degregori J, Leone G, Jakoi L, Nevins J R; Cell Growth Differ. 1998; 9: 113-118]. Simultaneously, however, HDM2 still stimulates DNA synthesis in co-operation with E2F1/DP1. These apparently contradictory observations were reconciled by the proposal that high E2F1/DP1 levels in tumour cells are reduced by HDM2 to appropriate levels for the G1-S phase transition [Loughran O, La Thangue N B; Mol. Cell. Biol. 2000; 20: 2186-2197]. In any case, the anti-apoptotic and growth-promoting activities of HDM2 seem to converge on a single target, E2F1, which could be crucial for the p53-independent oncogenic activities of HDM2 [Daujat S et al, ibid].

A domain of E2F1 (amino acids 390 to 406) shows striking similarity to the HDM2-binding domain of p53. Since the interactions of HDM2 with both p53 and E2F1 locate to the same binding site on HDM2, it can be expected that HDM2/p53 antagonists, such as the compounds of the present invention, will not only activate cellular p53 but also suppress E2F1 activities, which are commonly deregulated in tumour cells. Furthermore, it can be expected that the compounds of the invention will exhibit anti-proliferative effects in tumour cells, even if such cells are devoid of functional p53.

Compounds

Another aspect of the invention relates to compounds of formula Ia, 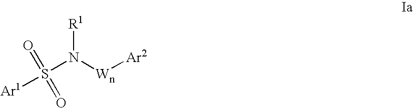
wherein
W is a C₁₋₅ branched or unbranched alkylene group or a C₂₋₅ alkenylene group;
n is 0 or 1;
R¹ is H, a C₁₋₈ branched or unbranched alkyl group, a C₂₋₈ alkenyl group, or an aryl or aralkyl group, each of which may be optionally substituted by one or more halogen or CF₃ groups;
Ar¹ is 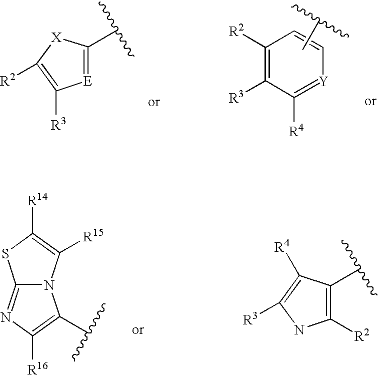
   wherein
   X is O, NH or NR′ where R′ is a C₁₋₃ alkyl group;
   E is N or CR⁴;
   Y is N;
   R², R³, R⁴, and R¹⁴⁻¹⁶ are each independently (A)ₚB, wherein A is C₁₋₃ alkyl, p is 0 or 1, and B is H, halogen, C₁₋₅ alkyl, NO₂, OH, NH₂, NHR^{a}, NR^{b}R^{c}, SO₃H, SO₂NH₂, NHAr^{a}, SO₂NHAr^{b}, SO₂NHR^{d}, SO₂Ar^{c}, SO₂R^{e}, CF₃, CN, COOH, COOR^{f}, CONH₂, COONHAr^{d}, CONHR^{g}, COAr^{e}, COR^{h}, S(CO)R^{s}, OR^{t}, OAr^{f}, an alicyclic group optionally containing one or more heteroatoms, optionally substituted by one or more OH, COR^{u}, halogen or CF₃ groups, or a heteroaryl group optionally substituted by one or more C₁₋₅ alkyl, halogen, SRⁱ or CF₃ groups; or
   R² and R³ are linked to form a saturated or unsaturated ring system, optionally containing one or more heteroatoms, and optionally substituted by one or more halogen, OH or CF₃ groups;
   Ar^{a-f} are each independently aryl groups optionally substituted by one or more C₁₋₅ alkyl, halogen or CF₃ groups;
   R^{a-i}, R^{s}, R^{t} and R^{u} are each independently C₁₋₅ alkyl groups optionally substituted by one or more alkoxy, halogen or CF3 groups;
   and with the proviso that at least one of R², R³ and R⁴ is other than H;
Ar² is 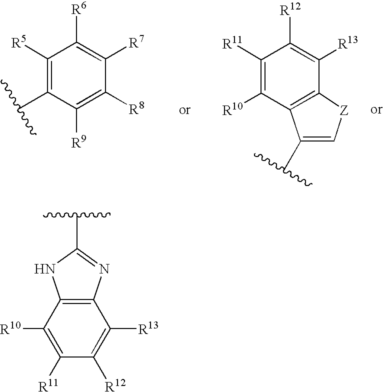
   wherein
   Z is S, O, NH or NR″ where R″ is C₁₋₃ alkyl;
   R⁵, R⁶, R⁷, R⁸ and R⁹ are each independently (L)_{q}M wherein L is C₁₋₃ alkyl, q is 0 or 1, M is H, C₁₋₅ alkyl, halogen, NO₂, OH, NH₂, NHAr^{g}, NHR^{j}, NR^{k}R^{l}, SO₃H, SO₂NH₂, SO₂NHAr^{h}, SO₂NHR^{m}, SO₂Arⁱ, SO₂Rⁿ, CF₃, CN, COOH, COOR^{p}, CONH₂, CONHAr^{j}, CONHR^{q}, OR^{v}, COAr^{k} or COR^{r};
   R^{j-r}, R^{v} are each independently C₁₋₅ alkyl groups;
   Ar^{g-k} are each independently aryl groups;
   and with the proviso that at least one of the substituents R⁵, R⁶, R⁷, R⁸ and R⁹ is other than H;
   R¹⁰, R¹¹, R¹² and R¹³ are each independently H, C₁₋₅ alkyl, halogen, NO₂, OH, NH₂ or CF₃;
      with the proviso that said compound is other than 5-[(4-chlorophenyl)amino]sulfonyl-2-furancarboxylic acid.

Another aspect of the invention relates to compounds of formula Ib, 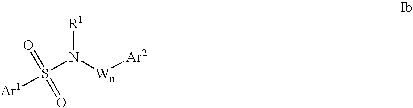
wherein
W is a C₁₋₅ branched or unbranched alkylene group or a C₂₋₅ alkenylene group;
n is 0 or 1;
R¹ is H, a C₁₋₈ branched or unbranched alkyl group, a C₂₋₈ alkenyl group, or an aryl or aralkyl group, each of which may be optionally substituted by one or more halogen or CF₃ groups;
Ar¹ is 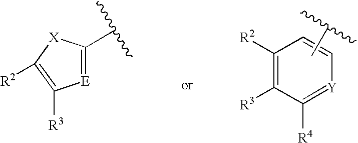
   wherein
   X is S, O, NH or NR′ where R′ is a C₁₋₃ alkyl group;
   E is N or CR⁴;
   Y is CH or N;
   R², R³ and R⁴ are each independently (A)ₚB, wherein A is C₁₋₃ alkyl, p is 0 or 1, and B is H, halogen, C₁₋₅ alkyl, NO₂, OH, NH₂, NHR^{a}, NR^{b}R^{c}, SO₃H, SO₂NH₂, NHAr^{a}, SO₂NHAr^{b}, SO₂NHR^{d}, SO₂Ar^{c}, SO₂R^{e}, CF₃, CN, COOH, COOR^{f}, CONH₂, COONHAr^{d}, CONHR^{g}, COAr^{e}, COR^{h}, S(CO)R^{s}, OR^{t}, OAr^{f}, an alicyclic group optionally containing one or more heteroatoms, optionally substituted by one or more OH, COR^{u}, halogen or CF₃ groups, or a heteroaryl group optionally substituted by one or more C₁₋₅ alkyl, halogen, SRⁱ or CF₃ groups; or
   R² and R³ are linked to form a saturated or unsaturated ring system, optionally containing one or more heteroatoms, and optionally substituted by one or more halogen, OH or CF₃ groups;
   Ar^{a-f} are each independently aryl groups optionally substituted by one or more C₁₋₅ alkyl, halogen or CF₃ groups;
   R^{a-i}, R^{s}, R^{t} and R^{u} are each independently C₁₋₅ alkyl groups optionally substituted by one or more alkoxy, halogen or CF₃ groups;
   and with the proviso that at least one of R², R³ and R⁴ is other than H;
Ar² is 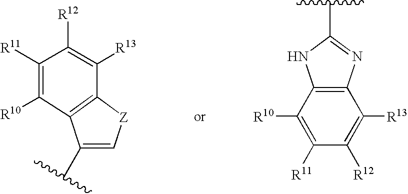
   wherein
   Z is S, O, NH or NR″ where R″ is C₁₋₃ alkyl;
   R⁵, R⁶, R⁷, R⁸ and R⁹ are each independently (L)_{q}M wherein L is C₁₋₃ alkyl, q is 0 or 1, M is H, C₁₋₅ alkyl, halogen, NO₂, OH, NH₂, NHAr^{g}, NHR^{j}, NR^{k}R^{l}, SO₃H, SO₂NH₂, SO₂NHAr^{h}, SO₂NHR^{m}, SO₂Arⁱ, SO₂Rⁿ, CF₃, CN, COOH, COOR^{p}, CONH₂, CONHAr^{j}, CONHR^{q}, OR^{v}, COAr^{k} or COR^{r};
   R^{j-r}, R^{v} are each independently C₁₋₅ alkyl groups;
   Ar^{g-k} are each independently aryl groups;
   and with the proviso that at least one of the substituents R⁵, R⁶, R⁷, R⁸ and R⁹ is other than H;
   R¹⁰, R¹¹, R¹² and R¹³ are each independently H, C₁₋₅ alkyl, halogen, NO₂, OH, NH₂ or CF₃.

Another aspect of the invention relates to compounds of formula Id, 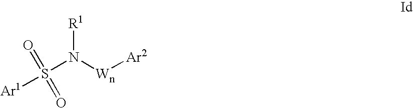
wherein
W is a C₁₋₅ branched or unbranched alkyl group or a C₂₋₅ alkenyl group;
n is 0 or 1;
R¹ is H, a C₁₋₈ branched or unbranched alkyl group, a C₂₋₈ alkenyl group, or an aryl or aralkyl group;
Ar¹ is 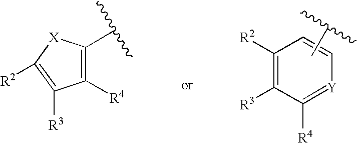
   wherein
   X is O, NH or NR′ where R′ is a C₁₋₃ alkyl group;
   Y is CH or N;
   R², R³ and R⁴ are each independently (A)ₚB, wherein A is C₁₋₃ alkyl, p is 0 or 1, and B is H, halogen, C₁₋₅ alkyl, NO₂, OH, NH₂, NHR^{a}, NR^{b}R^{c}, SO₃H, SO₂NH₂, NHAr^{a}, SO₂NHAr^{b}, SO₂NHR^{d}, SO₂Ar^{c}, SO₂R^{e}, CF₃, CN, COOH, COOR^{f} CONH₂, COONHAr^{d}, CONHR^{g}, COAr^{e}, COR^{h}, or a heteroaryl group optionally substituted by one or more C₁₋₅ alkyl, halogen, SRⁱ or CF₃ groups;
   Ar^{a-e} are each independently aryl groups optionally substituted by one or more C₁₋₅ alkyl, halogen or CF₃ groups;
   R^{a-i} are each independently C₁₋₅ alkyl groups;
   and with the proviso that at least one of R², R³ and R⁴ is other than H;
Ar² is 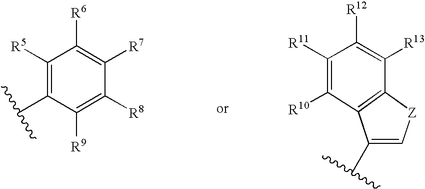
   wherein
   Z is S, O, NH or NR″ where R″ is C₁₋₃ alkyl;
   R⁵, R⁶, R⁷, R⁸ and R⁹ are each independently (L)_{q}M wherein L is C₁₋₃ alkyl, q is 0 or 1, M is H, C₁₋₅ alkyl, halogen, NO₂, OH, NH₂, NHAr, NHR^{j}, NR^{k}R^{l}, SO₃H, SO₂NH₂, SO₂NHAr, SO₂NHR^{m}, SO₂Ar, SO₂Rⁿ, CF₃, CN, COOH, COOR^{p}, CONH₂, CONHAr, CONHR^{q}, COAr or COR^{r};
   R^{i-r} are each independently C₁₋₅ alkyl groups;
   and with the proviso that at least one of the substituents R⁵, R⁶, R⁷, R⁸ and R⁹ is other than H;
   R¹⁰, R¹¹, R¹² and R¹³ are each independently H, C₁₋₅ alkyl, halogen, NO₂, OH, NH₂ or CF₃;
      with the proviso that said compound is other than
5-[(4-chlorophenyl)amino]sulfonyl-2-furancarboxylic acid;
N-[3,5-bis(trifluoromethyl)phenyl]-4-chloro-3-nitrobenzensulfonamide;
2,6-dichloro-N-[4-chloro-3-(3-diethylarinopropyl)-phenyl]4-trifluoromethylbenzenesulfonamide; or
2,6-dichloro-N-[4-chloro-3-(3-dimethylaminopropyl)-phenyl]4-trifluoromethylbenzenesulfonamide.

Preferred embodiments in respect of compounds of formula 1a, 1b and 1d are as defined above for compounds of formula I.

Another aspect of the invention relates to a compound selected from the following:
5-Chloro-4-nitrothiophene-2-sulfonic acid (3-trifluoromethylphenyl)amide [1];
5-Chloro-4-nitrothiophene-2-sulfonic acid (4-chlorophenyl)-amide [2];
5-Chloro-4-nitrothiophene-2-sulfonic acid (4-fluorophenyl)amide [3];
4-Bromo-5-chlorothiophene-2-sulfonic acid (4-fluorophenyl)amide [4];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
4,5-Dibromothiophene-2-sulfonic acid (3,5-bis-trifluoromethylphenyl)amide [8];
5-Chlorothiophene-2-sulfonic acid (3,5-bis-trifluoromethylphenyl)amide [11];
5-(2-Methylsulfanyl-pyrimidin-5-yl)-thiophene-2-sulfonic acid (3,5-dichlorophenyl)-amide [12];
4-Oxazol-2-yl-N-(4-trifluoromethylphenyl)benzenesulfonamide [13];
N-(3,5-Bis-trifluoromethylphenyl)-4-oxazol-2-yl-benzenesulfonamide [14];
4-Bromo-5-chlorothiophene-2-sulfonic acid (4-trifluoromethylphenyl)amide [15];
5-Bromothiophene-2-sulfonic acid (4-chlorophenyl)amide [16];
5-Bromothiophene-2-sulfonic acid (3,5-dichlorophenyl)amide [17];
5-Bromothiophene-2-sulfonic acid (3,5-bis-trifluoromethylphenyl)amide [18];
N-(4-Chlorophenyl)-3-nitrobenzenesulfonamide [19];
3-Nitro-N-(4-trifluoromethylphenyl)benzenesulfonamide [20];
N-(3,5-Bis-trifluoromethylphenyl)-3-nitrobenzenesulfonamide [21];
N-(2,4-Dichlorophenyl)-3-nitrobenzenesulfonamide [22];
5-Benzenesulfonylthiophene-2-sulfonic acid (4-trifluoromethylphenyl)-amide [23];
5-Benzenesulfonylthiophene-2-sulfonic acid (4-chlorophenyl)amide [24];
5-Benzenesulfonylthiophene-2-sulfonic acid (3,5-dichlorophenyl)amide [25];
5-Chlorothiophene-2-sulfonic acid (3,4-dichlorophenyl)amide [26];
4,5-Dibromothiophene-2-sulfonic acid (3-trifluoromethylphenyl)amide [27];
4,5-Dibromothiophene-2-sulfonic acid (3,4-dichlorophenyl)amide [28];
4-Chloro-N-(3,4-dichlorophenyl)-3-nitrobenzenesulfonamide [30];
5-(1-Methyl-5-trifluoromethyl-1H-pyrazol-4-yl)-thiophene-2-sulfonic acid (4-trifluoromethylphenyl)-amide [31];
5-Chlorothiophene-2,4-disulfonic acid bis-[(4-fluorophenyl)-amide] [32];
5-Chlorothiophene-2,4-disulfonic acid bis-[(4-trifluoro-methyl-phenyl)-amide] [33];
4-Methyl-3-nitro-N-(4-trifluoromethylphenyl)benzenesulfonamide [34];
4-Chloro-3-nitro-N-(4-trifluoromethylphenyl)benzenesulfonamide [35];
3-Amino-4-methyl-N-(4-trifluoromethyl-phenyl)benzenesulfonamide [36];
N-(4-Chlorophenyl)-4-methyl-3-nitrobenzenesulfonamide [37];
4-Chloro-N-(4-chlorophenyl)-3-nitro-benzenesulfonamide [38];
5-Chloro-4-nitrothiophene-2-sulfonic acid (3,5-dichlorophenyl)-amide [39];
5-Chloro-4-nitrothiophene-2-sulfonic acid (3,5-difluorophenyl)-amide [40];
5-Bromo-6-chloropyridine-3-sulfonic acid (4-trifluoromethylphenyl)amide [41];
5-Bromo-6-chloropyridine-3-sulfonic acid (3,5-bis-trifluoromethylphenyl)amide [42];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethyl-phenyl)-amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
4-Chloro-N-(3,5-dichlorophenyl)-3-nitro-benzenesulfonamide [47];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid methyl-(4-trifluoromethylphenyl)amide [50];
5-Chloro-4-nitrothiophene-2-sulfonic acid (4-chloro-phenyl)-methylamide [51]; and
5-Chloro-4-nitrothiophene-2-sulfonic acid methyl-(4-trifluoromethylbenzyl)amide [52];
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (4-chloro-phenyl)-isobutyl-amide [58];
5-Chloro-4-nitro-thiophene-2-sulfonic acid (1H-benzo-imidazol-2-yl)-amide [59];
5-Chloro-4-nitro-thiophene-2-sulfonic acid [2-(6-chloro-1H-indol-3-yl)-ethyl]-amide [60];
5-Chloro-4-nitro-thiophene-2-sulfonic acid (4-methoxy-phenyl)-amide [61];
5-Chloro-4-nitro-thiophene-2-sulfonic acid phenylamide [62];
5-Chloro-4-nitro-thiophene-2-sulfonic acid p-tolylamide [63];
5-Chloro-4-nitro-thiophene-2-sulfonic acid benzylamide [64];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
5-Nitro-thiophene-2-sulfonic acid (4-chloro-phenyl)-amide [68];
4-Nitro-thiophene-2-sulfonic acid (4-chloro-phenyl)-amide [69];
5-Chloro-thiophene-2,4-disulfonic acid bis-[(4-chloro-phenyl)-amide] [70];
5-Ethyl-4-nitro-thiophene-2-sulfonic acid (4-chloro-phenyl)-amide [71];
Thioacetic acid S-[5-(4-chloro-phenylsulfamoyl)-3-nitro-thiophen-2-yl]ester [72];
5-Methyl-4-nitro-thiophene-2-sulfonic acid (4-chloro-phenyl)-amide [73];
5-Methyl-thiophene-2,4-disulfonic acid bis-[(4-chloro-phenyl)-amide] [74];
5-Chloro-4-nitro-thiophene-2-sulfonic acid (3-trifluoro-methyl-benzyl)-(4-trifluoromethyl-benzyl)-amide [75];
4-Nitro-thiophene-2-sulfonic acid (4-trifluoromethyl-phenyl)-amide [76];
4-Nitro-thiophene-2-sulfonic acid [2-(1H-indol-3-yl)-ethyl]-amide [77];
5-(1-Methyl-5-tri-fluoromethyl-1H-pyrazol-3-yl)-thiophene-2-sulfonic acid (3,5-bis-trifluoromethyl-phenyl)-(3-trifluoro-methyl-benzyl)-amide [78];
5-Morpholin-4-yl-4-nitro-thiophene-2-sulfonic acid (4-chloro-phenyl)-amide [79];
5-(2-Methoxy-ethylamino)-4-nitro-thiophene-2-sulfonic acid (4-chloro-phenyl)-amide [80];
4-Chloro-N-[2-(5-chloro-1H-indol-3-yl)-ethyl]-3-nitro-benzenesulfonamide [81];
N-[2-(5-Chloro-1H-indol-3-yl)-ethyl]-4-methyl-3-nitro-benzenesulfonamide [82];
N-(1H-Benzoimidazol-2-yl)-4-chloro-3-nitro-benzenesulfonamide [83];
6-Chloro-imidazo[2,1-b]thiazole-5-sulfonic acid (3,5-bis-trifluoro-methyl-phenyl)-amide [84];
2,3-Dihydro-benzo[1,4]dioxine-6-sulfonic acid (4-chloro-phenyl)-amide [85];
2,3-Dihydro-benzo[1,4]dioxine-6-sulfonic acid (3,5-bis-trifluoromethyl-phenyl)-amide [86];
6-Phenoxy-pyridine-3-sulfonic acid (4-chloro-phenyl)-amide [87];
5-Chloro-3-methyl-benzo[b]thiophene-2-sulfonic acid (4-chloro-3-nitro-phenyl)-amide [88];
N-(3,5-Bis-trifluoromethyl-phenyl)-4-pyrazol-1-yl-benzenesulfonamide [89];
4-(4-Chloro-phenylsulfamoyl)-3,5-dimethyl-1H-pyrrole-2-carboxylic acid ethyl ester [90];
4-(3,5-Bis-trifluoromethyl-phenylsulfamoyl)-3,5-dimethyl-1H-pyrrole-2-carboxylic acid [91];
4-(4-Chloro-phenylsulfamoyl)-3,5-dimethyl-1H-pyrrole-2-carboxylic acid [92];
2-(4-Chloro-phenylsulfamoyl)-4-methyl-thiazole-5-carboxylic acid ethyl ester [93];
3,5-Dichloro-N-(4-chloro-phenyl)-4-hydroxy-benzenesulfonamide [94];
N-(3,5-Bis-trifluoromethyl-phenyl)-3,5-dichloro-4-hydroxy-benzenesulfonamide [95];
3,5-Dichloro-4-hydroxy-N-(4-trifluoromethyl-phenyl)-benzenesulfonamide [96];
N-(4-Chloro-phenyl)-4-nitro-benzene-sulfonamide [97];
N-(3,5-Bis-trifluoromethyl-phenyl)-4-nitro-benzenesulfonamide [98];
4-Amino-N-(3,5-bis-trifluoromethyl-phenyl)-3-chloro-benzenesulfonamide [99];
3-Nitro-N-(4-trifluoromethyl-phenyl)-benzenesulfonamide [100];
3,5-Dichloro-N-(3,5-dichloro-phenyl)-4-hydroxy-benzenesulfonamide [101];
4-Amino-3-chloro-N-(4-chloro-phenyl)-benzenesulfonamide [102];
3-Chloro-N-(4-chloro-phenyl)-4-methoxy-benzenesulfonamide [103];
N-(3,5-Bis-trifluoromethyl-phenyl)-3-chloro-4-methoxy-benzene-sulfonamide [104];
N-(3-Chloro-4-nitro-phenyl)-3,5-bis-trifluoromethyl-benzenesulfonamide [105];
3-(4-Acetyl-piperazin-1-yl)-N-(3,5-bis-trifluoromethyl-phenyl)-4-nitro-benzenesulfonamide [106];
N-(3,5-Bis-trifluoromethyl-phenyl)-2-nitro-benzenesulfonamide [107];
3-(3,5-Bis-trifluoromethyl-phenylsulfamoyl)-benzoic acid [108];
3,5-Dichloro-N-(4-chloro-benzyl)-4-hydroxy-benzenesulfonamide [109];
3,5-Dichloro-4-hydroxy-N-(4-trifluoromethyl-benzyl)-benzenesulfonamide [110];
3,5-Dichloro-4-hydroxy-N-[2-(1H-indol-3-yl)-ethyl]-benzenesulfonamide [111];
4,5-Dibromo-thiophene-2-sulfonic acid (3,5-dichloro-phenyl)-amide [112];
N-(3,5-Dichloro-phenyl)-4-oxazol-2-yl-benzenesulfonamide [113];
4-Bromo-5-chloro-thiophene-2-sulfonic acid (3,5-bis-trifluoromethyl-phenyl)-amide [114];
4-Bromo-5-chloro-thiophene-2-sulfonic acid (3,5-dichloro-phenyl)-amide [115];
5-Benzenesulfonyl-thiophene-2-sulfonic acid (3,5-bis-trifluoromethyl-phenyl)-amide [117];
5-Benzenesulfonyl-thiophene-2-sulfonic acid (2,4-dichloro-phenyl)-amide [118];
5-Chloro-3-methyl-benzo[b]thiophene-2-sulfonic acid (3,5-bis-trifluoromethyl-phenyl)-amide [119];
Benzo[b]thiophene-2-sulfonic acid (3,5-bis-trifluoromethyl-phenyl)-amide [120];
Benzo[1,2,5]thiadiazole-5-sulfonic acid (4-chloro-phenyl)-amide [121];
Benzo[1,2,5]thiadiazole-5-sulfonic acid (3,5-bis-trifluoromethyl-phenyl)-amide [122];
Benzo[1,2,5]thiadiazole-5-sulfonic acid (4-trifluoromethyl-phenyl)-amide [123];
5-Pyridin-2-yl-thiophene-2-sulfonic acid (3,5-bis-trifluoromethyl-phenyl)-amide [124];
4,5-Dibromo-thiophene-2-sulfonic acid (4-chloro-phenyl)-amide [125];
4,5-Dibromo-thiophene-2-sulfonic acid (4-trifluoromethyl-phenyl)-amide [126];
3,5-Dichloro-N-(4-fluoro-benzyl)-4-hydroxy-benzenesulfonamide [127];
N-(3,5-Bis-trifluoromethyl-phenyl)-2,6-dichloro-benzenesulfonamide [128];
5-Chloro-4-nitro-thiophene-2-sulfonic acid (4-methoxy-2-methyl-phenyl)-amide [129];
5-(3-Hydroxy-piperidin-1-yl)-4-nitro-thiophene-2-sulfonic acid (4-chloro-phenyl)-amide [130]; and
5-Chloro-4-nitro-thiophene-2-sulfonic acid (4-nitro-phenyl)-amide [131].

Preferably, the compound is selected from the following:
5-Chloro-4-nitrothiophene-2-sulfonic acid (4-chlorophenyl)amide [2];
5-Chloro-4-nitrothiophene-2-sulfonic acid (4-fluorophenyl)amide [3];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
4-Chloro-3-nitro-N-(4-trifluoromethylphenyl)-benzene-sulfonamide [35];
4-Chloro-N-(4-chlorophenyl)-3-nitrobenzene-sulfonamide [38];
5-Chloro-4-nitrothiophene-2-sulfonic acid (3,5-dichlorophenyl)amide [39];
5-Chloro-4-nitrothiophene-2-sulfonic acid (3,5-difluorophenyl)amide [40];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethyl-phenyl)-amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid (4-chlorophenyl)methylamide [51];
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (4-methoxy-phenyl)-amide [61];
5-Chloro-4-nitro-thiophene-2-sulfonic acid p-tolylamide [63];
5-Chloro-4-nitro-thiophene-2-sulfonic acid benzylamide [64];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
4-Nitro-thiophene-2-sulfonic acid (4-chloro-phenyl)-amide [69];
5-Chloro-4-nitro-thiophene-2-sulfonic acid (3-trifluoro-methyl-benzyl)-(4-trifluoromethyl-benzyl)-amide [75]; and
N-(1H-Benzoimidazol-2-yl)-4-chloro-3-nitro-benzenesulfonamide [83].

More preferably, the compound of formula I is selected from the following: [2], [3], [5], [6], [7], [35], [39], [40], [44]-[46], [48], [49], [53]-[57], [61], [63]-[67], [75] and [83].

Even more preferably, the compound of formula I is selected from the following: [2], [3], [5], [6], [39], [40], [46], [48], [49], [53], [56], [57], [61], [63], [64], [66] and [83].

More preferably still, the compound of formula I is selected from the following: [2], [3], [46], [49], [61], [63] and [83].

In another preferred embodiment, the compound is selected from the following:
5-Chloro-4-nitrothiophene-2-sulfonic acid (4-chlorophenyl)amide [2];
5-Chloro-4-nitrothiophene-2-sulfonic acid (4-fluorophenyl)amide [3];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
5-Bromothiophene-2-sulfonic acid (3,5-bis-trifluoromethylphenyl)amide [18];
5-Benzenesulfonyl-thiophene-2-sulfonic acid (4-trifluoromethylphenyl)amide [23];
4,5-Dibromothiophene-2-sulfonic acid (3-trifluoromethylphenyl)amide [27];
4,5-Dibromothiophene-2-sulfonic acid (3,4-dichlorophenyl)amide [28];
5-Chlorothiophene-2,4-disulfonic acid bis-[(4-trifluoromethylphenyl)amide] [33];
4-Chloro-3-nitro-N-(4-trifluoromethylphenyl)benzene-sulfonamide [35];
5-Chloro-4-nitrothiophene-2-sulfonic acid (3,5-dichlorophenyl)amide [39];
5-Chloro-4-nitrothiophene-2-sulfonic acid (3,5-difluorophenyl)amide [40];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethylphenyl)amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid methyl-(4-trifluoromethylphenyl) amide [50];
5-Chloro-4-nitrothiophene-2-sulfonic acid (4-chlorophenyl)methylamide [51]; and
5-Chloro-4-nitrothiophene-2-sulfonic acid methyl (4-trifluoromethylbenzyl)amide [52]
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (1H-benzo-imidazol-2-yl)-amide [59];
5-Chloro-4-nitro-thiophene-2-sulfonic acid [2-(6-chloro-1H-indol-3-yl)-ethyl]-amide [60];
5-Chloro-4-nitro-thiophene-2-sulfonic acid (4-methoxy-phenyl)-amide [61];
5-Chloro-4-nitro-thiophene-2-sulfonic acid phenylamide [62];
5-Chloro-4-nitro-thiophene-2-sulfonic acid p-tolylamide [63];
5-Chloro-4-nitro-thiophene-2-sulfonic acid benzylamide [64];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
4-Nitro-thiophene-2-sulfonic acid (4-chloro-phenyl)-amide [69];
5-Chloro-thiophene-2,4-disulfonic acid bis-[(4-chloro-phenyl)-amide] [70];
Thioacetic acid S-[5-(4-chloro-phenylsulfamoyl)-3-nitro-thiophen-2-yl]ester [72];
5-Methyl-4-nitro-thiophene-2-sulfonic acid (4-chloro-phenyl)-amide [73];
5-Methyl-thiophene-2,4-disulfonic acid bis-[(4-chloro-phenyl)-amide] [74];
5-Chloro-4-nitro-thiophene-2-sulfonic acid (3-trifluoro-methyl-benzyl)-(4-trifluoromethyl-benzyl)-amide [75];
5-(2-Methoxy-ethylamino)-4-nitro-thiophene-2-sulfonic acid (4-chloro-phenyl)-amide [80];
N-(1H-Benzoimidazol-2-yl)-4-chloro-3-nitro-benzenesulfonamide [83];
5-Chloro-3-methyl-benzo[b]thiophene-2-sulfonic acid (4-chloro-3-nitro-phenyl)-amide [88];
N-(3,5-Bis-trifluoromethyl-phenyl)-3-chloro-4-methoxy-benzene-sulfonamide [104];
N-(3-Chloro-4-nitro-phenyl)-3,5-bis-trifluoromethyl-benzenesulfonamide [105];
4,5-Dibromo-thiophene-2-sulfonic acid (3,5-dichloro-phenyl)-amide [112];
5-Benzenesulfonyl-thiophene-2-sulfonic acid (3,5-bis-trifluoromethyl-phenyl)-amide [117];
5-Chloro-3-methyl-benzo[b]thiophene-2-sulfonic acid (3,5-bis-trifluoromethyl-phenyl)-amide [119];
Benzo[b]thiophene-2-sulfonic acid (3,5-bis-trifluoromethyl-phenyl)-amide [120];
Benzo[1,2,5]thiadiazole-5-sulfonic acid (3,5-bis-trifluoromethyl-phenyl)-amide [122];
4,5-Dibromo-thiophene-2-sulfonic acid (4-chloro-phenyl)-amide [125];
4,5-Dibromo-thiophene-2-sulfonic acid (4-trifluoromethyl-phenyl)-amide [126];
N-(3,5-Bis-trifluoromethyl-phenyl)-2,6-dichloro-benzenesulfonamide [128];
5-(3-Hydroxy-piperidin-1-yl)-4-nitro-thiophene-2-sulfonic acid (4-chloro-phenyl)-amide [130]; and
5-Chloro-4-nitro-thiophene-2-sulfonic acid (4-nitro-phenyl)-amide [131].

More preferably, the compound is selected from the following: [2], [3], [6], [7], [33], [39], [40], [44]-[46], [48]-[57], [59]-[67], [72], [75], [130] and [131].

Even more preferably, the compound is selected from the following: [7], [45], [46], [50]-[53], [64]-[67], [72] and [75].

More preferably still, the compound is selected from the following: [50], [51], [65], [67] and [75].

The invention also relates to the use of the above-mentioned compounds in the preparation of a medicament for treating a proliferative disorder.

Pharmacuetical Composistions

A fourth aspect of the invention relates to a pharmaceutical composition comprising a compound of formula I as defined for said first and third aspects admixed with one or more pharmaceutically acceptable diluents, excipients or carriers. Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The compounds of the present invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects of the present invention previously discussed, the invention includes, where appropriate all enantiomers and tautomers of compounds of the invention. The man skilled in the art will recognise compounds that possess an optical properties (one or more chiral carbon atoms) or tautomeric characteristics. The corresponding enantiomers and/or tautomers may be isolated/prepared by methods known in the art.

Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes the use of solvate forms of the compounds of the present invention. The terms used in the claims encompass these forms.

Polymorphs

The invention furthermore relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes the compounds of the invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient for the treatment of malignancy.

Combinations

A further aspect of the present invention relates to a combination comprising at least one compound of the invention as defined above and at least one cytotoxic agent.

Preferably, the combination is a synergistic combination. Thus, preferably, the compound of the invention is capable of synergistically interacting with the one or more other cytotoxic agents, for example, to enhance the cytotoxic effect of the other agent.

In one preferred embodiment, the cytotoxic agent is a chemotherapeutic agent.

In a particularly preferred embodiment, the chemotherapeutic agent is cisplatin or etoposide. Even more preferably, the compound of the invention is capable of exhibiting a chemosensitisation effect, for example, by interacting synergistically to increase the cytotoxic effects of cisplatin and etoposide. In other words, the combined action of the compound of the invention and the cytotoxic agent produces a greater effect than would be expected from adding the individual effects of each component. Further details regarding the synergistic effect may be found in the accompanying examples.

Another aspect of the invention relates to a pharmaceutical composition comprising at least one compound of the invention as defined above, and one or more cytotoxic agents, admixed with a pharmaceutically acceptable diluent, excipient or carrier.

In a particularly preferred embodiment, the one or more compounds of the invention are administered in combination with one or more other anticancer agents, for example, existing anticancer drugs available on the market. In such cases, the compounds of the invention may be administered consecutively, simultaneously or sequentially with the one or more other anticancer agents.

Thus, one aspect of the invention provides a method of treating a proliferative disorder, said method comprising administering to a subject at least one compound of the invention as defined above consecutively, simultaneously or sequentially with one or more other cytotoxic agents.

Anticancer drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in early tumor cells which would have been otherwise responsive to initial chemotherapy with a single agent. An example of the use of biochemical interactions in selecting drug combinations is demonstrated by the administration of leucovorin to increase the binding of an active intracellular metabolite of 5-fluorouracil to its target, thymidylate synthase, thus increasing its cytotoxic effects.

Numerous combinations are used in current treatments of cancer and leukemia. A more extensive review of medical practices may be found in “Oncologic Therapies” edited by E. E. Vokes and H. M. Golomb, published by Springer.

Beneficial combinations may be suggested by studying the activity of the test compounds with agents known or suspected of being valuable in the treatment of a particular cancer initially or cell lines derived from that cancer. This procedure can also be used to determine the order of administration of the agents, i.e. before, simultaneously, or after delivery. Such scheduling may be a feature of all the cycle acting agents identified herein.

The compounds of the present invention may also be used in combination with radiotherapy treatment. Thus, another aspect of the invention provides a method of treating a proliferative disorder, said method comprising administering to a subject at least one compound of formula I as defined above consecutively, simultaneously or sequentially with radiotherapy.

Assays

A fifth aspect of the invention relates to the use of a compound of formula I, as defined in the above-mentioned first aspect, in an assay for determining binding to HDM2.

Preferably, the assay is capable of identifying candidate compounds that influence the activity of HDM2 on p53 and/or E2F.

More preferably still, the assay is capable of identifying candidate compounds that inhibit the interaction between HDM2 and p53 and/or E2F.

Even more preferably, the assay is a competitive binding assay.

Preferably, the competitive binding assay comprises contacting a compound of formula I as defined in the above-mentioned first aspect of the invention with HDM2 in the presence of a p53-derived peptide and detecting any change in the interaction between HDM2 and said p53-derived peptide.

In a particularly preferred embodiment, said p53-derived peptide is a fluorescently labelled or biotinylated p53-derived peptide.

A sixth aspect of the invention provides a method of detecting the binding of a ligand to HDM2, said method comprising the steps of:

(i) contacting a ligand with HDM2 in the presence of a p53-derived peptide; and

(ii) detecting any change in the interaction between HDM2 and said p53-derived peptide;

and wherein said ligand is a compound according to the above-mentioned first aspect of the invention.

One aspect of the invention relates to a process comprising the steps of:

(a) performing an assay method described hereinabove;

(b) identifying one or more ligands capable of binding to a ligand binding domain; and

(c) preparing a quantity of said one or more ligands.

Another aspect of the invention provides a process comprising the steps of:

(a) performing an assay method described hereinabove;

(b) identifying one or more ligands capable of binding to a ligand binding domain; and

(c) preparing a pharmaceutical composition comprising said one or more ligands.

Another aspect of the invention provides a process comprising the steps of:

(a) performing an assay method described hereinabove;

(b) identifying one or more ligands capable of binding to a ligand binding domain;

(c) modifying said one or more ligands capable of binding to a ligand binding domain;

(d) performing the assay method described hereinabove;

(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of proliferative disorders.

Preferably, said candidate compound is generated by conventional SAR modification of a compound of the invention.

As used herein, the term “conventional SAR modification” refers to standard methods known in the art for varying a given compound by way of chemical derivatisation.

The above methods may be used to screen for a ligand useful as an inhibitor of the interaction between HDM2 and p53.

Chemical Synthesis

The compounds of the present invention can be prepared by methods known in the art. In particular, the condensation reaction between arylsulfonyl halides and arylamines can be applied. N-substituted bisarylsulfonamides can be obtained by using secondary arylamines in this reaction or through alkylation of primary bisarylsulfonamide precursors. Alkylation can be achieved with alkyl or aralkyl halides by sulfonylamino-de-halogenation or with alkyl or aralkyl alcohols by e.g. the Tsunoda reaction [Tsunoda T, Otsuka J, Yamamiya Y, Ito S; Chem. Lett. 1994: 539-542]. A number of the bisarylsulfonamides of the present invention contain the thiophenesulfonyl function; appropriate thiophenesulfonyl halide precursors for the sulfonamide condensation reaction can be prepared as described [Cremlyn R J, Goulding K H, Swinbourne F J, Yung K-M; Phosphorus Sulfur 1981; 10: 111-119; Obafemi C A; Phosphorus Sulfur 1982; 13: 119-131.].

The present invention is further described by way of the following non-limiting examples and with reference to the following figures, wherein:

### BRIEF DESCRIPTION OF THE DRAWINGS

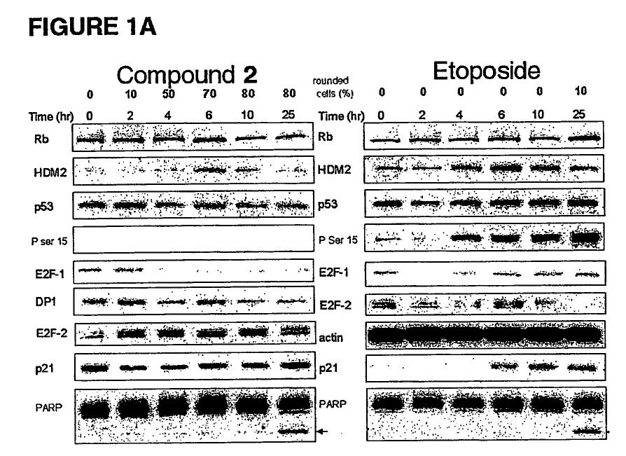

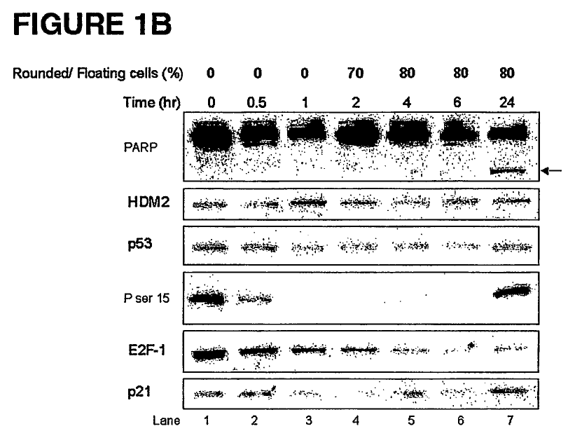

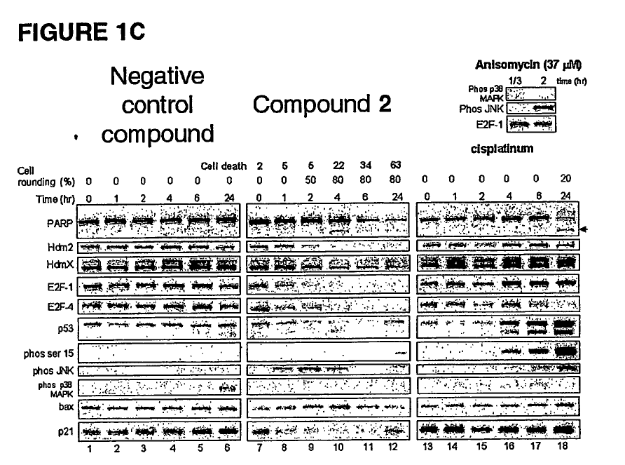

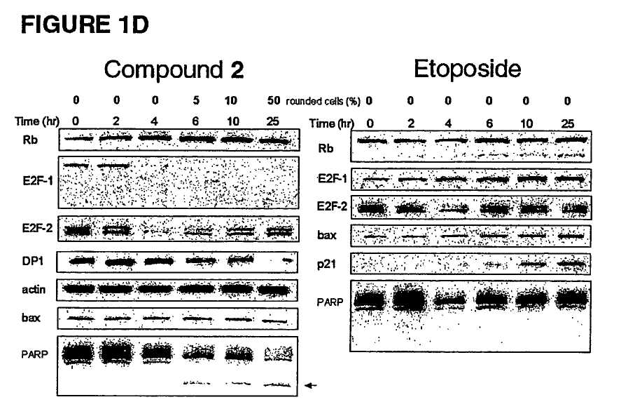

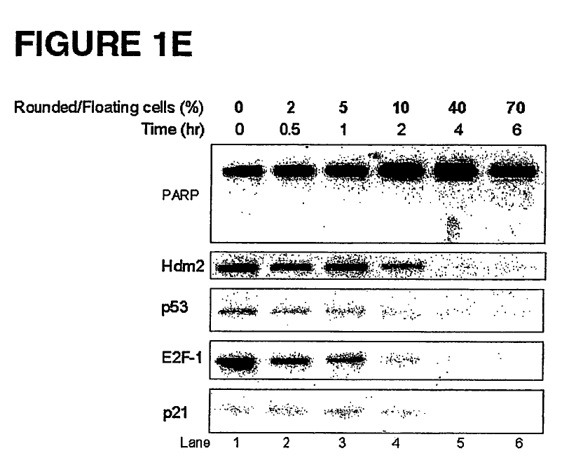

FIG. 1 shows the effect of compound 2 or DNA damage inducing agents on the levels of key proteins in the HDM2 pathway and cell morphology in a range of cell lines. In more detail, FIG. 1 shows: (A) The effect of compound 2 or etoposide on the induction of apoptosis proteins in AGS cells. AGS were treated with 5 times [IC₅₀] of either compound 2 or etoposide and collected at the indicated time points. Equal amounts of cell lysate were separated by SDS-PAGE and analysed by immunoblotting using the indicated antibodies. An estimation of the extent of cell rounding is indicated above each lane. The arrow indicates the position of a 85-kDa cleavage product of PARP, indicative of apoptosis. (B) The effect of compound 2 on the levels of the cell cycle proteins in AGS cells (wild-type 53). AGS cells were treated with 5 times [IC₅₀] of either compound 2 or etoposide and collected at the indicated time points. Equal amounts of cell lysate were separated by SDS-PAGE and analysed by immunoblotting using the indicated antibodies. An estimation of the extent of cell rounding is indicated above each lane. (C) The effect of negative control compound (refer Example 10), compound 2, cisplatin, and anisomycin on the levels of key proteins in MCF7 cells. MCF7 cells were treated with either 13.5 μM of negative control compound or compound 2 at 5 times [IC50]. Cisplatinum and anisomycin (a transcription inhibitor) treatments were with 50 μM or 37 μM, respectively. Cells were harvested at various time points; afterwards lysates were prepared and separated by SDS-PAGE and analysed by immunoblotting. The proteins detected are indicated. The extent of rounded or floating cells and the percentage viable cells were assessed. (D) The effect of compound 2 or etoposide on the levels of proteins and cell morphology in H1299 cells (p53 null cells). H1299 were treated with 5 times [IC₅₀] of either compound 2 or etoposide and collected at the indicated time points. Equal amounts of cell lysate were separated by SDS-PAGE and analysed by immunoblotting using a range of antibodies. An estimation of the extent of cell rounding is indicated above each lane. (E) Effect of 5 times [IC₅₀] of compound 2 on the levels of the cell cycle proteins in SJSA1 cells (overexpress HDM2). SJSA1 cells were treated with 5 times [IC₅₀] of compound 2 and collected at the indicated time points. Equal amounts of cell lysate were separated by SDS-PAGE and analysed by immunoblotting using a range of antibodies. An estimation of the extent of cell rounding is indicated above each lane.

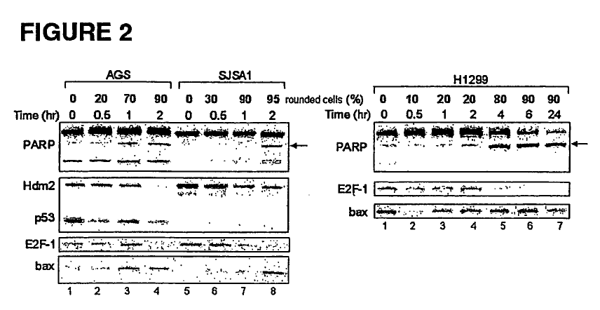

FIG. 2 shows the effect of compound 49 on AGS, SJSA1 and H1299 cells. Cells were treated with 5 times [IC₅₀] for each cell line and collected at the indicated time points. Equal amounts of cell lysate were separated by SDS-PAGE and analysed by immunoblotting using a range of antibodies. An estimation of the extent of cell rounding is indicated above each lane. The arrow indicates the position of an 85-kDa cleavage product of PARP, indicative of apoptosis.

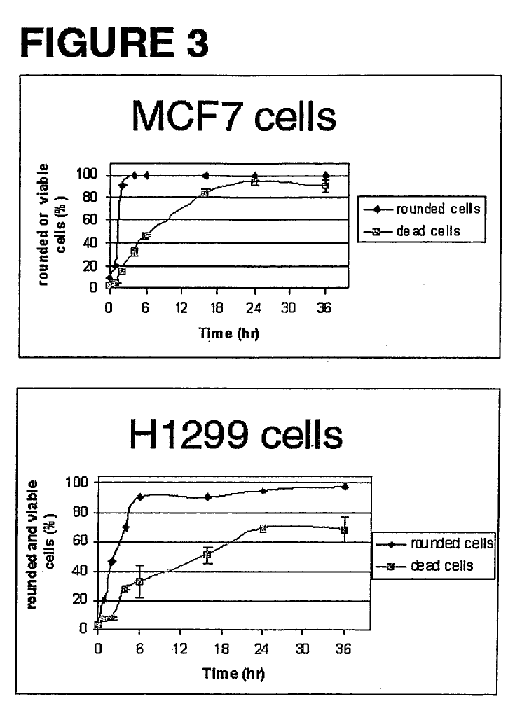

FIG. 3 shows the effect of compound 2 on the morphology and cell viability of MCF7 and H1299 cells. Cells were treated with 5 times [IC₅₀] of compound 2 and collected at various time points. The percentage of rounded cells was counted before cells were trypsinised and counted for cell viability using trypan blue.

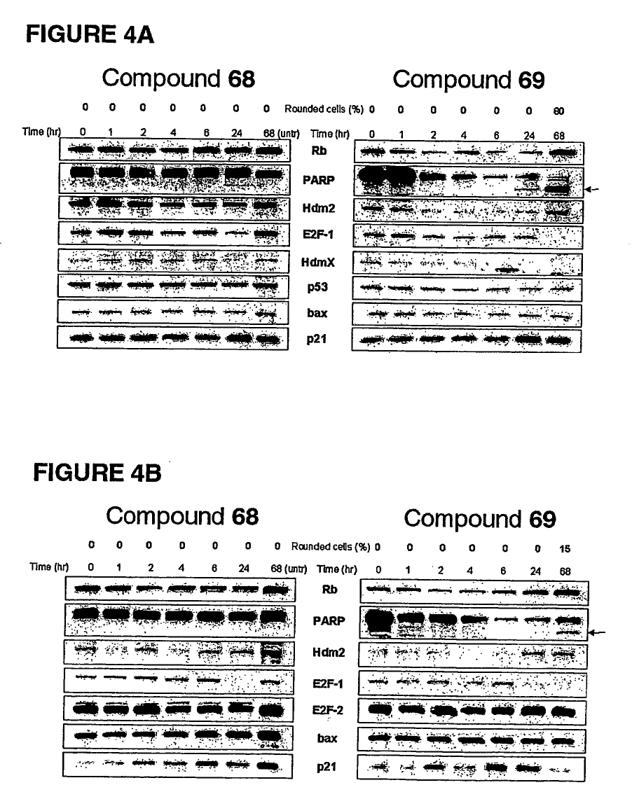

FIG. 4 shows the effect of compounds 68 and 69 on MCF7 cells and H1299 cells. In more detail: (A) MCF7 cells were treated with 5 times [IC₅₀] of compound 69 and the same concentration of 68 and collected at the indicated time points. Equal amounts of cell lysate were separated by SDS-PAGE and analysed by immunoblotting using a range of antibodies. An estimation of the extent of cell rounding is indicated above each lane. The arrow indicates the position of an 85-kDa cleavage product of PARP, indicative of apoptosis. (B) H1299 cells were treated with 5 times [IC₅₀] of compound 69 and the same concentration of 68 and collected at the indicated time points and analysed.

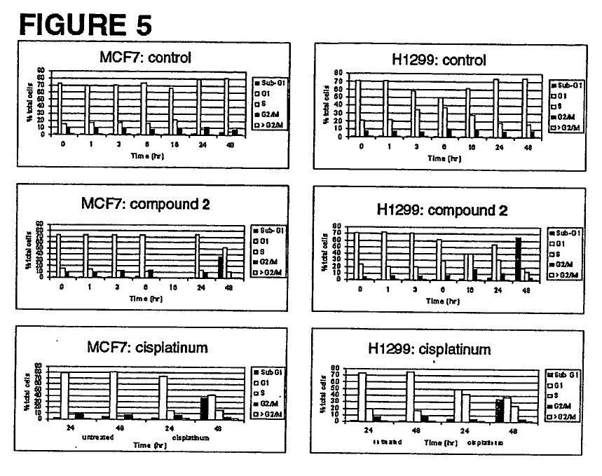

FIG. 5 shows the effect of compound 2 and cisplatinum on the cell cycle distribution of MCF7 and H1299 cells. Cells were left untreated, or treated with 5 times [IC₅₀] of either compound 2 or cisplatinum and collected at the indicated time points. Cells were stained with propidium iodide and the cell cycle position was determined using a flow cytometer.

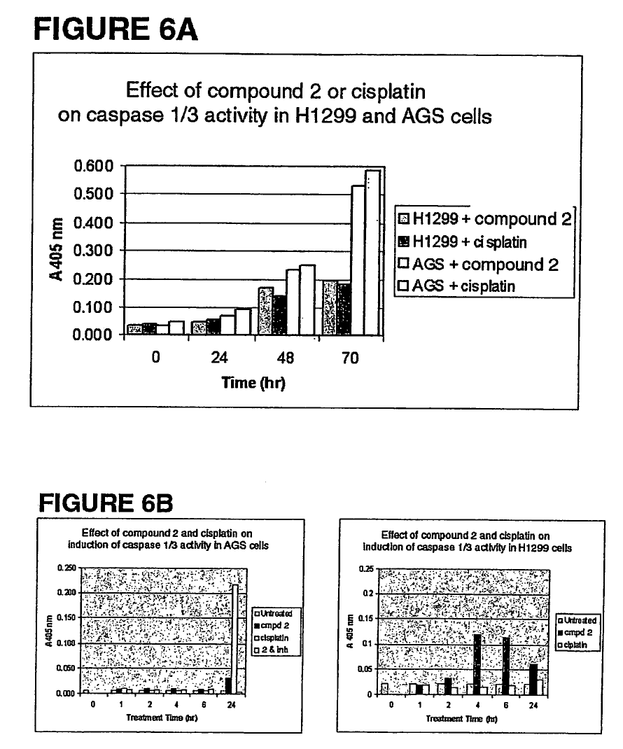

FIG. 6 shows the effect of compound 2 on the caspase activation in AGS and H1299 cells. In more detail: (A) Cells were treated with 3 times [IC₅₀] of compound 2 or cisplatinum and collected at the time points indicated. Cell lysates were prepared, the caspase activity was determined using the casp ACE assay and values were plotted on a graph. (B) Cells were treated with 5 times [IC₅₀] of compound 2 or cisplatinum and collected at the time points indicated. AGS cells were also incubated with compound 2 in the presence of a caspase inhibitor Z-VAD.fmk. Cell lysates were prepared, the caspase activity was determined using the casp ACE assay and values were plotted on a graph.

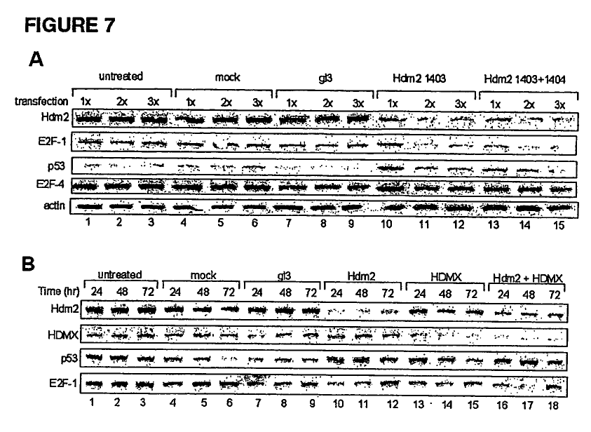

FIG. 7 shows the effect of HDM2 and HDMX siRNA on the levels of p53 and E2F-1 in MCF7 cells. In more detail: (A) MCF7 cells were untreated (lanes 1-3) mock transfected (lanes 4-6), transfected with gl3 control siRNA (lanes 7-9), HDM2 siRNA 1403 (lanes 10-12), or HDM2 siRNA 1403 and 1404 (lanes 13-15). This was repeated every 24 hours. Cells were collected 24 hours after each transfection so that cells had either been transfected once, twice, or three times and analysed by Western blotting. (B) MCF7 cells were untreated (lanes 1-3) mock transfected (lanes 4-6), transfected with g13 control siRNA (lanes 7-9), HDM2 siRNA 1403 (lanes 10-12), HDMX siRNA (lanes 13-15) or HDM2 and HDMX siRNA(lanes 16-18). Cells were collected at 24, 48, and 72 hours and analysed by Western blotting.

### EXAMPLES

General

HPLC retention times (t_{R}) were measured using Vydac 218TP54 columns (C₁₈ reversed-phase stationary phase; 4.5×250 mm columns), eluted at 1 mL/min with a linear gradient of acetonitrile in water (containing 0.1% CF₃COOH) as indicated, followed by isocratic elution. HPLC Method B refers to the following: Supercosil ABZ+Plus column (21.2×250 mm), eluted at 20 mL/min using a linear gradient of acetonitrile in water from 5 to 95% over 10 min, followed by isocratic elution. UV monitors (254 nm) were used. All purification work, unless otherwise stated, was performed using silica gel 60A (particle size 35-70 micron) eluting with hexane/EtOAc (4:1). Thin layer chromatography (TLC) was performed using aluminium sheets precoated with 0.2 mm silica gel 60 F₂₅₄. ¹H NMR spectra were recorded using various different instruments. Chemical shifts are given in ppm using TMS as standard and coupling constants (J) are stated in Hz. Mass spectra were recorded under positive or negative ion electrospray conditions.

### Example 1

General Method for the Preparation of Primary Bisarylsulfonamides

The appropriate sulfonyl chloride (1.0 mol eq) was suspended in dichloromethane. The suspension was cooled to 0° C. While stirring the reaction mixture, the appropriate amino component (aniline, benzylamine, etc., as appropriate) (1.1 mol eq) and pyridine (1.5 mol eq) were added. The reaction mixture was slowly warmed to room temperature. Stirring was continued until TLC [heptane:ethyl acetate (2:1)] indicated that the reaction had gone to completion. The product was isolated and purified as follows: the reaction mixture was diluted with dichloromethane and was washed successively with dilute aq HCl, saturated aq NaHCO₃, water, and brine. The organic fraction was dried over MgSO₄ and was concentrated under reduced pressure to yield the crude sulfonamide. Column chromatography [column (Isolute SI; Jones Chromatography), heptane:ethyl acetate (12:1→3:1)] afforded the desired bisarylsulfonamide.

The primary bisarylsulfonamide compounds of this invention were prepared in this manner. Analytical details for representative compounds are as follows:

5-Chloro-4-nitro-thiophene-2-sulfonic acid (4-chloro-phenyl)-amide 2. TLC R_{F}=0.72 (heptane:ethyl acetate, 2:1); HPLC t_{R} 17.08 min (20-80%, 20 min); ¹H NMR (CDCl₃) δ: 6.64 (1H, br s, NH), 7.05 (2H, d, J 8.5, Ph-H), 7.27 (2H, d, J 8.5, Ph-H), 7.84 (1H, s, thiophene-H); MS 352.94 (M−H)⁻, C₁₀H₆Cl₂N₂O₄S₂=353.20.

5-Chloro-4-nitro-thiophene-2-sulfonic acid (4-fluoro-phenyl)-amide 3. TLC R_{F}=0.35 (heptane:ethyl acetate, 2:1); ¹H NMR (CDCl₃) δ: 6.83 (1H, br s, NH), 6.99 (2H, t, J 8.5, 9.0, Ph-H), 7.10 (2H, dd, J 8.5, 9.0 Ph-H), 7.82 (1H, s, thiophene-H).

5-Chloro-4-nitro-thiophene-2-sulfonic acid (4-trifluoromethyl-phenyl)-amide 6. TLC R_{F}=0.45 (heptane:ethyl acetate, 2:1); ¹H NMR (CD₃OD) δ: 7.37 (2H, d, J 8.5, Ph-H), 7.62 (2H, d, J 8.5, Ph-H), 8.01 (1H, s, thiophene-H).

5-Chloro-thiophene-2-sulfonic acid (4-trifluoromethyl-phenyl)-amide 8. TLC R_{F}=0.44 (hexane:EtOAc, 4:1); ¹H NMR (CDCl₃) δ: 6.86 (d, 1H, J 4.1, thiophene-H), 6.95 (s, 1H, NH), 7.24 (d, 2H, J 8.4, Ph-H), 7.36 (d, 1H, J 4. 1, thiophene-H), 7.57 (d, 2H, J 8.4, Ph-H); MS 341 [M]⁺, C11H7ClF3NO2S2=341.76.

4,5-Dibromo-thiophene-2-sulfonic acid (3,5-bis-trifluoromethyl-phenyl)-amide 9. HPLC t_{R} 7.48 min (method B); MS 532.1 (M−H)⁻⁷⁹Br⁸¹Br, C₁₂H₅Br₂F₆NO₂S₂=533.10.

5-Chloro-thiophene-2-sulfonic acid (4-chloro-phenyl)-amide 10. HPLC t_{R} 8.45 min (method B); MS 306.1 (M−H)⁻³⁵Cl₂, C₁₀H₇Cl₂NO₂S₂=308.20.

5-Chloro-thiophene-2-sulfonic acid (3,5-bis-trifluoromethyl-phenyl)-amide 11. HPLC t_{R} 7.31 min (method B);); ¹H NMR (CDCl₃) δ: 6.85 (d, 1H, J 4.1, thiophene-H), 7.31 (s, 1H, NH), 7.34 (d, 1H, J 4.1, thiophene-H), 7.54 (s, 2H, Ph-H), 7.60 (s, 1H, Ph-H); MS 408.0 (M−H)⁻³⁵Cl, C₁₂H₆ClF₆NO₂S₂=409.76.

5-(2-Methylsulfanyl-pyrimidin-5-yl)-thiophene-2-sulfonic acid (3,5-dichloro-phenyl)-amide 12. HPLC t_{R} 9.01 min (method B); MS 429.9 (M−H)⁻³⁵Cl₂, Cl₅H₁₁Cl₂N₃O₂S₃=432.3677.

4-Oxazol-2-yl-N-(4-trifluoromethyl-phenyl)-benzenesulfonamide 13. HPLC t_{R} 9.02 min (method B); MS 367.1 (M−H)⁻, C₁₆H₁₁F₃N₂O₃S=368.33.

N-(3,5-Bis-trifluoromethyl-phenyl)-4-oxazol-2-yl-benzenesulfonamide 14. HPLC t_{R} 9.13 min (method B); MS 435.1 (M−H)⁻, C₁₇H₁₀F₆N₂O₃S=436.33.

4-Bromo-5-chloro-thiophene-2-sulfonic acid (4-trifluoromethyl-phenyl)-amide 15. HPLC t_{R} 8.25 min (method B); MS 419.9 (M−H)⁻⁸¹Br³⁵Cl, C₁₁H₆BrClF₃NO₂S₂=420.65.

5-Bromo-thiophene-2-sulfonic acid (4-chloro-phenyl)-amide 16. HPLC t_{R} 9.57 min (method B); MS 352.0 (M−H)⁻⁸¹Br³⁵Cl, C₁₀H₇BrClNO₂S₂=352.66.

5-Bromo-thiophene-2-sulfonic acid (3,5-dichloro-phenyl)-amide 17. HPLC t_{R} 9.11 min (method B); MS 385.8 (M−H)⁻⁸¹Br³⁵Cl₂, C₁₀H₆BrCl₂NO₂S₂=387.10.

5-Bromo-thiophene-2-sulfonic acid (3,5-bis-trifluoromethyl-phenyl)-amide 18. HPLC t_{R} 8.07 min (method B); MS 453.9 (M−H)⁻⁸¹Br, C₁₂H₆BrF₆NO₂S₂=454.21.

N-(4-Chloro-phenyl)-3-nitro-benzenesulfonamide 19. HPLC t_{R} 9.06 min (method B); MS 311.0 (M−H)⁻³⁵Cl, C₁₂H₉ClN₂O₄S=312.73.

3-Nitro-N-(4-trifluoromethyl-phenyl)-benzenesulfonamide 20. MS 345.0 (M−H)⁻, C₁₃H₉F₃N₂O₄S=346.28.

N-(3,5-Bis-trifluoromethyl-phenyl)-3-nitro-benzenesulfonamide 21. MS 413.0 (M−H)⁻, C₁₄H₈F₆N₂O₄S=414.29.

N-(2,4-Dichloro-phenyl)-3-nitro-benzenesulfonamide 22. MS 344.8 (M−H)⁻³⁵Cl₂, C₁₂H₈Cl₂N₂O₄S=347.17.

5-Benzenesulfonyl-thiophene-2-sulfonic acid (4-trifluoromethyl-phenyl)-amide 23. MS 446.0 (M−H)⁻, C₁₇H₁₂F₃NO₄S₃=447.47.

5-Benzenesulfonyl-thiophene-2-sulfonic acid (4-chloro-phenyl)-amide 24. MS 411.9 (M−H)⁻³⁵Cl, C₁₆H₁₂ClNO₄S₃=413.92.

5-Benzenesulfonyl-thiophene-2-sulfonic acid (3,5-dichloro-phenyl)-amide 25. MS 446.0 (M−H)⁻³⁵Cl₂, C₁₆H₁₁Cl₂NO₄S₃=448.36.

5-Chloro-thiophene-2-sulfonic acid (3,4-dichloro-phenyl)-amide 26. MS 339.8 [M−H]⁻³⁵Cl₃, C₁₀H₆Cl₃NO₂S₂=342.65.

4,5-Dibromo-thiophene-2-sulfonic acid (3-trifluoromethyl-phenyl)-amide 27. ¹H NMR (CDCl₃) δ: 6.69 (s, 1H, NH), 7.26 (s, 1H, Ph-H), 7.28 (m, 1H, Ph-H), 7.33 (s, 1H, thiophene-H), 7.42 (d, 2H, J 4.2, Ph-H); MS 463.8 (MH)⁻⁷⁹Br⁸¹Br, C₁₁H₆Br₂F₃NO₂S₂=465.10.

4,5-Dibromo-thiophene-2-sulfonic acid (3,4-dichloro-phenyl)-amide 28. MS 465.7 (M−H)⁻⁸¹Br⁷⁹Br³⁷Cl³⁵Cl₂, C₁₀H₅Br₂Cl₂NO₂S₂=466.00.

N-(3,5-Bis-trifluoromethyl-phenyl)-4-chloro-3-nitro-benzenesulfonamide 29. TLC R_{F}=0.78 (heptane:ethyl acetate, 2:1); ¹H NMR (CDCl₃) δ: 7.53 (3H, s, Ph-H), 7.60 (1H, d, J 8.5, Ph-H), 7.85 (1H, m, Ph-H) 8.30 (1H, s, Ph-H); MS 447.0 (M−H)⁻³⁵Cl, C₁₄H₇ClF₆N₂O₄S=448.72.

4-Chloro-N-(3,4-dichloro-phenyl)-3-nitro-benzenesulfonamide 30. MS 380.9 (M−H⁻³⁷Cl³⁵Cl₂, C₁₂H₇Cl₃N₂O₄S=381.62.

5-(1-Methyl-5-trifluoromethyl-1H-pyrazol-4-yl)-thiophene-2-sulfonic acid (4-trifluoromethylphenyl)-amide 31. TLC R_{F}=0.39 (heptane:ethyl acetate, 2:1); HPLC t_{R} 22.14 min (10-70%, 20 min); ¹H NMR (CD₃OD) δ: 3.915 (3H, s, NCH₃), 6.60 (1H, s, diazole-H), 7.06 (1H, d, J 3.5, thiophene-H), 7.11 (1H, br s, NH), 7.23 (2H, d, J 8.0, Ph-H), 7.52 (3H, m, Ph-H & thiophene-H); MS 456.24 (M+H)⁺, C₁₆H₁₁F₆N₃O₂S₂=455.40.

5-Chloro-thiophene-2,4-disulfonic acid bis-[(4-fluoro-phenyl)-amide] 32. HPLC t_{R} 19.68 min (10-70%, 20 min); ¹H NMR (CDCl₃) δ: 6.88 (4H, m, Ph-H), 6.97 (4H, m, Ph-H), 7.47 (1H, s, thiophene-H); MS 462.95 (M−2H)⁻, C₁₆H₁₁ClF₂N₂O₄S₃=464.92.

5-Chloro-thiophene-2,4-disulfonic acid bis-[(4-trifluoromethyl-phenyl)-amide] 33. HPLC t_{R} 22.54 min (10-70%, 20 min); ¹H NMR (CDCl₃) δ: 7.09 (2H, d, J 8.5, Ph-H), 7.14 (2H, d, J 8.5, Ph-H), 7.40 (2H, d, J 9.0, Ph-H), 7.46 (2H, d, J 9.0, Ph-H), 7.66 (1H, s, thiophene-H); MS 562.88 (M−2H)⁻, C₁₈H₁₁ClF₆N₂O₄S₃=564.93.

4-Methyl-3-nitro-N-(4-trifluoromethyl-phenyl)-benzenesulfonamide 34. HPLC t_{R} 20.32 min (10-70%, 20 min); ¹H NMR (CDCl₃) δ: 2.90 (3H, s, CH₃), 7.15 (2H, d, J 8.0, Ph-H), 7.38 (1H, d, J 8.5, Ph-H), 7.41 (2H, d, J 8.0, Ph-H), 7.81 (1H, d, J 8.5, Ph-H), 8.38 (1H, s, Ph-H); MS 359.11 (M−H)⁻, C₁₄H₁₁F₃N₂O₄S=360.31.

4-Chloro-3-nitro-N-(4-trifluoromethyl-phenyl)-benzenesulfonamide 35. TLC R_{F}=0.40 (heptane:ethyl acetate, 2:1); HPLC t_{R} 15.45 min (20-80%, 20 min); ¹H NMR (CDCl₃) δ: 7.16 (2H, d, J 8.5, Ph-H), 7.43 (2H, d, J 8.5, Ph-H), 7.57 (1H, d, J 8.5, Ph-H), 7.83 (1H, dd, J 8.5, 8.5, Ph-H), 8.29 (1H, d, J 2.5, Ph-H).

3-Amino-4-methyl-N-(4-trifluoromethyl-phenyl)-benzenesulfonamide 36. HPLC t_{R} 17.14 min (10-70%, 20 min); ¹H NMR (CD₃OD) δ: 2.07 (3H, s, CH₃), 7.00 (3H, m, Ph-H), 7.11 (2H, d, J 8.0, Ph-H), 7.37 (2H, d, J 8.0, Ph-H); MS 329.15 (M−H)⁻, C₁₄H₁₃F₃N₂O₂S=330.33.

N-(4-Chloro-phenyl)-4-methyl-3-nitro-benzenesulfonamide 37. ¹H NMR (CDCl₃) δ: 2.58 (3H, s, CH₃), 6.72 (1H, br s, NH), 6.96 (2H, d, J 8.0, Ph-H), 7.17 (2H, d, J 8.0, Ph-H), 7.39 (1H, d, J 8.0, Ph-H), 7.74 (1H, d, J 8.0, Ph-H), 8.31 (1H, s, Ph-H).

4-Chloro-N-(4-chloro-phenyl)-3-nitro-benzenesulfonamide 38. ¹H NMR (CDCl₃) δ: 6.74 (1H, br s, NH), 6.98 (2H, d, J 8.0, Ph-H), 7.19 (2H, d, J 8.0, Ph-H), 7.57 (1H, d, J 8.0, Ph-H), 7.71 (1H, d, J 8.0, Ph-H), 8.20 (1H, s, Ph-H).

5-Chloro-4-nitro-thiophene-2-sulfonic acid (3,5-dichloro-phenyl)-amide 39. TLC R_{F}=0.75 (heptane:ethyl acetate, 2:1); HPLC 18.83 min (20-80%, 20 min); ¹H NMR (CDCl₃) δ: 6.97 (1H, br s, NH), 7.05 (2H, m, Ph-H), 7.17 (1H, m, Ph-H), 7.92 (1H, s, thiophene-H). MS 386.84 (M−H)⁻, C₁₀H₅Cl₃N₂O₄S₂=387.65.

5-Chloro-4-nitro-thiophene-2-sulfonic acid (3,5-difluoro-phenyl)-amide 40. HPLC t_{R} 16.59 min (20-80%, 20 min); ¹H NMR (CDCl₃) δ: 6.63 (1H, m, Ph-H), 6.70 (2H, m, Ph-H), 7.01 (1H, br s, NH), 7.93 (1H, s, thiophene-H). MS 353.96 (M−H)⁻, C₁₀H₅ClF₂N₂O₄S₂=354.74.

5-Bromo-6-chloro-pyridine-3-sulfonic acid (4-trifluoromethyl-phenyl)-amide 41. TLC R_{F}=0.75 (heptane:ethyl acetate, 2:1); HPLC t_{R} 21.09 min (10-70%, 20 min); ¹H NMR (CDCl₃) δ: 7.17 (2H, d, J 8.5, Ph-H), 7.47 (2H, d, J 8.5, Ph-H), 8.23 (1H, m, pyridine-H), 8.62 (1H, m, pyridine-H); MS 414.88 (M−H)⁻, C₁₂H₇BrClF₃N₂O₂S=415.61.

5-Bromo-6-chloro-pyridine-3-sulfonic acid (3,5-bis-trifluoromethyl-phenyl)-amide 42. HPLC t_{R} 23.17 min (10-70%, 20 min); ¹H NMR (CDCl₃) δ: 7.54 (3H, s, Ph-H), 8.26 (1H, m, pyridine-H), 8.65 (1H, m, pyridine-H); MS 482.80 (M−H)⁻, C₁₃H₆BrClF₆N₂O₂S=483.61.

5-(1-Methyl-5-trifluoromethyl-1H-pyrazol-4-yl)-thiophene-2-sulfonic acid (3,5-bis-trifluoro-methyl-phenyl)-amide 43. TLC R_{F}=0.63 (heptane:ethyl acetate, 2:1); ¹H NMR (CD₃OD) δ: 3.97 (3H, s, NCH₃), 6.82 (1H, s, diazole-H), 7.37 (1H, dd, J 4.0, 4.0, thiophene-H), 7.64 (1H, dd, J 4.0, 4.0, thiophene-H), 7.68 (1H, s, Ph-H), 7.73 (2H, s, Ph-H).

5-Chloro-4-nitro-thiophene-2-sulfonic acid (3,5-bis-trifluoromethyl-phenyl)-amide 44. HPLC t_{R} 19.49 min (20-80%, 20 min); ¹H NMR (CD₃OD) δ: 7.19 (1H, s, Ph-H), 7.58 (2H, s, Ph-H), 7.67 (1H, br s, NH), 7.95 (1H, s, thiophene-H); MS 452.51 (M−2H)⁻, C₁₂H₅ClF₆N₂O₄S₂=454.75.

5-Chloro-4-nitro-thiophene-2-sulfonic acid 4-fluoro-benzylamide 45. HPLC t_{R} 15.89 min (20-80%, 20 min); TLC R_{F}=0.64 (heptane:ethyl acetate, 2:1); ¹H NMR (CDCl₃) δ: 4.20 (2H, d, J 6.0, ArCH₂), 6.95 (2H, t, J 8.5, Ph-H), 7.17 (2H, dd, J 8.5, 8.5, Ph-H), 7.82 (1H, s, thiophene-H); MS 348.96 (M−2H)⁻, C₁₁H₈ClFN₂O₄S₂=350.78

5-Chloro-4-nitro-thiophene-2-sulfonic acid 4-trifluoromethyl-benzylamide 46. TLC R_{F}=0.71 (heptane:ethyl acetate, 2:1); HPLC t_{R} 15.16 min (20-80%, 20 min); ¹H NMR (CDCl₃) δ: 4.30 (2H, d, J 6.0, ArCH₂), 5.08 (1H, br s, NH), 7.34 (2H, d, J 8.0, Ph-H), 7.54 (2H, d, J 8.0, Ph-H), 7.86 (1H, s, thiophene-H); MS 398.91 (M−2H)⁻, C₁₂H₈ClF₃N₂O₄S₂=400.78.

4-Chloro-N-(3,5-dichloro-phenyl)-3-nitro-benzenesulfonamide 47. HPLC t_{R} 18.64 min (20-80%, 20 min); ¹H NMR (CDCl₃) δ: 6.98 (2H, m, Ph-H), 7.01 (1H, m, Ph-H), 7.60 (1H, d, J 8.5, Ph-H), 7.84 (1H, dd J 2.0, 8.5, Ph-H), 8.27 (1H, d, J 2.0, Ph-H); MS 380.91 (M−H)⁻, C₁₂H₇Cl₃N₂O₄S=381.62.

5-Chloro-4-nitro-thiophene-2-sulfonic acid [2-(1H-indol-3-yl)-ethyl]-amide 48. TLC R_{F}=0.44 (heptane:ethyl acetate, 2:1); HPLC t_{R} 13.48 min (20-80%, 20 min); ¹H NMR (CD₃OD) δ: 2.91 (2H, t, J 6.5, CH₂), 3.43 (2H, t, J 6.5, CH₂), 6.89 (1H, t, J 8.0, indole-H), 6.97 (1H, t, J 8.0, indole-H), 7.01 (1H, s, indole-H), 7.23 (1H, d, J 8.0, indole-H), 7.38 (1H, d, J 8.0, Ar), 7.47 (1H, s, thiophene-H); MS 384.21 (M−H)⁻, C₁₄H₁₂ClN₃O₄S₂=385.85.

5-Chloro-4-nitro-thiophene-2-sulfonic acid [2-(1H-indol-3-yl)-1-methyl-ethyl]-amide 49. TLC R_{F}=0.49 (heptane:ethyl acetate, 2:1); HPLC t_{R} 13.48 min (20-80%, 20 min); ¹H NMR (CDCl₃) δ: 1.40 (3H, d, J 6.5, CHCH₃), 2.58 (1H, dd, J 10.5, 10.5, CH₂), 2.96 (1H, dd, J 3.5, 3.5, CH₂), 3.64 (1H, m, CHCH₃), 6.91 (2H, m, indole-H), 7.04 (1H, t, J 8.0, 7.0, indole-H), 7.17 (1H, d, J 8.0, indole-H), 7.25 (2H, m, indole-H & thiophene-H), 8.105 (1H, br s, NH); MS 398.63 (M−H)⁻, C₁₅H₁₄ClN₃O₄S₂=399.87.

5-Chloro-4-nitro-thiophene-2-sulfonic acid 3,5-dichloro-benzylamide 54. HPLC t_{R} 18.14 min (20-80%, 20 min); ¹H NMR (CDCl₃) δ: 4.20 (2H, d, J 6.0, ArCH₂), 5.16 (1H, br s, NH), 7.07 (2H, s, Ph-H), 7.23 (1H, s, Ph-H), 7.84 (1H, s, thiophene-H); MS 400.86 (M−H)⁻, C₁₁H₇Cl₃N₂O₄S₂=401.67.

5-Chloro-4-nitro-thiophene-2-sulfonic acid 3,5-difluoro-benzylamide 55. HPLC t_{R} 16.42 min (20-80%, 20 min); ¹H NMR (CDCl₃) δ: 4.21 (2H, d, J 6.0, ArCH₂), 5.20 (1H, br s, NH), 6.69 (1H, m, Ph-H), 6.75 (2H, m, Ph-H), 7.87 (1H, s, thiophene-H). MS 367.16 (M−H)⁻, C₁₁H₇ClF₂N₂O₄S₂=368.77.

5-Chloro-4-nitro-thiophene-2-sulfonic acid 4-chloro-benzylamide 56. HPLC t_{R} 16.82 min (20-80%, 20 min); ¹H NMR (CDCl₃) δ: 4.20 (2H, d, J 6.0, ArCH₂), 5.13 (1H, br s, NH), 7.07 (2H, d, J 8.5, Ph-H), 7.23 (2H, d, J 8.5, Ph-H), 7.82 (1H, s, thiophene-H); MS 366.95 (M−H)⁻, C₁₁H₈Cl₂N₂O₄S₂=367.23.

5-Chloro-4-nitro-thiophene-2-sulfonic acid [1-(4-fluoro-phenyl)-ethyl]-amide 57. HPLC t_{R} 16.25 min (20-80%, 20 min); ¹H NMR (CDCl₃) δ: 1.45 (3H, d, J 7.0, CH₃), 4.54 (1H, m, Ph CH), 5.18 (1H, br s, NH), 6.90 (2H, m, Ph-H), 7.10 (2H, m, Ph-H), 7.55 (1H, s, thiophene-H); MS 363.18 (M−H)⁻, C₁₂H₁₀ClFN₂O₄S₂=364.80.

5-Chloro-4-nitro-thiophene-2-sulfonic acid (4-chloro-phenyl)-isobutyl-amide 58. HPLC t_{R} 20.04 min (20-80%, 20 min); ¹H NMR (CDCl₃) δ: 0.85 (6H, d, J 7.0, CH₃), 1.53 (1H, m, CH(CH₃)₂), 5.18 (2H, d, J 7.5, NCH₂), 7.08 (2H, d, J 8.5, Ph-H), 7.26 (2H, d, J 8.5, Ph-H), 7.61 (1H, s, thiophene-H).

5-Chloro-4-nitro-thiophene-2-sulfonic acid (1H-benzoimidazol-2-yl)-amide 59. HPLC t_{R} 17.89 min (0-60%, 20 min); ¹H NMR (CDCl₃) δ: 7.10 (1H, t, J 8.0, benzimidazole-5/6), 7.23 (2H, m, benzimidazole-4/7 & 5/6), 7.63 (1H, d, J 8.5, benzimidazole-4/7), 8.15 (1H, s, thiophene-H); MS 359.03 (M+H)⁺, C₁₁H₇ClN₄O₄S₂=358.78.

5-Chloro-4-nitro-thiophene-2-sulfonic acid [2-(6-chloro-1H-indol-3-yl)-ethyl]-amide 60. HPLC t_{R} 23.16 min (0-60%, 20 min); ¹H NMR (CDCl₃) δ: 2.91 (2H, t, J 6.5, CH₂), 3.38 (2H, t, J 6.5, CH₂), 4.70 (1H, br s, NH), 6.89 (1H, t, J 8.0, indole-H), 6.95 (2H, m, indole-H), 7.23 (2H, m, indole-H), 7.59 (1H, s, thiophene-H), 8.06 (1H, br s, indole-NH); MS 419.94 (M−H)⁻, C₁₄H₁₁Cl₂N₃O₄S₂=420.29.

5-Chloro-4-nitro-thiophene-2-sulfonic acid (4-methoxy-phenyl)-amide 61. HPLC t_{R} 12.81 min (20-80%, 20 min); ¹H NMR (DMSO-d₆) δ: 3.70 (3H, s, OMe), 6.88 (2H, d, J 8.5, Ph-H), 7.07 (2H, d, J 8.5, Ph-H), 7.82 (1H, s, thiophene-H); MS 349.26 (M+H)⁺, C₁₁H₉ClN₂O₅S₂=348.78.

5-Chloro-4-nitro-thiophene-2-sulfonic acid phenylamide 62. HPLC t_{R} 15.36 min (20-80%, 20 min); ¹H NMR (CDCl₃) δ: 6.66 (1H, s, NH), 7.10 (2H, d, J 8.5, Ph-H), 7.20 (1H, m, Ph-H), 7.28 (2H, d, J 8.5, Ph-H), 7.82 (1H, s, thiophene-H); MS 317.22 (M−H)⁻, C₁₀H₇ClN₂O₄S₂=318.76.

5-Chloro-4-nitro-thiophene-2-sulfonic acid p-tolylamide 63. HPLC t_{R} 16.59 min (20-80%, 20 min); ¹H NMR (CDCl₃) δ: 2.27 (3H, s, CH₃), 6.54 (1H, br s, NH), 6.98 (2H, d, J 7.5, Ph-H), 7.08 (2H, d, J 7.5, Ph-H), 7.79 (1H, s, thiophene-H); MS 331.24 (M−H)⁻, C₁₁H₉ClN₂O₄S₂=332.78.

5-Chloro-4-nitro-thiophene-2-sulfonic acid benzylamide 64. HPLC t_{R} 15.73 min (20-80%, 20 min); ¹H NMR (CDCl₃) δ: 4.24 (2H, d, J 5.5, CH₂), 5.05 (1H, br s, NH), 7.16 (2H, m, Ph-H), 7.24 (3H, m, Ph-H), 7.76 (1H, s, thiophene-H); MS 331.04 (M−H)⁻, C₁₁H₈ClN₂O₄S₂=332.78.

5-Nitro-thiophene-2-sulfonic acid (4-chloro-phenyl)-amide 68. HPLC t_{R} 22.61 min (0-60%, 20 min); ¹H NMR (CDCl₃) δ: 7.11 (2H, d, J 9.0, Ph-H), 7.32 (2H, d, J 9.0, Ph-H), 7.38 (1H, d, J 6.0, thiophene 3/4), 7.95 (1H, d, J 6.0, thiophene 3/4); MS 317.02 (M−H)⁻, C₁₀H₇ClN₂O₄S₂=318.76.

4-Nitro-thiophene-2-sulfonic acid (4-chloro-phenyl)-amide 69. HPLC t_{R} 21.86 min (0-60%, 20 min); ¹H NMR (CDCl₃) δ: 7.11 (2H, d, J 9.0, Ph-H), 7.32 (2H, d, J 9.0, Ph-H), 8.00 (1H, s, thiophene 3/5), 8.43 (1H, s, thiophene 3/5). MS 317.08 (M−H)⁻, C₁₀H₇ClN₂O₄S₂=318.76.

5-Chloro-thiophene-2,4-disulfonic acid bis-[(4-chloro-phenyl)-amide] 70. HPLC t_{R} 19.66 min (0-60%, 20 min); ¹H NMR (CDCl₃) δ: 6.96 (4H, d, J 8.0, Ph-H), 7.04 (1H, m, NH), 7.19 (5H, m, Ph-H & NH), 7.57 (1H, s, thiophene-H). MS 496.83 (M−H)⁻, C₁₆H₁₁Cl₃N₂O₄S₃=497.83.

5-Ethyl-4-nitro-thiophene-2-sulfonic acid (4-chloro-phenyl)-amide 71. HPLC t_{R} 17.41 min (20-80%, 20 min); ¹H NMR (CDCl₃) δ: 1.36 (3H, t, J 7.0, CH₂CH₃), 3.28 (2H, dd, J 7.0, 7.0, CH₂H₃), 6.65 (1H, br s, NH), 7.10 (2H, d, J 8.0, Ph-H), 7.30 (2H, d, J 8.0, Ph-H), 7.96 (1H, s, thiophene-H); MS 345.05 (M−H)⁻, C₁₂H₁₁ClN₂O₄S₂=346.81.

Thioacetic acid S-[5-(4-chloro-phenylsulfamoyl)-3-nitro-thiophen-2-yl]ester 72. HPLC t_{R} 21.56 min (20-80%, 20 min); ¹H NMR (CD₃OD) δ: 2.59 (3H, s, CH₃), 7.15 (2H, d, J 8.5, Ph-H), 7.28 (2H, d, J 8.5, Ph-H), 7.91 (1H, s, thiophene-H); MS 391.15 (M−H)⁻, C₁₂H₉ClN₂O₅S₃=392.86.

5-Methyl-4-nitro-thiophene-2-sulfonic acid (4-chloro-phenyl)-amide 73. HPLC t_{R} 17.95 min (20-80%, 20 min); ¹H NMR (CD₃OD) δ: 2.66 (3H, s, CH₃), 7.13 (2H, d, J 8.0, Ph-H), 7.32 (2H, d, J 8.0, Ph-H), 7.70 (1H, s, thiophene-H); MS 333.50 (M−H)⁻, C₁₁H₉ClN₂O₄S₂=332.78.

5-Methyl-thiophene-2,4-disulfonic acid bis-[(4-chloro-phenyl)-amide] 74. HPLC t_{R} 22.19 min (20-80%, 20 min); ¹H NMR (CDCl₃) δ: 2.31 (3H, s, CH₃), 7.01 (2H, d, I 8.0, Ph-H), 7.10 (2H, d, J 8.0, Ph-H), 7.26 (4H, m, Ph-H), 7.81 (1H, s, thiophene-H); MS 476.94 (M−H)⁻, C₁₇H₁₄Cl₂N₂O₄S₃=477.41.

4-Nitro-thiophene-2-sulfonic acid (4-trifluoromethyl-phenyl)-amide 76. HPLC t_{R} 15.99 min (20-80%, 20 min); ¹H NMR (CDCl₃) δ: 7.28 (2H, d, J 8.5, Ph-H), 7.53 (2H, d, J 8.5, Ph-H), 8.03 (1H, s, thiophene 3/5), 8.41 (1H, s, thiophene 3/5); MS 351.18 (M−H)⁻, C₁₁H₇F₃N₂O₄S₂=352.31.

4-Nitro-thiophene-2-sulfonic acid [2-(1H-indol-3-yl)-ethyl]-amide 77. HPLC t_{R} 14.01 min (20-80%, 20 min); ¹H NMR (CDCl₃) δ: 2.97 (2H, t, J 6.5, CH₂), 3.41 (2H, t, J 6.5, CH₂), 7.01 (2H, m, indole-H), 7.13 (1H, t, J 7.5, indole-H), 7.30 (1H, d, J 7.5, indole-H), 7.38 (1H, d, J 7.5, indole-H), 7.75 (1H, s, thiophene 3/5), 8.16 (1H, s, thiophene 3/5); MS 350.24 (M−H)⁻, C₁₄H₁₃N₃O₄S₂=351.40.

5-Morpholin-4-yl-4-nitro-thiophene-2-sulfonic acid (4-chloro-phenyl)-amide 79. HPLC t_{R} 18.52 min (10-70%, 20 min); ¹H NMR (CDCl₃) δ: 3.33 (4H, t, J 5.0, morpholine-H), 3.88 (4H, t, J 5.0, morpholine-H), 7.03 (1H, br s, NH), 7.13 (2H, d, J 8.0, Ph-H), 7.29 (2H, d, J 8.0, Ph-H), 7.91 (1H, s, thiophene-H); MS 404.26 (M+H)⁺, C₁₄H₁₄ClN₃O₅S₂=403.86.

5-(2-Methoxy-ethylamino)-4-nitro-thiophene-2-sulfonic acid (4-chloro-phenyl)-amide 80. HPLC t_{R} 18.38 min (10-70%, 20 min); ¹H NMR (CDCl₃) δ: 3.33 (3H, s, OMe), 3.37 (2H, t, J 5.5, CH₂), 3.58 (2H, t, J 5.5, CH₂), 7.06 (2H, d, J 7.0, Ph-H), 7.21 (2H, d, J 7.0, Ph-H), 7.33 (1H, br s, NH), 7.70 (1H, s, thiophene-H), 8.47 (1H, br s, NH); MS 390.21 (M−H)⁻, C₁₃H₁₄ClN₃O₅S₂=391.85.

4-Chloro-N-[2-(5-chloro-1H-indol-3-yl)-ethyl]-3-nitro-benzenesulfonamide 81. HPLC t_{R} 16.63 min (20-80%, 20 min); ¹H NMR (CDCl₃) δ: 2.83 (2H, t, J 6.0, CH₂), 3.29 (2H, t, J 6.0, CH₂), 6.54 (1H, br s, NH), 6.90 (2H, m, indole-H), 7.23 (2H, m, indole-H), 7.34 (1H, dd, J 1.5, 8.0, Ph-H), 7.62 (1H, dd, J 1.5, 8.0, Ph-H), 8.15 (1H, d, J 1.5, Ph-H), 9.54 (1H, br s, indole NH); MS 412.19 (M−2H)⁻, C₁₆H₁₃Cl₂N₃O₄S=414.26.

N-[2-(5-Chloro-1H-indol-3-yl)-ethyl]-4-methyl-3-nitro-benzenesulonamide 82. HPLC t_{R} 16.25 min (20-80%, 20 min); ¹H NMR (CDCl₃) δ: 2.73 (3H, s, CH₃), 2.82 (2H, t, J 6.0, CH₂), 3.22 (2H, t, J 6.0, CH₂), 6.86 (1H, m, indole-H), 6.91 (1H, s, indole-H), 7.15 (2H, m, indole-H), 7.20 (1H, m, Ph-H), 7.63 (1H, dd, J 2.0, 8.0, Ph-H), 8.13 (1H, d, J 2.0, Ph-H); MS 392.03 (M−H)⁻, C₁₇H₁₆ClN₃O₄S=393.85.

N-(1H-Benzoimidazol-2-yl)-4-chloro-3-nitro-benzenesulfonamide 83. HPLC t_{R} 17.55 min (0-60%, 20 min); ¹H NMR (CDCl₃) δ: 7.07 (1H, t, J 8.0, Ph-H), 7.16 (1H, t, J 8.0, Ph-H), 7.22 (2H, d, J 9.0, Ph-H), 7.64 (2H, d, J 7.5, Ph-H), 7.97 (1H, dd, J 2.5, 9.0, Ph-H), 8.42 (1H, d, J 2.5, NH); MS 351.25 (M−H)⁻, C₁₃H₉ClN₄O₄S=352.75.

6-Chloro-imidazo[2,1-b]thiazole-5-sulfonic acid (3,5-bis-trifluoromethyl-phenyl)-amide 84. HPLC t_{R} 24.09 min (0-60%, 20 min); ¹H NMR (CDCl₃) δ: 3.40 (1H, br s, NH), 7.10 (1H, d, J 4.5, thiazole-H), 7.54 (1H, s, Ph-H), 7.56 (2H, s, Ph-H), 7.90 (1H, d, J 4.5, thiazole-H); MS 447.86 (M−2H)⁻, C₁₃H₆ClF₆N₃O₂S₂=449.78.

2,3-Dihydro-benzo[1,4]dioxine-6-sulfonic acid (4-chloro-phenyl)-amide 85. HPLC t_{R} 21.20 min (0-60%, 20 min); ¹H NMR (CDCl₃) δ: 4.27 (4H, m, CH₂), 6.88 (1H, d, J 8.0, Ph-H), 7.04 (3H, m, Ph-H), 7.19 (2H, d, J 9.0, Ph-H), 7.25 (1H, m, Ph-H), 7.31 (1H, br s, NH); MS 326.11 (M+H)⁺, C₁₄H₁₂ClNO₄S=325.77.

2,3-Dihydro-benzo[1,4]dioxine-6-sulfonic acid (3,5-bis-trifluoromethyl-phenyl)-amide 86. HPLC t_{R} 24.58 min (0-60%, 20 min); ¹H NMR (CDCl₃) δ: 4.21 (4H, m, CH₂), 6.86 (1H, d, J 8.5, Ph-H), 7.27 (1H, d, J 8.5, Ph-H), 7.30 (1H, d, J 2.0, Ph-H), 7.47 (2H, s, Ph-H), 7.50 (1H, s, Ph-H), 7.56 (1H, br-s, NH); MS 425.9 (M−2H)⁻, C₁₆H₁₁F₆NO₄S=427.32.

6-Phenoxy-pyridine-3-sulfonic acid (4-chloro-phenyl)-amide 87. HPLC t_{R} 23.20 min (10-70%, 20 min); ¹H NMR (CDCl₃) δ: 6.99 (1H, d, J 8.5, Ph-H), 7.11 (2H, d, J 8.5, Ph-H), 7.26 (1H, m, Ph-H), 7.41 (2H, m, Ph-H), 7.57 (3H, m, Ph-H), 7.63 (1H, s, Ph-H), 8.03 (1H, d, J 8.5, Ph-H), 8.57 (1H, br s, NH); MS 463.90 (M+2H)⁺, C₁₇H₁₃ClN₂O₃S=360.82.

5-Chloro-3-methyl-benzo[b]thiophene-2-sulfonic acid (4-chloro-3-nitro-phenyl)-amide 88. HPLC t_{R} 22.79 min (10-70%, 20 min); ¹H NMR (CDCl₃) δ: 2.55 (3H, s, CH₃), 7.38 (2H, m, Ph-H), 7.42 (1H, d, J 8.0, Ph-H), 7.66 (1H, s, Ph-H), 7.69 (1H, d, J 9.0, Ph-H), 7.73 (1H, s, Ph-H); MS 416.91 (M−H)⁻, C₁₅H₁₀Cl₂N₂O₄S₂=417.29.

N-(3,5-Bis-trifluoromethyl-phenyl)-4-pyrazol-1-yl-benzenesulfonamide 89. HPLC t_{R} 21.58 min (10-70%, 20 min); ¹H NMR (CD3OD) δ: 6.53 (1H, s, Ph-H), 7.57 (1H, s, Ph-H), 7.65 (2H, s, Ph-H), 7.73 (1H, s, Ph-H), 7.92 (4H, s, Ph-H), 8.28 (1H, s, Ph-H); MS 434.03 (M−H)⁻, C₁₇H₁₁F₆N₃O₂S=435.34

4-(4-Chloro-phenylsulfamoyl)-3,5-dimethyl-1H-pyrrole-2-carboxylic acid ethyl ester 90. HPLC t_{R} 20.99 min (0-60%, 20 min); ¹H NMR (CD₃OD) δ: 1.32 (3H, t, J 7.0, CH₂CH₃), 2.30 (3H, s, CH₃), 2.40 (3H, s, CH₃), 4.26 (2H, dd, J 7.0, 7.0, CH₂CH₃), 7.01 (2H, d, J 9.0, Ph-H), 7.20 (2H, d, J 9.0, Ph-H); MS 355.03 (M−H)⁻, C₁₅H₁₇ClN₂O₄S=356.83.

4-(3,5-Bis-trifluoromethyl-phenylsulfamoyl)-3,5-dimethyl-1H-pyrrole-2-carboxylic acid 91. HPLC t_{R} 23.28 min (0-60%, 20 min); ¹H NMR (CDCl₃) δ: 2.42 (3H, s, CH₃), 2.47 (3H, s, CH₃), 7.45 (3H, s, pH-H), 10.35 (1H, s, COOH); MS 428.98 (M−H)⁻, C₁₅H₁₂F₆N₂O₄S=430.32.

4-(4-Chloro-phenylsulfamoyl)-3,5-dimethyl-1H-pyrrole-2-carboxylic acid 92. HPLC t_{R} 19.59 min (0-60%, 20 min); ¹H NMR (CDCl₃) δ: 2.37 (3H, s, CH₃), 2.45 (3H, s, CH₃), 6.99 (2H, d, J 8.5, Ph-H), 7.07 (1H, br s, NH), 7.21 (2H, d, J 8.5, Ph-H); 9.47 (1H, br MS 327.26 (M−H)⁻, C₁₃H₁₃ClN₂O₄S=328.77.

2-(4-Chloro-phenylsulfamoyl)-4-methyl-thiazole-5-carboxylic acid ethyl ester 93. HPLC t_{R} 21.74 min (10-70%, 20 min); ¹H NMR (CDCl₃) δ: 1.26 (3H, t, J 7.5, CH₂CH₂₃), 2.47 (3H, s, CH₃), 4.21 (2H, dd, J 7.5, CH₂₃), 7.21 (2H, d, J 8.0, Ph-H), 7.28 (2d, J 8.0, Ph-H).

3,5-Dichloro-N-(4-chloro-phenyl)-4-hydroxy-benzenesulfonamide 94. HPLC t_{R} 21.65 min (10-60%, 20 min); ¹H NMR (CDCl₃) δ: 6.67 (1H, s, OH), 7.06 (2H, d, ArH, J 8.9), 7.21 (2H, d, ArH, J 8.9), 7.72 (2H, s, ArH), 8.30 (1H, s, NH); MS 352.01 (M−H)⁻, C₁₂H₈Cl₃NO₃S=352.62.

N-(3,5-Bis-trifluoromethyl-phenyl)-3,5-dichloro-4-hydroxy-benzenesulfonamide 95. HPLC t_{R} 24.06 min (10-60%, 20 min); ¹H NMR (CD₃OD) δ: 7.8 (2H, d, ArH, J 8.7), 7.68 (1H, s, ArH,), 7.67 (2H, s, ArH); MS 454.81 (M), C₁₄H₇Cl₂F₆NO₃S=454.17.

3,5-Dichloro-4-hydroxy-N-(4-trifluoromethyl-phenyl)-benzenesulfonamide 96. HPLC t_{R} 22.46 min (10-60%, 20 min); ¹H NMR (CD₃OD) δ: 7.28 (2H, d, ArH, J 8.7), 7.56 (2H, d, ArH, J 8.7), 7.67 (2H, s, ArH); MS 386.06 (M), C₁₃H₈Cl₂F₃NO₃S=386.17.

N-(4-Chloro-phenyl)-4-nitro-benzenesulfonamide 97. HPLC t_{R} 12.24 min (10-60%, 20 min); ¹H NMR (CD₃OD) δ: 7.08 (2H, d, ArH, J 8.8), 7.23 (2H, d, ArH, J 8.8), 7.96 (2H, d, ArH, J 8.8), 8.32 (2H, d, ArH, J 8.8). MS 311.33 (M−1)⁻, C₁₂H₉ClN₂O₄S=312.73.

N-(3,5-Bis-trifluoromethyl-phenyl)-4-nitro-benzenesulfonamide 98. HPLC t_{R} 15.36 min (10-60%, 20 min); ¹H NMR (CD₃OD) δ: 7.65 (1H, s, ArH,), 7.66 (2H, s, ArH), 8.06 (2H, d, ArH, J 9.0) 8.39 (2H, d, ArH, J 9.0); MS 413.43 (M−1)⁻, C₁₄H₈F₆N₂O₄S=414.28.

4-Amino-N-(3,5-bis-trifluoromethyl-phenyl)-3-chloro-benzenesulfonamide 99. HPLC t_{R} 18.38 min (10-60%, 20 min); ¹H NMR (CD₃OD) δ: 6.78 (1H, d, ArH, J 7.8), 7.41 (1H, d, ArH, J 7.8), 7.56 (1H, s, ArH), 7.62 (2H, s, ArH), 7.72 (1H, s, ArH); MS 419.77 (M+H)⁺, C₁₄H₉ClF₆N₂O₂S=418.74.

3-Nitro-N-(4-trifluoromethyl-phenyl)-benzenesulfonamide 100. HPLC t_{R} 23.04 min (10-60%, 20 min); ¹H NMR (DMSO-d6) δ: 7.30 (2H, d, ArH, J 8.3), 7.63 (2H, d, ArH, J 8.3), 8.06 (2H, d, ArH, J 8.8), 8.38 (2H, d, ArH, J 8.8), 11.17 (1H, s, NH); MS 345.05 (M−H)⁻, C₁₃H₉F₃N₂O₄S=346.28.

3,5-Dichloro-N-(3,5-dichloro-phenyl)-4-hydroxy-benzenesulfonamide 101. HPLC t_{R} 16.69 min (10-60%, 20 min); ¹H NMR (CD₃OD) δ: 7.08 (2H, s, ArH), 7.30 (1H, s, ArH), 7.71 (2H, s, ArH); MS 383.77 (M−H)⁻, C₁₂H₇Cl₄NO₃S=384.89.

4-Amino-3-chloro-N-(4-chloro-phenyl)-benzenesulfonamide 102. HPLC t_{R} 14.17 min (10-60%, 20 min); ¹H NMR (CD₃OD) δ: 6.75 (1H, d, ArH, J 8.3), 7.07, (2H, d, ArH, J 8.8), 7.28 (2H, d ArH, J 8.3), 7.32 (1H, d, ArH, J 8.8), 7.49 (1H, s, ArH); MS 317.43 (M), C₁₂H₁₀Cl₂N₂O₂S=317.19.

3-Chloro-N-(4-chloro-phenyl)-4-methoxy-benzenesulfonamide 103. HPLC t_{R} 16.65 min (10-60%, 20 min); ¹H NMR (DMSO-d₆) δ: 3.31 (3H, s, OCH₃), 7.08 (2H, d, ArH, J 8.3), 7.27 (1H, d, ArH, J 8.8), 7.30 (2H, d, ArH, J 8.3), 7.64 (1H, d, ArH, J 8.8), 7.74, (1H, s, ArH), 10.35, (1H, s, NH); MS 332.02 (M), C₁₃H₁₁Cl₂NO₃S=332.20.

N-(3,5-Bis-trifluoromethyl-phenyl)-3-chloro-4-methoxy-benzenesulfonamide 104. HPLC t_{R} 19.21 min (10-60%, 20 min); ¹H NMR (DMSO-d₆) δ: 3.30 (3H, s, OCH₃), 7.31 (1H, d, ArH, J 8.8), 7.65 (2H, s, ArH), 7.73 (1H, dd, ArH, J 8.8, 2.4), 7.77 (1H, s, ArH), 7.80 (1H, d, ArH, J 2.4), 11.12 (1H, s, NH); MS 433.98 (M), C₁₅H₁₀ClF₆NO₃S=433.75.

N-(3-Chloro-4-nitro-phenyl)-3,5-bis-trifluoromethyl-benzenesulfonamide 105. HPLC t_{R} 19.56 min (10-60%, 20 min); ¹H NMR (DMSO-d₆) δ: 7.38 (1H, dd, ArH, J 8.8, 2.4), 7.66 (1H, d, ArH, J 8.8), 7.76 (1H, d, ArH, J 2.4), 8.34 (2H, s, ArH), 8.51 (1H, s, ArH), 11.12 (1H, s, NH); MS 448.84 (M), C₁₄H₇ClF₆N₂O₄S=448.73.

3-(4-Acetyl-piperazin-1-yl)-N-(3,5-bis-trifluoromethyl-phenyl)-4-nitro-benzenesulfonamide 106. HPLC t_{R} 22.57 min (10-60%, 20 min); ¹H NMR (CD₃OD) δ: 2.15 (3H, s, CH₃), 3.20 (4H, m, CH₂), 3.68 (2H, m, CH₂), 3.84 (2H, m, CH₂), 7.12 (2H, m, ArH), 7.46 (1H, s, ArH), 8.26 (1H, s, ArH), 8.38 (2H, s, ArH); MS 540.09 (M), C₂₀H₁₈F₆N₄O₅S=540.44.

N-(3,5-Bis-trifluoromethyl-phenyl)-2-nitro-benzenesulfonamide 107. HPLC t_{R} 16.25 min (10-60%, 20 min); ¹H NMR (DMSO-d₆) δ: 7.58 (2H, s, ArH), 7.82 (1H, d, ArH, J 8.6), 7.88 (1H, dd, ArH, J 8.6, 8.6), 8.13 (1H, dd, ArH, J 8.6, 8.6), 8.37 (1H, s, ArH), 8.38 (1H, d, ArH, J 8.6), 11.60 (1H, s, NH); MS 414.85 (M), C₁₄H₈F₆N₂O₄S=414.28.

3-(3,5-Bis-trifluoromethyl-phenylsulfamoyl)-benzoic acid 108. HPLC t_{R} 22.39 min (10-60%, 20 min); ¹H NMR (CD₃OD) δ: 7.57 (2H, s, ArH), 7.62 (2H, s, ArH), 7.67 (1H, dd, ArH, J 7.8, 7.8), 7.97 (1H, dd, ArH, J 7.8, 1.0), 8.12 (1H, dd, ArH, J 7.8, 1.0), 8.30 (1H, d, ArH, J 1.0); MS 411.99 (M−2H)⁻, C₁₅H₉F₆NO₄S=413.29.

3,5-Dichloro-N-(4-chloro-benzyl)-4-hydroxy-benzenesulfonamide 109. HPLC t_{R} 24.04 min (10-60%, 20 min); ¹H NMR (CD₃OD) δ: 4.16 (2H, s, CH₂), 7.97 (2H, d, ArH), J 8.8), 8.81 (2H, s, ArH) 8.39 (2H, d, ArH, J 8.8); MS 365.26 (M−H)⁻, C₁₃H₁₀Cl₃NO₃S=366.65.

3,5-Dichloro-4-hydroxy-N-(4-trifluoromethyl-benzyl)-benzenesulfonamide 110. HPLC t_{R} 23.61 min (10-60%, 20 min); ¹H NMR (CD₃OD) δ: 4.24 (2H, s, CH₂), 7.32 (2H, d, ArH, J 8.7), 7.54 (2H, d, ArH, J 8.7), 7.81 (2H, s, ArH); MS 400.19 (M), C₁₄H₁₀Cl₂F₃NO₃S=400.20.

3,5-Dichloro-4-hydroxy-N-[2-(1H-indol-3-yl)-ethyl]-benzenesulfonamide 111. HPLC t_{R} 16.56 min (10-60%, 20 min); ¹H NMR (CD₃OD) δ: 2.95 (2H, d, CH₂, J 7.2), 3.32 (2H, s, CH₂, J 7.2), 6.99 (1H, s, ArH), 7.06 (1H, dd, ArH, J 8.1, 8.1), 7.17 (1H, dd, ArH, J 8.1, 8.1), 7.33 (1H, d, ArH, J 8.1), 7.39 (1H, d, ArH, J 8.1), 7.50 (2H, s, ArH); MS 382.82 (M−2H)⁻, C₁₆H₁₄Cl₂N₂O₃S=385.27.

4,5-Dibromo-thiophene-2-sulfonic acid (3,5-dichloro-phenyl)-amide 112. HPLC t_{R} 8.21 min (method B); MS 463.9 (M−H)⁻⁷⁹Br⁸¹Br³⁵Cl₂, C₁₀H₅Br₂Cl₂NO₂S₂=466.00.

N-(3,5-Dichloro-phenyl)-4-oxazol-2-yl-benzenesulfonamide 113. HPLC t_{R} 9.04 min (method B); MS 367.0 (M−H)⁻³⁵Cl₂, C₁₅H₁₀Cl₂N₂O₃S=369.22.

4-Bromo-5-chloro-thiophene-2-sulfonic acid (3,5-bis-trifluoromethyl-phenyl)-amide 114. HPLC t_{R} 9.22 min (method B); MS 487.9 (M−H)⁻⁸¹Br³⁵Cl, C₁₂H₅BrClF₆NO₂S₂=488.65.

4-Bromo-5-chloro-thiophene-2-sulfonic acid (3,5-dichloro-phenyl)-amide 115. HPLC t_{R} 9.12 min (method B); MS 419.9 (M−H)⁻⁸¹Br³⁵Cl₃, C₁₀H₅BrCl₃NO₂S₂=421.54.

5-Bromo-thiophene-2-sulfonic acid (4-trifluoromethyl-phenyl)-amide 116. HPLC t_{R} 8.28 min (method B); MS 385.9 (M−H)⁻⁸¹Br, C₁₁H₇BrF₃NO₂S₂=386.21.

5-Benzenesulfonyl-thiophene-2-sulfonic acid (3,5-bis-trifluoromethyl-phenyl)-amide 117. MS 514.2 (M−H)⁻, C₁₈H₁₁F₆NO₄S₃=515.47.

5-Benzenesulfonyl-thiophene-2-sulfonic acid (2,4-dichloro-phenyl)-amide 118. MS 446.0 (M−H)⁻³⁵Cl², C₁₆H₁₁Cl₂NO₄S₃=448.36.

5-Chloro-3-methyl-benzo[b]thiophene-2-sulfonic acid (3,5-bis-trifluoromethyl-phenyl)-amide 119. ¹H NMR (CDCl₃) δ: 2.51 (s, 3H, CH₃), 7.40 (dd, 1H, J 2.0, 8.7, Ar—H), 7.51 (s, 1H, NH), 7.55 (s, 3H, Ar—H), 7.67 (d, 1H, J 8.7, Ar—H), 7.69 (s, 1H, Ar—H); MS (accurate by FAB) 472.97412 (M)⁺³⁵Cl, C₁₇H₁₀ClF₆NO₂S₂=472.9746.

Benzo[b]thiophene-2-sulfonic acid (3,5-bis-trifluoromethyl-phenyl)-amide 120. ¹H NMR (CDCl₃) δ: 1H, NH), 7.45 (m, 5H, Ar—H), 7.83 (m, 1H, Ar—H), 8.06 (m, 1H, Ar—H), 8.24 (s, 1H, Ar—H); MS (accurate by FAB) 426.0049 (MH)⁺, C₁₆H₉F₆NO₂5₂=424.9979.

Benzo[1,2,5]thiadiazole-5-sulfonic acid (4-chloro-phenyl)-amide 121. ¹H NMR (CDCl₃) δ: 6.83 (s, 1H, NH), 7.00 (m, 2H, Ar—H), 7.16 (m, 2H, Ar—H), 7.79 (dd, 1H, J 1.8, 9.2, Ar—H), 8.03 (dd, 1H, J 0.7, 9.2, Ar—H), 8.46 (dd, J 0.7, 1.8, 1H, Ar—H); MS (accurate by FAB) 325.9821 (MH)⁺, C₁₂H₈ClN₃O₂S₂=324.9746.

Benzo[1,2,5]thiadiazole-5-sulfonic acid (3,5-bis-trifluoromethyl-phenyl)-amide 122. ¹H NMR (CDCl₃) δ: 7.43 (s, 1H, NH), 7.58 (s, 3H, Ar—H), 7.84 (dd, 1H, Ar—H), 8.09 (dd, 1H, J 1.8, 9.2, Ar—H), 8.03 (dd, 1H, J 0.7, 9.2, Ar—H), 8.57 (dd, J 0.7, 1.8, 1H, Ar—H); MS (accurate by FAB) 427.9960 (MH)⁺, C₁₄H₇F₆N₃O₂S₂=426.9884.

Benzo[1,2,5]thiadiazole-5-sulfonic acid (4-trifluoromethyl-phenyl)-amide 123. ¹H NMR (CDCl₃) δ: 7.19 (d, 2H, Ar—H), 7.44 (m, 3H, Ar—H & NH), 7.87 (dd, 1H, J 1.8, 9.2, Ar—H), 8.06 (dd, 1H, J 0.7, 9.2, AR-H), 8.58 (dd, J 0.7, 1.8, 1H, Ar—H); MS (accurate by FAB) 360.0080 (MH)⁺, C₁₃H₈F₃N₃O₂S₂=359.0010.

5-Pyridin-2-yl-thiophene-2-sulfonic acid (3,5-bis-trifluoromethyl-phenyl)-amide 124. ¹H NMR (CDCl₃) δ: 7.21 (m, 1H, pyridine-H), 7.40 (d, 1H, J 4.0, thiophene-H), 7.54 (s, 3H, Ph-H), 7.55 (d, 1H, J 4.0, thiophene-H), 7.61 (m, 1H, pyridine-H), 7.68 (m, 1H, pyridine-H), 7.80 (s, 1H, NH), 8.51 (m, 1H, pyridine-H); MS (accurate by FAB) 453.0164 (MH)⁺, C₁₇H₁₀F₆N₂O₂S₂=452.0088.

4,5-Dibromo-thiophene-2-sulfonic acid (4-chloro-phenyl)-amide 125. ¹H NMR (CDCl₃) δ: 6.91 (s, 1H, NH), 7.04 (d, 2H, J 6.7, Ph-H), 7.23 (s, 1H, thiophene-H), 7.24 (d, 2H, J 6.7, Ph-H); MS (accurate by FAB) 431.7963 (MH)⁺⁸¹Br⁷⁹Br³⁵Cl, C₁₀H₆Br₂ClNO₂S₂=428.7895.

4,5-Dibromo-thiophene-2-sulfonic acid (4-trifluoromethyl-phenyl)-amide 126. ¹H NMR (CDCl₃) δ: 7.20 (d, 2H, J 8.4, Ph-H), 7.33 (s, 1H, thiophene-H), 7.35 (s, 1H, NH), 7.53 (d, 2H, J 8.4, Ph-H); MS (accurate by FAB) 465.82122 (MH)⁺⁷⁹Br⁸¹Br, C₁₁H₆Br₂F₃NO₂S₂=465.1040.

### Example 2

General Method for the N-alkylation of Primary Bisarylsulfonamides

A primary bisarylsulfonamide from Example 1 (1.0 mol eq) was dissolved in anhydrous acetone and the solution was cooled to 0° C. before triethylamine (5.0 mol eq) was added slowly. After 30 min, alkylating agent (alkyl or aralkyl halide; 5.0 mol eq) was added slowly and the solution was stirred until TLC [heptane:ethyl acetate (2:1)] indicated the reaction to be complete. The reaction mixture was then concentrated, diluted with water, and extracted with ethyl acetate. After concentration of the extract, column chromatography of the residue [column (Isolute SI; Jones Chromatography), heptane:ethyl acetate (12:1→3:1)] afforded the desired alkylated sulfonamide.

The N-alkylated bisarylsulfonamide compounds of this invention were prepared in this manner. Analytical details for representative compounds are as follows:

5-Chloro-4-nitro-thiophene-2-sulfonic acid (4-fluoro-phenyl)-methyl-amide 7. TLC R_{F}=0.72 (heptane:ethyl acetate, 2:1); ¹H NMR (CDCl3) δ: 3.22 (3H, s, NCH₃), 7.02 (2H, m, Ph-H) 7.12 (2H, m, Ph-H), 7.73 (1H, d, J 1.0, thiophene-H).

5-Chloro-4-nitro-thiophene-2-sulfonic acid methyl-(4-trifluoromethyl-phenyl)-amide 50. ¹H NMR (CDCl₃) δ: 3.27 (3H, s, NMe), 7.30 (2H, d, J 8.5, Ph-H), 7.60 (2H, d, J 8.5, Ph-H), 7.75 (1H, s, thiophene).

5-Chloro-4-nitro-thiophene-2-sulfonic acid (4-chloro-phenyl)-methyl-amide 51. TLC R_{F}=0.69 (heptane:ethyl acetate, 2:1); HPLC t_{R} 18.49 min (20-80%, 20 min); ¹H NMR (CDCl₃) δ: 3.22 (3H, s, NCH₃), 7.08 (2H, d, J 8.5, Ph-H), 7.29 (2H, d, J 9.0, Ph-H), 7.38 (1H, s, thiophene-H).

5-Chloro-4-nitro-thiophene-2-sulfonic acid methyl-(4-trifluoromethyl-benzyl)-amide 52. TLC R_{F}=0.70 (heptane:ethyl acetate, 2:1); HPLC t_{R} 19.72 min (20-80%, 20 min); ¹H NMR (CDCl₃) δ: 2.71 (3H, s, NCH₃), 4.25 (2H, s, ArCH₂), 7.39 (2H, d, J 8.0, Ph-H), 7.58 (2H, d, J 8.0, Ph-H), 7.91 (1H, s, thiophene-H).

5-Chloro-4-nitro-thiophene-2-sulfonic acid benzyl-(4-fluoro-benzyl)-amide 53. ¹H NMR (CDCl₃) δ: 4.70 (2H, s, CH₂), 6.92 (2H, m, Ph-H), 6.97 (2H, m, Ph-H), 7.14 (2H, m, Ph-H), 7.19 (3H, m, Ph-H), 7.75 (1H, s, thiophene).

5-Chloro-4-nitro-thiophene-2-sulfonic acid benzyl-(4-chloro-phenyl)-amide 65. HPLC t_{R} 20.98 min (20-80%, 20 min); ¹H NMR (CDCl₃) δ: 4.78 (2H, s, CH₂), 7.00 (2H, m, Ph-H), 7.20 (2H, m, Ph-H), 7.26 (5H, m, Ph-H), 7.76 (1H, s, thiophene-H).

5-Chloro-4-nitro-thiophene-2-sulfonic acid benzyl-(4-methoxy-phenyl)-amide 66. HPLC t_{R} 22.76 min (0-60%, 20 min); ¹H NMR (CDCl₃) δ: 3.70 (3H, s, OMe), 4.69 (2H, s, CH₂), 6.72 (2H, d, J 8.5, Ph-H), 6.89 (2H, d, J 8.5, Ph-H), 7.19 (5H, m, Ph-H), 7.74 (1H, s, thiophene-H).

5-Chloro-4-nitro-thiophene-2-sulfonic acid (4-chloro-phenyl)-(3-trifluoromethyl-benzyl)-amide 67. HPLC t_{R} 23.62 min (0-60%, 20 min); ¹H NMR (CDCl₃) δ: 4.77 (2H, s, CH₂), 6.95 (2H, d, J 8.5, Ph-H), 7.19 (1H, s, Ph-H), 7.24 (2H, d, J 8.5, Ph-H), 7.37 (3H, m, Ph-H), 7.46 (1H, m, Ph-H), 7.79 (1H, s, thiophene-H).

5-Chloro-4-nitro-thiophene-2-sulfonic acid (3-trifluoromethyl-benzyl)-(4-trifluoromethylbenzyl)-amide 75. HPLC t_{R} 22.73 min (20-80%, 20 min); ¹H NMR (CDCl₃) δ: 4.38 (4H, s, CH₂), 7.19 (3H, m, Ph-H), 7.30 (2H, m, Ph-H), 7.45 (3H, m, Ph-H), 7.80 (1H, s, thiophene-H).

5-(1-Methyl-5-trifluoromethyl-1H-pyrazol-3-yl)-thiophene-2-sulfonic acid (3,5-bis-trifluoromethyl-phenyl)-(3-trifluoromethyl-benzyl)-amide 78. HPLC t_{R} 24.01 min (10-70%, 20 min); ¹H NMR (CDCl₃) δ: 4.03 (3H, s, NCH₃), 4.92 (2H, s, CH₂), 6.73 (1H, s, pyrazol-H), 7.23 (1H, dd, J 1.0, 4.0, Ph-H), 7.26 (1H, d, J 1.0, Ph-H), 7.46 (3H, m, Ph-H), 7.55 (2H, m, Ph-H), 7.59 (1H, d, J 7.5, Ph-H), 7.80 (1H, s, Ph-H); MS 682.08 (M+H)⁺, C₂₅H₁₅F₁₂N₃O₂S₂=681.52.

### Example 3

Fluorescence Polarisation Competitive Binding Assay

This was carried out using a 96-well microtiter plate (Costar) format. Recombinant HDM2 (1.5 μg per well) in TBS-BSA buffer (50 mM Tris pH 7.4, 150 mM NaCl, and 0.1% BSA) was incubated for 5 min at room temperature in the presence of serially diluted test compound (in TBS-BSA buffer with a final concentration of 5% DMSO). Fluorescently labelled p53-derived peptide (fluorescein-Met-Pro-Arg-Phe-Met-Asp-Tyr-Trp-Glu-Gly-Leu-Asn-NH₂, 0.2 μM) was added to each well and the plate was incubated at room temperature for 45 min. The fluorescence polarisation (excitation 485 nm, emission 520 nm) of the peptide was measured. IC₅₀ values were calculated from dose-response curves.

ELISA-type Competitive Binding Assay

Streptavidin-coated 96-well plates (Pierce Chemical Co., St. Louis, Mo., USA) were washed with TBS/BSA (25 mM Tris-HCl pH 7.5, 150 mM NaCl, 0.05% Tween 20, 0.1% BSA). Biotinylated p53-derived peptide (biotin-Ahx-Met-Pro-Arg-Phe-Met-Asp-Tyr-Trp-Glu-Gly-Leu-Asn-NH₂) was diluted to 1 μM with 25 mM Tris-HCl, pH 7.5, and aliquots (0.1 mL/well) were added to each well. After incubation for 1 h at room temperature with constant shaking, the plates were washed extensively with TBS/BSA buffer. HDM2 (50 μL/well of a 10 μg/mL stock solution in TBS/BSA buffer) and serially diluted (in TBS/BSA buffer) test compound (50 μL/well) were mixed and incubated for 30 min at room temperature. The reaction mixtures were then transferred to the peptide-coated streptavidin plates and incubation was continued for 1 h at room temperature, with constant shaking. The plates were then again washed extensively with TBS/BSA buffer. Anti-HDM2 antibody (SMP14 mAb, Santa Cruz Biotechnology, Inc., Santa Cruz, Calif., USA; 0.1 mL/well of a 0.125 μg/mL TBS/BSA buffer dilution) was added and incubated for 1 h at room temperature. After renewed washing, secondary antibody (anti-mouse Ab, Sigma Cat. No. A 4789; 0.1 mL/well of a 1:10,000 TBS/BSA buffer dilution) was added and incubated for 1 h at room temperature. After extensive washing, TMB ELISA reagent (Pierce; 0.1 mL/well) was added, incubated for 1 min, and the colour reaction stopped by addition of 2 M aq H₂SO₄ (0.1 mL/well). The absorbance at 450 nm was then measured. IC₅₀ values were calculated from dose-response curves.

### Example 4

Primary screening data of representative bisarylsulfonamide compounds are summarised in Table 1. The fluorescence polarisation competitive HDM2 binding assay described in Example 3 was used. Furthermore, the anti-proliferative potency of the compounds against three human tumour cell lines with different genetic make-up was also determined. The anti-proliferative IC₅₀ values stated in Table 1 were determined using a standard 72-h MTT cytotoxicity assay [Haselsberger K, Peterson D C, Thomas D G, Darling J L; Anti Cancer Drugs 1996; 7: 331-338]. The cell lines used were as follows: AGS, gastric adenocarcinoma, wild-type p53; H1299, large cell lung carcinoma, p53 null, low HDM2; SJSA-1, primitive multipotential bone sarcoma, wild-type p53, overexpressed HDM2.

Compounds active in the fluorescence polarisation competitive HDM2 binding assay were subsequently also tested in the corresponding ELISA-style assay format (Example 3). Similar IC₅₀ values were obtained from both assay formats. In general very good correlation between in vitro competitive HDM2-binding and cellular anti-proliferative activities was observed for bisarylsulfonamides. Thus only compounds capable of antagonising p53 peptide binding of HDM2 in vitro possessed anti-proliferative activity. Furthermore, the potency ranking of the compounds in the in vitro and cellular assays was very similar. These results suggest strongly that the anti-proliferative effects observed are due to the bisarylsulfonamides modulating cellular HDM2 function. This conclusion is supported by the fact that in general the cell line in which HDM2 is overexpressed (SJSA-1) was less sensitive to active bisarylsulfonamide test compounds than the other two cell lines assayed. The fact that anti-proliferative effects can be achieved in tumour cells devoid of functional p53 through modulation of HDM2 is indicated by the fact that the p53^{-/-} cell line H1299 was similarly responsive to the test compounds as the other two cell lines with normal p53.

### Example 5

Representative bisarylsulfonamide compounds were tested on a panel of transformed and non-transformed human cell lines, using a standard 72-h MTT cytotoxicity assay [Haselsberger K et al, ibid]. The results are summarised in Table 2.

It can be seen that compounds 2, 3, 6, & 7 were 3- to 4-fold more potent in their anti-proliferative activity against transformed compared to non-transformed cell lines. Furthermore, potency for these compounds on average was higher against carcinoma compared to sarcoma cell lines, in contrast to the control compound roscovitine, which exerts its anti-proliferative effects through a different mechanism, viz. inhibition of cell-cycle cyclin-dependent kinases [Wang S, McClue S J, Ferguson J R, Hull J D, Stokes S, Parsons S, Westwood R, F ischer P M; Tetrahedron: Asymmetry 2001; 12: 2891-2894]. This differential selectivity further supports the notion that the bisarylsulfonamides modulate cellular HDM2 function. It is known that HDM2 amplification is particularly common in soft tissue sarcomas and osteosarcomas [Momand J et al, ibid; Bartel F, Meye A, Wurl P, Kappler M, Bache M, Lautenschlager C, Grunbaum U, Schmidt H, Taubert H; Int. J. Cancer 2001; 95: 168-175].

### Example 6

p53 Reporter Gene Assay

A dual assay assessing both cell viability and p53 response was employed. A U2OS-derived cell line stably transfected with a p53 response element and a luciferase reporter gene was created. The genetic expression cassette construct GC3p53tkaLuc used for this purpose has been described [Zhu J, Gao B, Zhao J, Balmain A; Cancer Gene Ther. 2000; 7: 4-12]. After seeding of cells into 96-well tissue culture plates, treatment with test compounds, and incubation for the desired duration, the cell proliferation reagent WST-1 (Roche Molecular Biochemicals) was used to determine cell viability. The Bright-Glo™ Luciferase Assay System (Promega) was used to measure luciferase activity. The data from the cell viability assay was used to normalise data from the luciferase assay. Assay protocols were as previously described [Krausz E, Watt K, Cummings L, Baxter C, Blake D G; Biochemica 2001: 26-27].

E2F Reporter Gene Assay

A similar system to the p53 reporter assay described above was used, with the exception that it was designed to be responsive to E2F transcription factors rather than p53. An A549-derived cell line stably transfected with an E2F response element and a luciferase reporter gene was created. The genetic expression cassette construct used was from Clontech (Mercury™ Cell Cycle Profiling System). Assays were carried out as described above.

### Example 7

Representative bisarylsulfonamides were tested in the p53 gene reporter assay described in Example 6. The results are summarised in Table 3.

It has previously been reported that a p53-derived optimised cell-permeable peptide (the positive control peptide in the table above) was capable of activating the p53 pathway in tumour cells through inhibiting the HDM2/p53 interaction [Chène P, Fuchs J, Bohn J, Garcia-Echeverria C, Furet P, Fabbro D; J. Molec. Biol. 2000; 299: 245-253]. Our results confirm this finding (see above). Furthermore, representative bisarylsulfonamides also caused dramatic induction of p53-responsive luciferase activity in the same assay system. In general it was found that compounds which were inactive in the in vitro competitive HDM2-binding assay did not exhibit this ability to induce cellular p53. For example, the effect of the marginally active compound 4 was much less pronounced than that of the more active compounds. The fact that the bisarylsulfonamides were able to activate cellular p53 transactivation activity confirms that they modulate cellular HDM2 activity.

### Example 8

Representative bisarylsulfonamides were tested in the E2F gene reporter assay described in Example 6. The results are summarised in Table 4.

Only bisarylsulfonamides active as HDM2 antagonists were capable of down-regulation of E2F transcriptional activity in the gene reporter assay. This effect was observed regardless of whether an asynchronous cell population or cells synchronised at the late G1/S phase [Ji C, Marnett L J, Pietenpol J A; Oncogene 1997; 15: 2749-2753] were used. The fact that bisarylsulfonamides were able to suppress E2F transcriptional activity confirms that they modulate the cellular HDM2/E2F interaction.

### Example 9

It is well established that radiotherapy and most current forms of chemotherapy cause extensive genetic damage and induction of the p53-dependent apoptotic pathway, which determines the important role of this tumor suppressor protein in cancer therapy. HDM2, on the other hand, as a transcriptional product and negative regulator pf p53, would destabilise p53 and diminish its pro-apoptotic function. Inhibitors of the p53-HDM2 negative feedback loop (e.g. compounds disrupting the p53-HDM2 complex) are expected to synergise with cytotoxic agents for induction of p53 and subsequent apoptosis. To investigate this hypothesis we have used as a model system three cell lines: AGS (gastric adenocarcinoma, wild type p53), H1299 (colon adenocarcinoma, p53 null) and SJSA-1 (osteosarcoma, wild type p53, over-expressed HDM2) and two cytotoxic agents, cisplatin (DNA alkylating agent) and etoposide (topoisomerase inhibitor), well known to induce p53 and apoptosis. As a representative bisarylsulfonamide inhibitor of the HDM2-p53 interaction, compound 2 was used at the anti-proliferative IC₅₀ concentration determined for each cell line, i.e. 1, 5, and 15 μM, respectively. The combination treatment regimen included addition of 2 to the cells at the same time, 6 hours before, or 6 hours after cisplatin or etoposide. Both chemotherapeutic agents were used in the concentration range of 100-0.19 μM. The cytotoxic effect of etoposide and cisplatin in the presence and absence of the HDM2-p53 inhibitor was determined using a standard MTT assay after a total of 72 hours incubation time. The cytotoxicity results for cisplatin are shown in Table 5.

Compound 2 increased the cytotoxic effect of cisplatin on all three cell lines used. The synergistic effect was highest when 2 was added prior to the treatment with cisplatin and the potency increase in AGS, H1299 and SJSA-1 cells was 18.5-, 16- and 357-fold, respectively.

Similar synergistic effects of 2 was observed when the compound was used in combination with etoposide. The results are shown in Table 6.

The cytotoxicity of etoposide was significantly increased in the presence of 2, as the highest effect was registered when the inhibitor of the HDM2-p53 interaction was added 6 hours before etoposide. The IC₅₀ values calculated for etoposide in AGS, H1299 and SJSA-1 cells pre-treated with 2 were 3.5-, 25- and 726-fold lower than the ones not receiving 2.

The synergistic effect of bisarylsulfonamides and the cytotoxic agents was highest in SJSA-1 cells. This result was expected as SJSA-1 cells over-express HDM2, suggesting that DNA damage-induced p53 will be destabilised and the effect of the cytotoxic agents diminished. The relative resistance of SJSA-1 towards treatment with etoposide and cisplatin may be due to the anti-apoptotic effect of HDM2. Disrupting the interaction between HDM2 and p53 allows quick accumulation and stabilisation of p53 and induction of cell death. The chemosensitisation effects of bisarylsulfonamides involves a p53-independent mechanism as well sinse it was observed in H1299 cells, which are p53 null.

### Example 10

Mode of Action Studies

Many types of cellular stress converge on the p53 pathway and induce its activity. It was therefore important to demonstrate that the compounds of this invention activate p53 directly by targeting the interaction between p53 and HDM2 and not indirectly by targeting the activation of upstream kinases, for example. The biological effect of bisarylsufonamides on cellular level was studies using representative compounds (mainly compound 2) and number of cell lines with different HDM2 and p53 status. These were overexpressing Hdm2 (SJSA1—osteosarcoma), wild type p53 and normal levels of HDM2 (AGS—gastric adenocarcinoma, MCF7—breast adenocarcinoma), or p53 null, low levels of HDM2 (H1299—large cell lung carcinoma). The effects of bisarylsulfonamides were analysed at the cellular levels of key proteins in the HDM2 pathway and were compared with those induced by well-characterised anti-cancer drugs such as etoposide and cisplantinum.

AGS, H1299, MCF7 or SJSA1 cells were treated with five times the anti-proliferative IC₅₀ concentration of compound 2 or in some cases etoposide or cisplatinum (FIG. 1). Cells were collected at intervals and morphological changes were noted at the time of collection. Cells were lysed and equal amounts were analysed by SDS-polyacrylamide gel electrophoresis (SDS-PAGE) and immunoblotting. Treatment of AGS or MCF7 cells with etoposide or cisplatinum resulted in classical stress response (FIG. 1B, 1C, and 1D), inducing p53 phosphorylation of serine 15, as well as increasing the p53 and E2F-1 protein levels. At later time points this correlated with PARP cleavage; production of an 85-kDa cleavage product is a marker for apoptosis that coincided with morphological changes characteristic of cell death. In contrast, treatment of AGS, H₂₁₉₉, MCF7 or SJSA1 cells with compound 2 induced a rapid reduction in HDM2 and E2F-1 levels (but not E2F-2) with no induction of p53 nor p53 phosphorylation at serine 15 (FIG. 1A, 1B, 1D and 1E). The reduction in E2F-1 levels correlated with reduction in E2F transcriptional activity as determined by an E2F-dependent luciferase reporter assay (refer Example 6). Treatment of MCF7 cells with 4-bromo-5-chloro-thiophene-2-sulfonic acid 4-trifluoromethyl-benzylamide (negative control compound), a compound, which does not inhibit the HDM2—p53 interaction in vitro, but has similar structure to compound 2, did not induce any notable changes in the protein levels tested or cell morphology (FIG. 1D).

Compound 2 induced rapid morphological changes that were most dramatic in SJSA1, cells that have a very prominent cytoplasmic component. Rapid cell rounding was detected in all cell types tested.

Analysis of other stress pathways in MCF7 cells after compound 2 treatment showed a transient induction in JNK phosphorylation but no clear induction of the p38 MAPK pathway. Anisomycin (mRNA translation inhibitor) was used as a positive control for the induction of these stress pathways (FIG. 1D). The induction of JNK phosphorylation was not confirmed in the other cell types.

Induction of PARP cleavage was very rapid in H1299 cells but less so in AGS, MCF7 or SJSA1 cells (FIG. 1A, 1B, 1C, 1D and 1E). In the MCF7 cells despite the absence of the 85-kDa PARP cleavage product there was a dramatic reduction in PARP protein levels (FIG. 1D). The effect of compound 49, another in vitro inhibitor, was also tested on AGS, SJSA1 and H1299 (FIG. 2). This induced even more rapid cell rounding as well as similar changes in protein levels seen with compound 2.

It was important to determine whether the rapid cell rounding corresponded to cell death. MCF7 or H1299 cells were treated with compound 2 and the number of rounded cells as well as the number of viable cells were counted at intervals, using trypan blue exclusion. Rapid cell rounding occurred with very different kinetics to cell death in both cell types (FIG. 3). This data suggests that cell rounding does not correlate with dead cells.

A pair of compounds, 68 (competitive inhibition IC₅₀=431 μM) and 69 (IC₅₀=151 μM; refer Table 1) were analysed for their effects on MCF7 and H1299 cells. Cells were treated at five times the IC₅₀ concentration of the more active compound 69. Little change in protein levels were detected when cells were treated with 68. In contrast, 69 induced changes in both morphology and protein levels. Rapid reduction in PARP levels was detected in both cell lines with the production of the 85-kDa cleavage product detectable at later time points (FIG. 4A and 4B). HDM2 and HDMX levels decreased in MCF7 cells shortly after treatment with compound 69 (FIG. 4A). In H1299 cells compound 69 treatment induced rapid decrease of HDM2 levels, which recovered at a later time point (FIG. 4B). E2F-1 levels barely changed compared to controls (FIG. 4B). No rapid cell rounding was detected in either cell type. By 68 hours floating MCF7 cells (with morphology corresponding to the one of dead cells) were detected with compound 69 treatment but not with compound 68 treatment. This was distinct from the cell rounding seen with compound 2.

In order to determine whether bisarylsulfonamides induced cell cycle changes, MCF7 or H1299 cells were treated with cisplatin or compound 2 (FIG. 5). Both cisplatin and compound 2 induced sub-G1 cells, indicative of apoptosis, with no detectable arrest at any phase of the cell cycle.

To characterise the apoptosis-inducing properties of bisarylsulfonamides caspase ⅓ activty was measured. AGS or H1299 cells were treated with a low concentration of compound 2 or cisplatin corresponding to twice the IC₅₀ value (FIG. 6A). Morphological changes were detected in 10-20% of cells by 72 hours corresponding to an induction of caspase activity first detected at 48 hours, increasing by 72 hours. We also treated AGS and H1299 cells with higher concentrations corresponding to five times the IC₅₀ (FIG. 6B). In this experiment we included a specific caspase 3 inhibitor, Z-VAD.fmk to determine the caspase responsible. Caspase activity was detected by four hours in H1299 correlating with the PARP cleavage product detected by Western blotting (FIG. 1C). In AGS cells, only weak caspase activity was induced by 24 hours compared with cisplatin treatment. This activity was caspase 3-dependent because the caspase inhibitor reduced the caspase activity to background levels at 24 hours (FIG. 6B). Notably, rapid cell rounding was detected in AGS cells despite the presence of the caspase inhibitor, further confirming our results that cell rounding was not an indication of cell death.

Our results consistently show that bisarylsulfonamides, inhibitors of HDM2, can induce a rapid decrease in the levels of E2F-1. To determine if this was an off-target effect or one that could occur by inhibition of HDM2/HDMX activities, two different siRNAs against HDM2 and one agains HDMX were used to transfect MCF7 cells. First, the cells were transfected with HDM2 or control siRNAs serially every 24 hours and collected 24 hours later. Cells were lysed and equal amounts of protein were separated by SDS-PAGE and analysed by Western blotting. Treatment with control siRNAs had no effect on the levels of HDM2 (FIG. 7A lanes 1-9). Treatment with HDM2 siRNA markedly reduced the levels of HDM2 protein at all time points (FIG. 7A, lanes 10-15). This corresponded to a decrease in E2F-1 levels but not E2F-4 levels and an induction of p53 protein levels. Actin was used as a loading control. HDMX has been shown to stabilise HDM2 levels. Next, we determined whether HDM2 levels were affected by HDMX levels in MCF7 cells and whether reducing HDMX levels could reduce E2F-1 levels. MCF7 cells were transfected with HDM2 siRNA and HDMX siRNA alone or together. HDM2 levels were reduced at all time points in the presence of HDM2 siRNA (FIG. 7B lanes 10-12). Treatment of cells with HDMX siRNA reduced cellular levels of HDMX as well as reducing the levels of HDM2 (FIG. 7B lanes 13-15). In both cases. E2F-1 levels were also decreased. Combined treatment with HDM2 and HDMX siRNA had an additive effect greatly reducing the levels of E2F-1 at 48 hours. (FIG. 7B lane 17). These experiments show that both HDM2 and HDMX can regulate E2F-1. It is likely that HDMX regulates E2F-1 indirectly by stabilising HDM2.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

TABLE 1

| | | | IC₅₀ (μM) | | | |
|---|---|---|---|---|---|---|
| | | | In vitro | | | |
| | | | competitive | | | |
| | | | HDM2 | | | |
| | | | binding | Anti-proliferative effect on cell line | | |
| No. | Structure | Name | assay | AGS | H 1299 | SJSA-4 |

| | | | | | | |
|---|---|---|---|---|---|---|
| 1 | 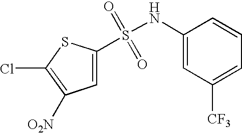 | 5-Chloro-4-nitro- thiophene-2-sulfonic acid (3-trifluoro- methyl-phenyl)-amide | 409 ± 84 | n.d. | n.d. | n.d. |
| 2 | 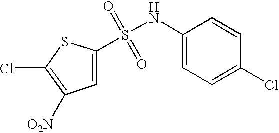 | 5-Chloro-4-nitro- thiophene-2-sulfonic acid (4-chloro-phenyl)- amide | 26.4 ± 3.4 | 1.1 ± 0.2 | 5.1 ± 0.7 | 13.6 ± 1.1 |
| 3 | 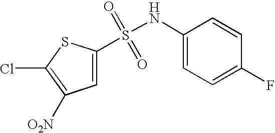 | 5-Chloro-4-nitro- thiophene-2-sulfonic acid (4-fluoro-phenyl)- amide | 41.9 ± 5.8 | 1.5 ± 0.3 | 5.7 ± 0.4 | 17.1 ± 0.4 |
| 4 | 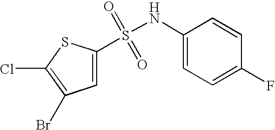 | 4-Bromo-5-chloro- thiophene-2-sulfonic acid (4-fluoro-phenyl)- amide | 378 | n.d. | n.d. | n.d. |
| 5 | 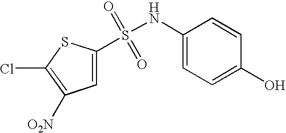 | 5-Chloro-4-nitro- thiophene-2-sulfonic acid (4-hydroxy- phenyl)-amide | 105 ± 26 | 2.2 ± 0.6 | 8.7 ± 2.2 | 24.0 ± 2.1 |
| 6 | 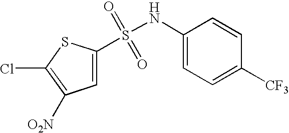 | 5-Chloro-4-nitro- thiophene-2-sulfonic acid (4-trifluoro- methyl-phenyl)-amide | 20.4 ± 1.4 | 2.0 ± 0.3 | 8.1 ± 1.3 | 22.5 ± 0.6 |
| 7 | 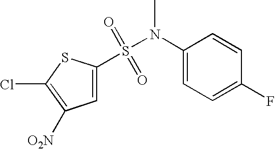 | 5-Chloro-4-nitro- thiophene-2-sulfonic acid (4-fluoro-phenyl)- methyl-amide | 15.4 ± 7.8 | 7.0 ± 1.3 | 12.9 ± 0.7 | 9.3 ± 2.7 |
| 8 | 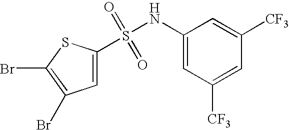 | 4,5-Dibromo- thiophene-2-sulfonic acid (3,5-bis- trifluoromethyl- phenyl)-amide | 407 | n.d. | n.d. | n.d. |
| 9 | 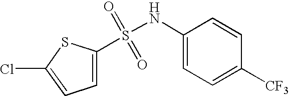 | 5-Chloro-thiophene-2- sulfonic acid (4- trifluoromethyl- phenyl)-amide | 102 ± 13 | n.d. | n.d. | n.d. |
| 10 | 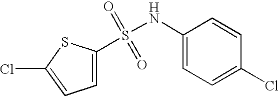 | 5-Chloro-thiophene-2- sulfonic acid (4- -chloro-phenyl)-amide | 320 | n.d. | n.d. | n.d. |
| 11 | 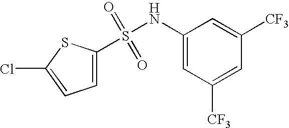 | 5-Chloro-thiophene-2- sulfonic acid (3,5-bis- trifluoromethyl- phenyl)-amide | 435 ± 76 | n.d. | n.d. | n.d. |
| 12 | 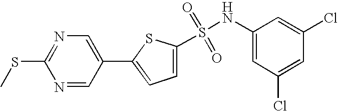 | 5-(2-Methylsulfanyl- pyrimidin-5-yl)- thiophene-2-sulfonic acid (3,5-dichloro- phenyl)-amide | 374 ± 12 | n.d. | n.d. | n.d. |
| 13 | 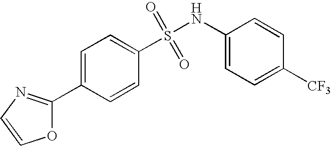 | 4-Oxazol-2-yl-N-(4- trifluoromethyl- phenyl)-benzene- sulfonamide | 494 | n.d. | n.d. | n.d. |
| 14 | 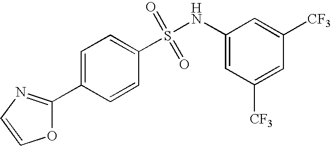 | N-(3,5-Bis- tifluoromethyl- phenyl)-4-oxazol-2-yl- benzenesulfonamide | 448 ± 55 | n.d. | n.d. | n.d. |
| 15 | 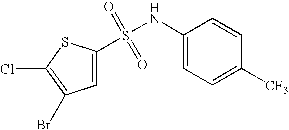 | 4-Bromo-S-chloro- thiophene-2-sulfonic acid (4-trifluoro- methyl-phenyl)-amide | 218 ± 15 | n.d. | n.d. | n.d. |
| 16 | 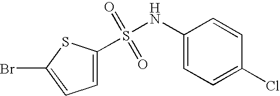 | 5-Bromo-thiophene-2- sulfonic acid (4- chloro-phenyl)-amide | 290 ± 54 | n.d. | n.d. | n.d. |
| 17 | 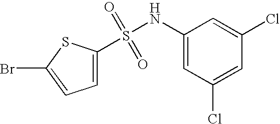 | 5-Bromo-thiophene-2- sulfonic acid (3,5- dichloro-phenyl)- amide | 291 | n.d. | n.d. | n.d. |
| 18 | 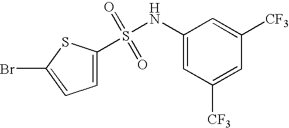 | 5-Bromo-thiophene-2- sulfonic acid (3,5-bis- trifluoromethyl- phenyl)-amide | 172 ± 21 | n.d. | n.d. | n.d. |
| 19 | 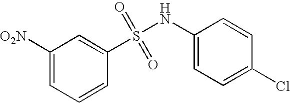 | N-(4-Chloro-phenyl)- 3-nitro-beuzene- sulfonamide | 295 ± 45 | n.d. | n.d. | n.d. |
| 20 | 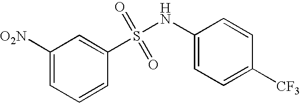 | 3-Nitro-N-(4- trifluoromethyl- phenyl)-benzene- sulfonamide | 482 ± 26 | n.d. | n.d. | n.d. |
| 21 | 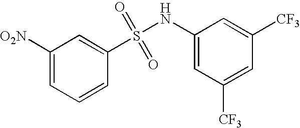 | N-(3,5-Bis- trifluoromethyl- phenyl)-3-nitro- benzenesulfonamide | 209 ± 38 | n.d. | n.d. | n.d. |
| 22 | 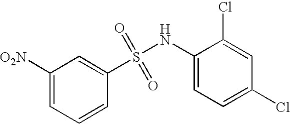 | N-(2,4-Dichloro- phenyl)-3-nitro- benzenesulfonamide | 441 ± 69 | n.d. | n.d. | n.d. |
| 23 | 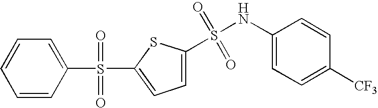 | 5-Benzenesulfonyl- thiophene-2-sulfonic acid (4-trifluoro- methyl-phenyl)-amide | 146 ± 5 | n.d. | n.d. | n.d. |
| 24 | 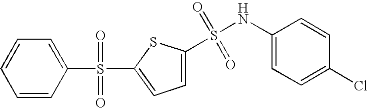 | 5-Benzenesulfonyl- thiophene-2-sulfonic acid (4-chloro-phenyl)- amide | 243 ± 59 | n.d. | n.d. | n.d. |
| 25 | 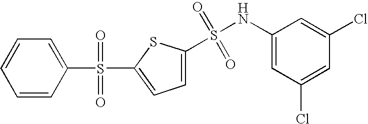 | 5-Benzenesulfonyl- thiophene-2-sulfonic acid (3,5-dichloro- phenyl)-amide | 288 ± 39 | n.d. | n.d. | n.d. |
| 26 | 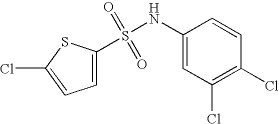 | 5-Chloro-thiophene-2- sulfonic acid (3,4- dichloro-phenyl)- amide | 295 ± 36 | n.d. | n.d. | n.d. |
| 27 | 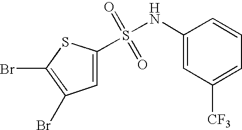 | 4,5-Dibromo- thiophene-2-sulfonic acid (3-trifluoro- methyl-phenyl)-amide | 199 ± 28 | n.d. | n.d. | n.d. |
| 28 | 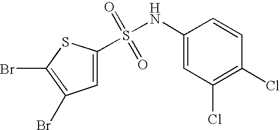 | 4,5-Dibromo- thiophene-2-sulfonic acid (3,4-dichloro- phenyl)-amide | 160 ± 31 | n.d. | n.d. | n.d. |
| 29 | 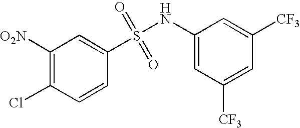 | N-(3,5-Bis- trifluoromethyl- phenyl)-4-chloro-3- nitro-benzene- sulfonamide | 129 ± 22 | n.d. | n.d. | n.d. |
| 30 | 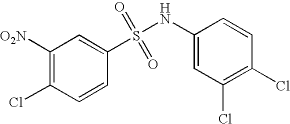 | 4-Chloro-N-(3,4- dichloro-phenyl)-3- nitro-benzene- sulfonamide | 427 ± 3 | n.d. | n.d. | n.d. |
| 31 | 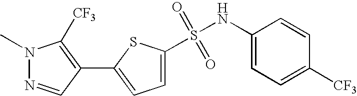 | 5-(1-Methyl-5- trifluoromethyl-1H- pyrazol-4-yl)- thiophene-2-sulfonic acid (4-trifluoro- methyl-phenyl)-amide | 350 ± 10 | n.d. | n.d. | n.d. |
| 32 | 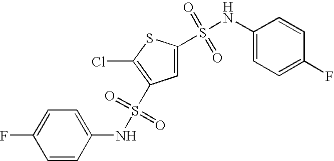 | 5-Chloro-thiophene- 2,4-disulfonic acid bis- [(4-fluoro-phenyl)- amide] | 408 ± 31 | n.d. | n.d. | n.d. |
| 33 | 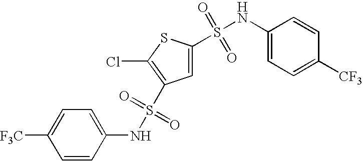 | 5-Chloro-thiophene- 2,4-disulfonic acid bis- [(4-trifluoro-methyl- phenyl)-amide] | 65 ± 5 | 68 ± 1 | 69 ± 1 | >100 |
| 34 | 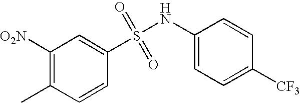 | 4-Methyl-3-nitro-N-(4- trifluoromethyl- phenyl)-benzene- sulfonamide | 295 ± 23 | n.d. | n.d. | n.d. |
| 35 | 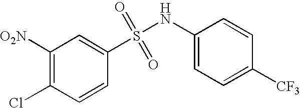 | 4-Chloro-3-nitro-N-(4- trifluoromethyl- phenyl)-benzene- sulfonamide | 192 ± 8 | 13.0 ± 3.3 | 9.3 ± 1.7 | 26.5 ± 1.6 |
| 36 | 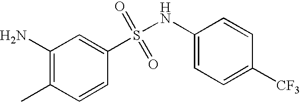 | 3-Amino-4-methyl-N- (4-trifluoromethyl- phenyl)-benzene- sulfonamide | 429 | n.d. | n.d. | n.d. |
| 37 | 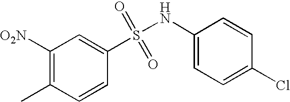 | N-(4-Chloro-phenyl)- 4-methyl-3-nitro- benzene-sulfonamide | 294 ± 52 | n.d. | n.d. | n.d. |
| 38 | 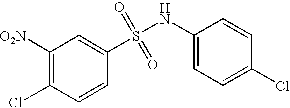 | 4-Chloro-N-(4-chloro- phenyl)-3-nitro- benzene-sulfonamide | 232 ± 13 | 16 ± 4 | 11 ± 4 | 22 ± 4 |
| 39 | 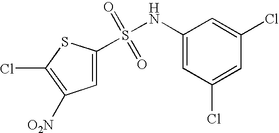 | 5-Chloro-4-nitro- thiophene-2-sulfonic acid (3,5-dichloro- phenyl)-amide | 46.1 ± 3.4 | 4.9 ± 1.6 | 12.1 ± 4.0 | 27.2 ± 0.4 |
| 40 | 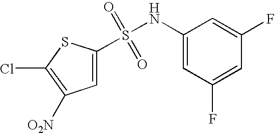 | 5-Chloro-4-nitro- thiophene-2-sulfonic acid (3,5-difluoro- phenyl)-amide | 59.6 ± 10.5 | 4.0 ± 0.3 | 10.6 ± 0.4 | 26.4 ± 0.2 |
| 41 | 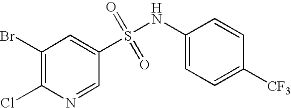 | 5-Bromo-6-chloro- pyridine-3-sulfonic acid(4- trifluoromethyl- phenyl)-amide | 258 ± 12 | 31 ± 2 | 28 ± 0 | 53 ± 3 |
| 42 | 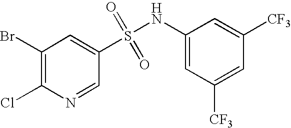 | 5-Bromo-6-chloro- pyridine-3-sulfonic acid(3,5-bis- trifluoromethyl- phenyl)-amide | 278 ± 50 | 30 ± 7 | 32 ± 7 | 65 ± 18 |
| 43 | 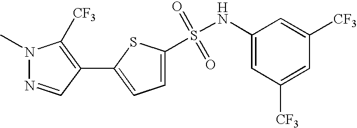 | 5-(1-Methyl-5- trifluoro-methyl-1H- pyrazol-4-yl)- thiophene-2-sulfonic acid (3,5-bis-trifluoro- methyl-phenyl)-amide | 133 ± 11 | 20.9 ± 1.0 | 18.0 ± 0.8 | 33.7 ± 0.3 |
| 44 | 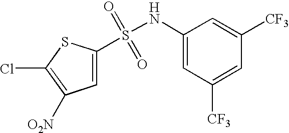 | 5-Chloro-4-nitro- thiophene-2-sulfonic acid (3,5-bis- trifluoromethyl- phenyl)-amide | 25.4 ± 0.2 | 10 ± 0 | 14 ± 2 | 19 ± 3 |
| 45 | 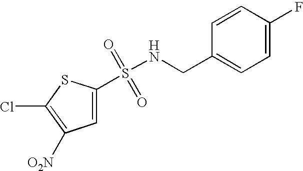 | 5-Chloro-4-nitro- thiophene-2-sulfonic acid 4-fluoro- benzylamide | 16.0 ± 3.7 | 7.5 ± 4.4 | 11.1 ± 3.1 | 11.5 ± 3.0 |
| 46 | 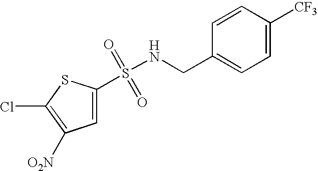 | 5-Chloro-4-nitro- thiophene-2-sulfonic acid 4-trifluoromethyl- benzylamide | 11.1 ± 1.7 | 1.3 ± 0.5 | 3.4 ± 0.5 | 4.4 ± 1.1 |
| 47 | 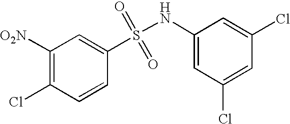 | 4-Chloro-N-(3,5- dichloro-phenyl)-3- nitro-benzene- sulfonamide | 234 ± 37 | 23 ± 2 | 18 ± 8 | 37 ± 6 |
| 48 | 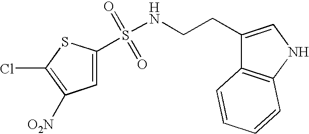 | 5-Chloro-4-nitro- thiophene-2-sulfonic acid [2-(1H-indol-3- yl)-ethyl]-amide | 25.3 ± 4.6 | 2.6 ± 1.4 | 3.6 ± 0.9 | 3.1 ± 1.0 |
| 49 | 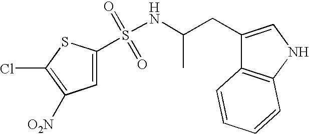 | 5-Chloro-4-nitro- thiophene-2-sulfonic acid [2-(1H-indol-3- yl)-1-methyl-ethyl]- amide | 46.3 ± 4.0 | 2.2 ± 2.3 | 1.5 ± 0.4 | 2.0 ± 0.2 |
| 50 | 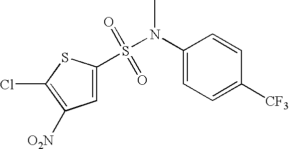 | 5-Chloro-4-nitro- acid methyl-(4- thiophene-2-sulfonic acid methyl-(4- trifluoromethyl- phenyl)-amide | 7.3 ± 0.6 | 30.3 ± 0.3 | 34.7 ± 6.1 | 35.4 ± 11.5 |
| 51 | 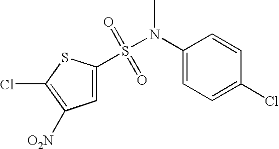 | 5-Chloro-4-nitro- thiophene-2-sulfonic acid (4-chloro-phenyl)- methyl-amide | 5.9 ± 0.7 | 11.3 ± 1.0 | 17.4 ± 3.8 | 15.6 |
| 52 | 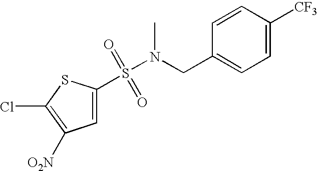 | 5-Chloro-4-nitro- thiophene-2-sulfonic acid methyl-(4- trifluoromethyl- benzyl)-amide | 10.7 ± 0.2 | 23.3 ± 1.4 | 28.5 ± 1.9 | 27.5 ± 1.3 |
| 53 | 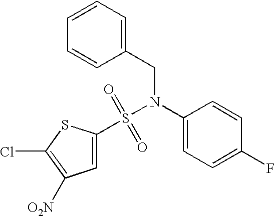 | 5-Chloro-4-nitro- thiophene-2-sulfonic acid benzyl-(4-fluoro- benzyl)-amide | 15 | 3.6 ± 0.3 | 5.6 ± 1.0 | 5.1 ± 0.4 |
| 54 | 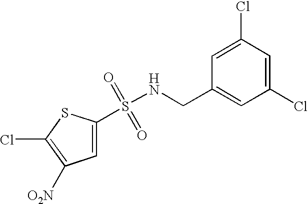 | 5-Chloro-4-nitro- thiophene-2-sulfonic acid 3,5-dichloro- benzylamide | 20.2 ± 1.3 | 5.6 ± 0.2 | 9.7 ± 2.9 | 12.0 ± 0.4 |
| 55 | 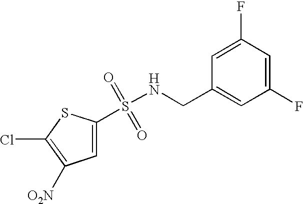 | 5-Chloro-4-nitro- thiophene-2-sulfonic acid 3,5-difluoro- benzylamide | 27.0 ± 10.1 | 7.0 ± 0.0 | 9.3 ± 2.5 | 13.3 ± 2.1 |
| 56 | 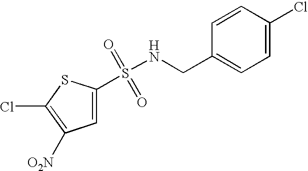 | 5-Chloro-4-nitro- thiophene-2-sulfonic acid 4-chloro- benzylamide | 24.8 ± 5.4 | 4.3 ± 0.6 | 6.3 ± 1.9 | 9.3 ± 2.7 |
| 57 | 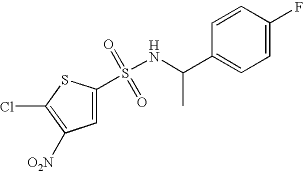 | 5-Chloro-4-nitro- thiophene-2-sulfonic acid [1-(4-fluoro- phenyl)-ethyl]-amide | 31.1 ± 5.6 | 1.7 ± 0.3 | 2.7 ± 0.6 | 5.9 ± 0.2 |
| 58 | 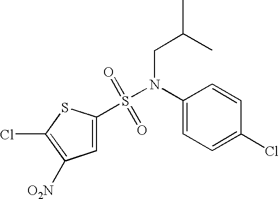 | 5-Chloro-4-nitro- thiopbene-2-sulfonic acid (4-chloro-phenyl)- isobutyl-amide | 249 ± 30 | n.d. | n.d. | n.d. |
| 59 | 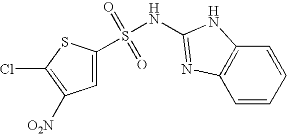 | 5-Chloro-4-nitro- thiophene-2-sulfonic acid (1H-benzo- imidazol-2-yl)-amide | 40 ± 1 | n.d. | n.d. | n.d. |
| 60 | 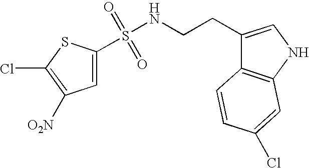 | 5-Chloro-4-nitro- thiophene-2-sulfonic acid [2-(6-chloro-1H- indol-3-yl)-ethyl]- amide | 29 ± 6 | n.d. | n.d. | n.d. |
| 61 | 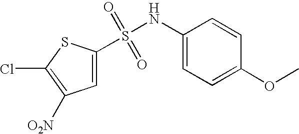 | 5-Chloro-4-nitro- thiophene-2-sulfonic acid (4-methoxy- phenyl)-amide | 45 ± 14 | 1.8 ± 0.4 | 2.7 ± 0.4 | 2.8 ± 0.2 |
| 62 | 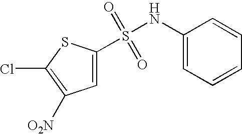 | 5-Chloro-4-nitro- thiophene-2-su1fonic acid phenylamide | 67 ± 1 | n.d. | n.d. | n.d. |
| 63 | 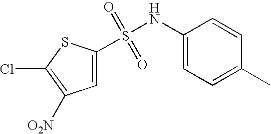 | 5-Chloro-4-nitro- thiophene-2-sulfonic acid p-tolylamide | 35 ± 8 | 1.7 ± 0.6 | 3.9 ± 0.9 | 5.7 ± 1.6 |
| 64 | 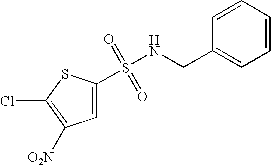 | 5-Chloro-4-nitro- thiophene-2-sulfonic acid benzylamide | 18 ± 0 | 3.6 ± 0.2 | 6.3 ± 2.1 | 7.3 ± 1.7 |
| 65 | 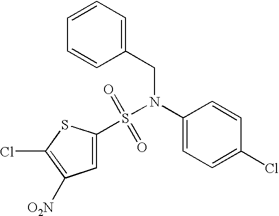 | 5-Chloro-4-nitro- thiophene-2-sulfonic acid benzyl-(4-chloro- phenyl)-amide | 7.4 ± 1.0 | 5.2 ± 2.2 | 10.8 ± 4.3 | 11.1 ± 6.7 |
| 66 | 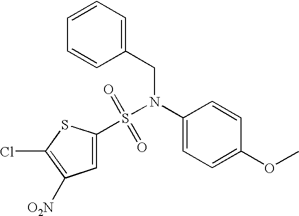 | 5-Chloro-4-nitro- thiophene-2-sulfonic acid benzyl-(4- methoxy-phenyl)- amide | 14.7 ± 0.9 | 2.4 ± 0.2 | 3.9 ± 0.5 | 3.9 ± 1.3 |
| 67 | 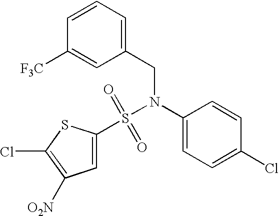 | 5-Chloro-4-nitro- thiophene-2-sulfonic acid (4-chloro-phenyl)- (3-trifluoromethyl- benzyl)-amide | 8.5 ± 3.2 | 6.7 ± 0.9 | 12.0 ± 1.0 | 11.8 ± 1.6 |
| 68 | 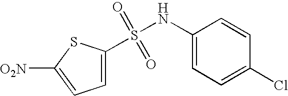 | 5-Nitro-thiophene-2- sulfonic acid (4- chloro-phenyl)-amide | 431 ± 37 | 76 | n.d. | n.d. |
| 69 | 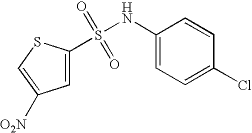 | 4-Nitro-thiophene-2- sulfonic acid (4- chloro-phenyl)-amide | 151 ± 24 | 19 | 30 | 28 |
| 70 | 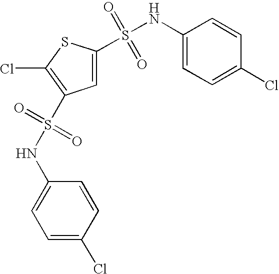 | 5-Chloro-thiophene- 2,4-disulfonic acid bis- [(4-chloro-phenyl)- amide] | 135 ± 19 | n.d. | n.d. | n.d. |
| 71 | 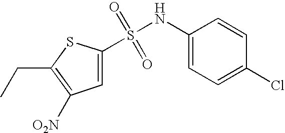 | 5-Ethyl-4-nitro- thiophene-2-sulfonic acid (4-chloro-phenyl)- amide | 237 ± 35 | n.d. | n.d. | n.d. |
| 72 | 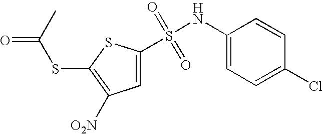 | Thioacetic acid S-[5- (4-chloro- phenylsulfamoyl)-3- nitro-thiophen-2-yl]ester | 16.2 ± 0.0 | 61 | n.d. | n.d. |
| 73 | 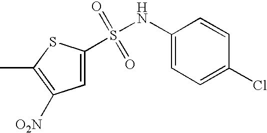 | 5-Methyl-4-nitro- thiophene-2-sulfonic acid (4-chloro-phenyl)- amide | >167 | 53 | 66 | n.d. |
| 74 | 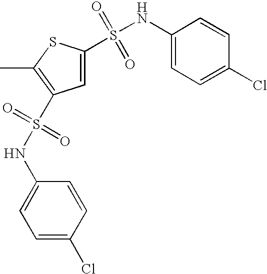 | 5-Methyl-thiophene- 2,4-disulfonic acid bis- [(4-chloro-phenyl)- amide] | 123 ± 15 | n.d. | n.d. | n.d. |
| 75 | 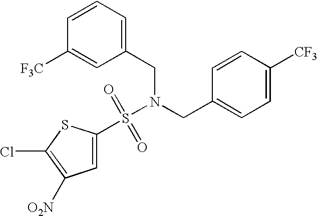 | 5-Chloro-4-nitro- thiophene-2-sulfonic acid (3-trifluoro- methyl-benzyl)-(4- trifluoromethyl- benzyl)-amide | 3.5 ± 10 | 9 | 16 | 18 |
| 76 | 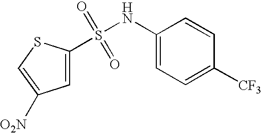 | 4-Nitro-thiophene-2- sulfonic acid (4- trifluoromethyl- phenyl)-amide | 366 ± 7 | n.d. | n.d. | n.d. |
| 77 | 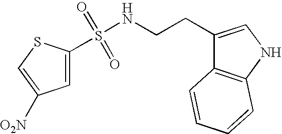 | 4-Nitro-thiophene-2- sulfonic acid [2-(1H- indol-3-yl)-ethyl]- amide | 247 ± 61 | n.d. | n.d. | n.d. |
| 78 | 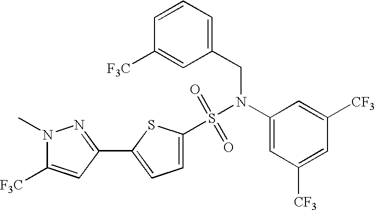 | 5-(1-Methyl-5-tri- fluoromethyl-1H- pyrazol-3-yl)- thiophene-2-sulfonic acid (3,5-bis-tri- fluoromethyl phenyl)-(3-trifluoro- methyl-benzyl)-amide | 315 ± 83 | n.d. | n.d. | n.d. |
| 79 | 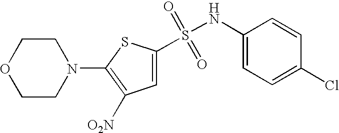 | 5-Morpholin-4-yl-4- nitro-thiophene-2- sulfonic acid (4- chloro-phenyl)-amide | 419 ± 31 | n.d. | n.d. | n.d. |
| 80 | 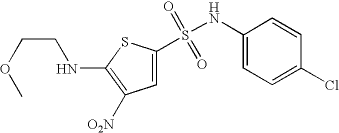 | 5-(2-Methoxy- ethylamino)-4-nitro- thiophene-2-sulfonic acid (4-chloro-phenyl)- amide | 154 ± 27 | n.d. | n.d. | n.d. |
| 81 | 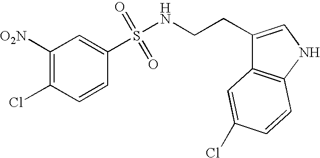 | 4-Chloro-N-[2-(5- chloro-1H-indol-3-yl)- ethyl]-3-nitro- benzenesulfonamide | 311 ± 21 | 29 ± 2 | 39 ± 6 | 51 ± 1 |
| 82 | 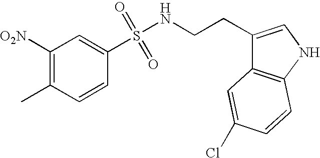 | N-[2-(5-Chloro-1H- indol-3-yl)-ethyl]-4- methyl-3-nitro- benzenesulfonamide | 338 ± 111 | 50 ± 0 | 49 ± 0 | 65 ± 4 |
| 83 | 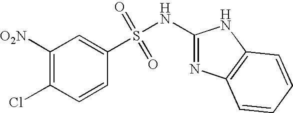 | N-(1H-Benzoimidazol 2-yl)-4-chloro-3-nitro- benzenesulfonamide | 119 ± 43 | 0.9 ± 0.1 | 1.4 ± 0.2 | 2.3 ± 0.9 |
| 84 | 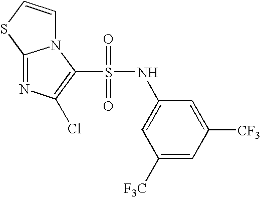 | 6-Chloro-imidazo[2,1- b]thiazole-5-sulfonic acid (3,5-bis-trifluoro- methyl-phenyl)-amide | 308 ± 17 | n.d. | n.d. | n.d. |
| 85 | 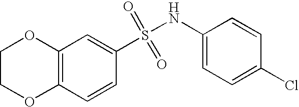 | 2,3-Dihydro- benzo[1,4]dioxine-6- sulfonic acid (4- chloro-phenyl)-amide | 432 ± 7 | n.d. | n.d. | n.d. |
| 86 | 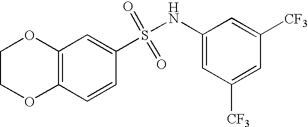 | 2,3-Dihydro- benzo[1,4]dioxine-6- sulfonic acid (3,5-bis- trifluoromethyl- phenyl)-amide | 375 ± 39 | n.d. | n.d. | n.d. |
| 87 | 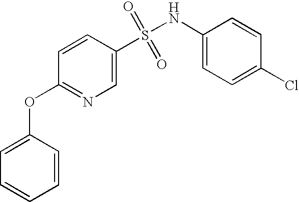 | 6-Phenoxy-pyridine-3- sulfonic acid (4- chloro-phenyl)-amide | 212 ± 27 | n.d. | n.d. | n.d. |
| 88 | 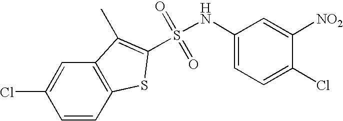 | 5-Chloro-3-methyl- benzo[b]thiophene-2- sulfonic acid (4- chloro-3-nitro-phenyl)- amide | 174 ± 11 | n.d. | n.d. | n.d. |
| 89 | 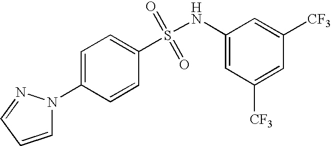 | N-(3,5-Bis- trifluoromethyl- phenyl)-4-pyrazol-1- yl-benzenesulfonamide | 275 ± 45 | n.d. | n.d. | n.d. |
| 90 | 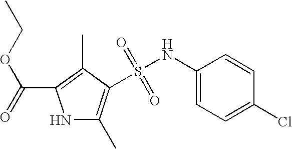 | 4-(4-Chloro- phenylsulfamoyl)-3,5- dimethyl-1H-pyrrole- 2-carboxylic acid ethyl ester | 331 ± 52 | n.d. | n.d. | n.d. |
| 91 | 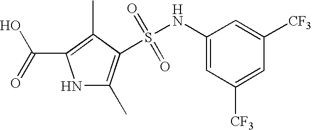 | 4-(3,5-Bis- trifluoromethyl- phenylsulfamoyl)-3,5- dimethyl-1H-pyrrole- 2-carboxylic acid | 481 | n.d. | n.d. | n.d. |
| 92 | 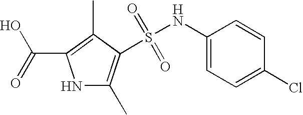 | 4-(4-Chloro- phenylsulfamoyl)-3,5- dimethyl-1H-pyrrole- 2-carboxylic acid | 418 | n.d. | n.d. | n.d. |
| 93 | 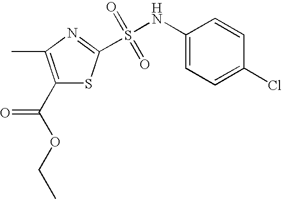 | 2-(4-Chloro- phenylsulfamoyl)-4- methyl-thiazole-5- carboxylic acid ethyl ester | 373 ± 373 | n.d. | n.d. | n.d. |
| 94 | 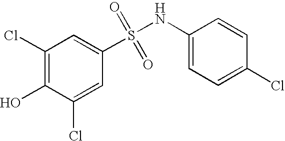 | 3,5-Dichloro-N-(4- chloro-phenyl)-4- hydroxy- benzenesulfonamide | 227 ± 7 | n.d. | n.d. | n.d. |
| 95 | 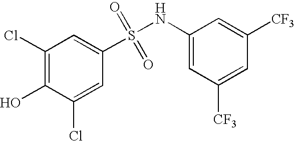 | N-(3,5-Bis- trifluoromethyl- phenyl)-3,5-dichloro- 4-hydroxy- benzenesulfonamide | 243 ± 11 | n.d. | n.d. | n.d. |
| 96 | 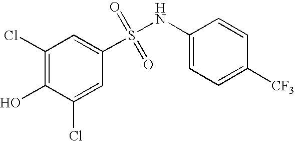 | 3,5-Dichloro-4- hydroxy-N-(4- trifiluoromethyl- phenyl)- benzenesulfonamide | 449 ± 51 | n.d. | n.d. | n.d. |
| 97 | 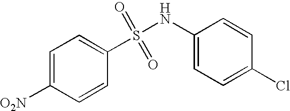 | N-(4-Chloro-phenyl)- 4-nitro-benzene- sulfonamide | 394 ± 13 | n.d. | n.d. | n.d. |
| 98 | 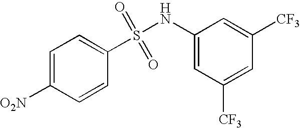 | N-(3,5-Bis- trifluoromethyl- phenyl)-4-nitro- benzenesulfonamide | 207 ± 0 | n.d. | n.d. | n.d. |
| 99 | 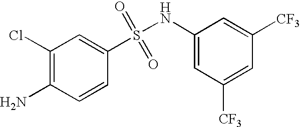 | 4-Amino-N-(3,5-bis- trifluoromethyl phenyl)-3-chloro- benzenesulfonamide | 248 ± 29 | n.d. | n.d. | n.d. |
| 100 | 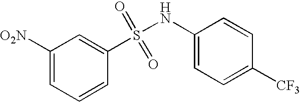 | 3-Nitro-N-(4- trifluoromethyl- phenyl)- benzenesulfonamide | 431 ± 5 | n.d. | n.d. | n.d. |
| 101 | 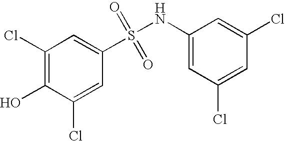 | 3,5-Dichloro-N-(3,5- dichloro-phenyl)-4- hydroxy- benzenesulfonamide | 299 ± 31 | n.d. | n.d. | n.d. |
| 102 | 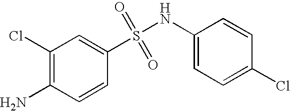 | 4-Amino-3-chloro-N- (4-chloro-phenyl)- benzenesulfonamide | 467 ± 35 | n.d. | n.d. | n.d. |
| 103 | 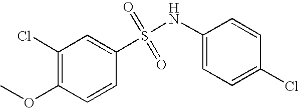 | 3-Chloro-N-(4-chloro- phenyl)-4-methoxy- benzenesulfonamide | 408 ± 18 | n.d. | n.d. | n.d. |
| 104 | 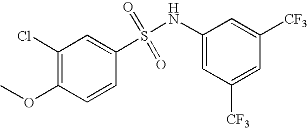 | N-(3,5-Bis- trifluoromethyl- phenyl)-3-chloro-4- methoxy-benzene- sulfonamide | 151 ± 26 | 25 | n.d. | n.d. |
| 105 | 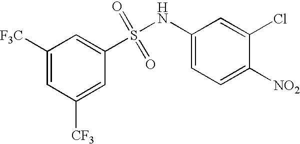 | N-(3-Chloro-4-nitro- phenyl)-3,5-bis- trifluoromethyl- benzenesulfonamide | 153 ± 18 | 24 ± 3 | 25 ± 1 | 37 ± 18 |
| 106 | 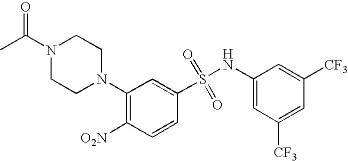 | 3-(4-Acetyl-piperazin- 1-yl)-N-(3,5-bis- trifluoromethyl- phenyl)-4-nitro- benzenesulfonamide | 241 ± 158 | n.d. | n.d. | n.d. |
| 107 | 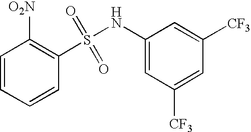 | N-(3,5-Bis- trifluoromethyl- phenyl)-2-nitro- benzenesulfonamide | 401 ± 46 | n.d. | n.d. | n.d. |
| 108 | 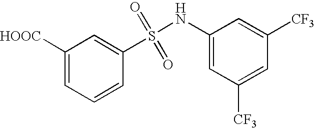 | 3-(3,5-Bis- trifluoromethyl- phenylsulfamoyl)- benzoic acid | 451 ± 118 | n.d. | n.d. | n.d. |
| 109 | 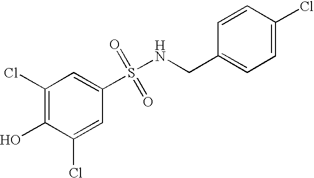 | 3,5-Dichloro-N-(4- chloro-benzyl)-4- hydroxy- benzenesulfonamide | 375 ± 41 | n.d. | n.d. | n.d. |
| 110 | 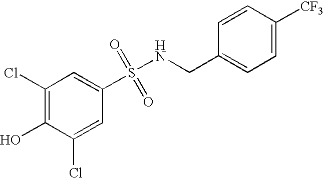 | 3,5-Dichloro-4- hydroxy-N-(4- trifluoromethyl- benzyl)- benzenesulfonamide | 242 | n.d. | n.d. | n.d. |
| 111 | 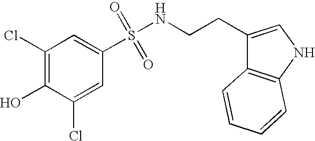 | 3,5-Dichloro-4- hydroxy-N-[2-(1H- indol-3-yl)-ethyl]- benzenesulfonamide | 448 | n.d. | n.d. | n.d. |
| 112 | 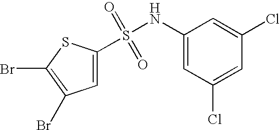 | 4,5-Dibromo- thiophene-2-sulfonic acid (3,5-dichloro- phenyl)-amide | 194 | n.d. | n.d. | n.d. |
| 113 | 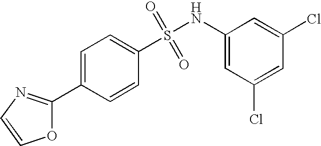 | N-(3,5-Dichloro- phenyl)-4-oxazol-2-yl- benzenesulfonamide | 246 | n.d. | n.d. | n.d. |
| 114 | 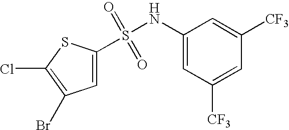 | 4-Bromo-5-chloro- thiophene-2-sulfonic acid (3,5-bis- trifluoromethyl- phenyl)-amide | 293 | n.d. | n.d. | n.d. |
| 115 | 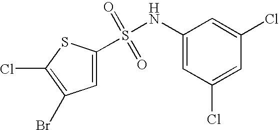 | 4-Bromo-5-chloro- thiophene-2-sulfonic acid (3,5-dichloro- phenyl)-amide | 431 | n.d. | n.d. | n.d. |
| 116 | 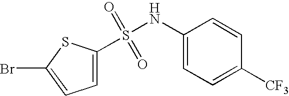 | 5-Bromo-thiophene-2- sulfonic acid (4- trifluoromethyl- phenyl)-amide | 152 | n.d. | n.d. | n.d. |
| 117 | 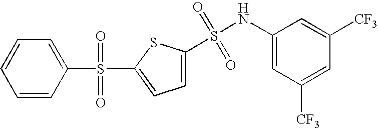 | 5-Benzenesulfonyl- thiophene-2-sulfonic acid (3,5-bis- trifluoromethyl- phenyl)-amide | 147 | n.d. | n.d. | n.d. |
| 118 | 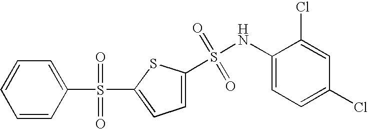 | 5-Benzenesulfonyl- thiophene-2-sulfonic acid (2,4-dichloro- phenyl)-amide | 305 | n.d. | n.d. | n.d. |
| 119 | 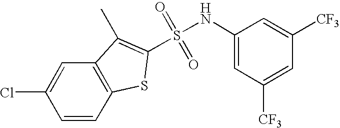 | 5-Chloro-3-methyl- benzo[b]thiophene-2- sulfonic acid (3,5-bis- trifluoromethyl- phenyl)-amide | 106 ± 13 | n.d. | n.d. | n.d. |
| 120 | 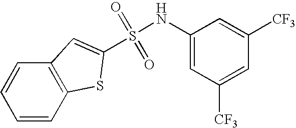 | Benzo[b]thiophene-2- sulfonic acid (3,5-bis- trifluoromethyl- phenyl)-amide | 193 ± 12 | n.d. | n.d. | n.d. |
| 121 | 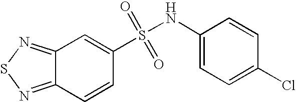 | Benzo[1,2,5]thiadiazol e-5-sulfonic acid (4- chloro-phenyl)-amide | 314 ± 29 | n.d. | n.d. | n.d. |
| 122 | 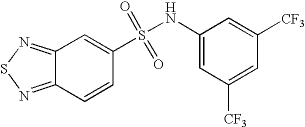 | Benzo[1,2,5]thiadiazol e-5-sulfonic acid (3,5- bis-trifluoromethyl- phenyl)-amide | 165 ± 8 | n.d. | n.d. | n.d. |
| 123 | 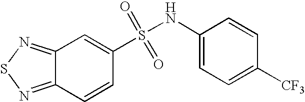 | Benzo[1,2,5]thiadiazol e-5-sulfonic acid (4- trifluoromethyl- phenyl)-amide | 329 ± 42 | n.d. | n.d. | n.d. |
| 124 | 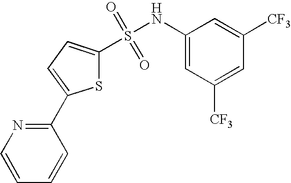 | 5-Pyridin-2-yl- thiophene-2-sulfonic acid (3,5-bis- trifluoromethyl- phenyl)-amide | 265 ± 64 | n.d. | n.d. | n.d. |
| 125 | 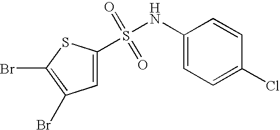 | 4,5-Dibromo- thiophene-2-sulfonic acid (4-chloro-phenyl)- amide | 198 ± 17 | n.d. | n.d. | n.d. |
| 126 | 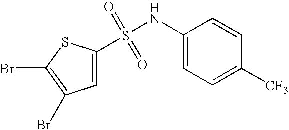 | 4,5-Dibromo- thiophene-2-sulfonic acid (4- trifluoromethyl- phenyl)-amide | 163 ± 27 | n.d. | n.d. | n.d. |
| 127 | 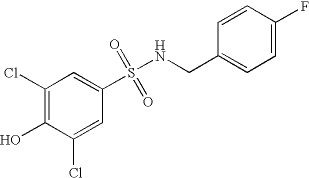 | 3,5-Dichloro-N-(4- fluoro-benzyl)-4- hydroxy- benzenesulfonamide | 325 ± 24 | n.d. | n.d. | n.d. |
| 128 | 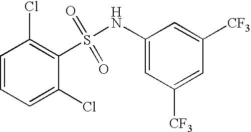 | N-(3,5-Bis- trifluoromethyl- phenyl)-2,6-dichloro- benzenesulfonamide | 134 ± 28 | 53 | n.d. | n.d. |
| 129 | 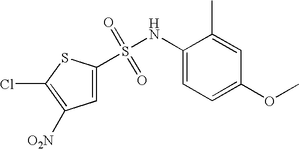 | 5-Chloro-4-nitro- thiophene-2-sulfonic acid (4-methoxy-2- methyl-phenyl)-amide | 278 ± 19 | n.d. | n.d. | n.d. |
| 130 | 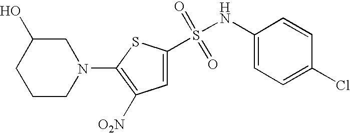 | 5-(3-Hydroxy- piperidin-1-yl)-4-nitro thiophene-2-sulfonic acid (4-chloro-phenyl)- amide | 64 ± 1 | n.d. | n.d. | n.d. |
| 131 | 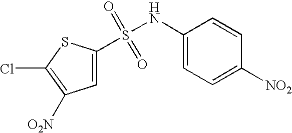 | 5-Chloro-4-nitro- thiophene-2-sulfonic acid (4-nitro-phenyl)- amide | 41 ± 4 | n.d. | n.d. | n.d. |

n.d. denoted not determined.

TABLE 2

| | | Anti-proliferative activity, 72-h MTT IC₅₀ (μM) | | | | |
|---|---|---|---|---|---|---|
| Cell line | | 2 | 3 | 6 | 7 | Roscovitine |
| AGS | Gastric adenocarcinoma | 1.2 | 1.7 | 2.2 | 5.0 | 10.7 |
| DU145 | Prostate carcinoma | 3.1 | 3.6 | 6.1 | 9.1 | 8.5 |
| HT29 | Colon adenocarcinoma | 2.1 | 2.3 | 3.5 | 4.6 | 15.6 |
| Lovo | Colon adenocarcinoma | 2.1 | 2.3 | 3.9 | 8.5 | 13.8 |
| Nci-H460 | Large cell lung carcinoma | 3.3 | 5.2 | 8.0 | 9.6 | 12.8 |
| SK-N-MC | Neuriepithelioma | 3.0 | 3.8 | 2.9 | 3.0 | 7.1 |
| A549 | Lung carcinoma | 5.2 | 5.9 | 10.0 | 19.3 | 9.2 |
| H1299 | Large cell lung carcinoma | 4.4 | 5.2 | 4.9 | 14.0 | 11.9 |
| HCT116 | Colon carcinoma | 2.8 | 3.5 | 6.0 | 12.2 | 9.7 |
| HeLa | Cervical carcinoma | 2.8 | 2.7 | 3.9 | 13.7 | 16.1 |
| MCF7 | Breast adenocarcinoma | 2.7 | 1.9 | 3.3 | 11.2 | 12.1 |
| Messa | Uterus sarcoma | 13.1 | 18.3 | 16.0 | 8.1 | 9.9 |
| Messa-Dx5 | Uterus sarcoma | 9.5 | 12.1 | 15.5 | 7.6 | 5.3 |
| Saos-2 | Osteosracoma | 9.5 | 8.9 | 12.0 | 13.3 | 17.4 |
| NaCat | Human keratinocytes | 9.3 | 10.5 | 10.6 | 12.9 | 16.3 |
| SJSA-1 | Osteosracoma | 13.5 | 14.1 | 17.8 | 17.0 | 17.8 |
| U2OS | Osteosracoma | 6.2 | 8.2 | 10.2 | 8.8 | 16.4 |
| SKUT-1 | Uterus leiomyosarcoma | 8.8 | 14.1 | 15.0 | 6.1 | 10.5 |
| HS27 | Foreskin fibroblasts | 17.3 | 15.2 | 21.4 | 39.2 | 43.1 |
| IMR90 | Lung fibroblasts | 23.1 | 19.8 | 29.8 | 41.8 | 44.2 |
| WI38 | Lung fibroblasts | 22.7 | 21.1 | 30.0 | 52.3 | 34.5 |

| | | | | | |
|---|---|---|---|---|---|
| Average transformed | 5.7 ± 3.9 | 6.9 ± 5.0 | 8.4 ± 5.1 | 10.2 ± 4.4 | 12.3 ± 3.7 |
| Average carcinomas | 3.1 ± 1.2 | 3.8 ± 1.8 | 4.9 ± 2.3 | 9.7 ± 4.7 | 11.2 ± 3.0 |
| Average sarcomas | 10.2 ± 2.7 | 12.1 ± 3.8 | 13.7 ± 3.1 | 11.3 ± 3.7 | 13.8 ± 5.1 |
| Average non-transformed | 21.0 ± 3.1 | 18.7 ± 3.1 | 27.1 ± 4.9 | 44.6 ± 6.9 | 40.6 ± 5.3 |

TABLE 3

| | | Fold | |
|---|---|---|---|
| | | induction of | In vitro competitive |
| | Concentration^{b} | luciferase | HDM2 binding |
| Compound | (μM) | activity^{c} | assay^{d} IC₅₀ (μM) |
| Control^{a} | 150-200 | 4 | 0.19 |
| 2 | 6 | 231 | 26.4 ± 3.4 |
| 3 | 6 | 200 | 41.9 ± 5.8 |
| 4 | 100^{e} | <2 | 378 |
| 6 | 5.5 | 84 | 20.4 ± 1.4 |
| 7 | 9 | 32 | 15.4 ± 7.8 |
| 39 | 15 | 64 | 46.1 ± 3.4 |
| 40 | 13 | 68 | 59.6 ± 10.9 |
| 45 | 15 | 67 | 16 ± 3.7 |
| 46 | 6 | 55 | 11.1 ± 1.7 |
| 48 | 5.5 | 43.5 | 25.3 ± 4.6 |
| 49 | 5.5 | 117 | 46.3 ± 4 |
| 50 | 39 | 21 | 7.3 ± 0.6 |
| 54 | 10 | 90 | 20.2 ± 1.3 |
| 55 | 10 | 53 | 27 ± 10.1 |
| 57 | 7 | 25 | 31.1 ± 5.6 |
| 60 | 7.5 | 48 | 29 ± 6 |
| 61 | 4.8 | 34 | 45 ± 14 |
| 63 | 6.4 | 95 | 35 ± 8 |
| 129 | 100 | 5 | 278 ± 19 |
| 65 | 10 | 74 | 7.4 ± 1 |
| 66 | 4.2 | 65 | 14.7 ± 0.9 |
| 38 | 13 | 4 | 231 ± 13 |
| 83 | 5 | 10 | 119 ± 42.5 |

^{a}H-Phe-Met-Aib-Pmp-(6-Cl-Trp)-Glu-Ac₃c-Leu-NH₂.
^{b}Test compound concentration at which the p53 induction was maximal.
^{c}Compared to basal luciferase activity (16-h time point).
^{d}As measured in the fluorescence polarisation assay described in Example 3.
^{e}Highest concentration tested.

TABLE 4

IC₅₀ (μM)^{a}

| | Non- | |
|---|---|---|
| | synchronised | Synchronised |
| Compound | cells | cells^{b} |
| 2 | 23.2 ± 4.0 | n.d. |
| 3 | 28.8 ± 2.3 | 69 ± 30 |
| 5 | 73.3 ± 33.4 | 72 ± 31 |
| 6 | 37.5 ± 12.9 | 39 |
| 41 | 76.6 | 84 |
| 39 | 90.0 | n.d. |
| 40 | 99.5 | n.d. |
| 46 | 25.5 ± 3.1 | n.d. |
| 48 | 31.3 ± 2.6 | n.d. |
| 49 | 36.3 ± 2.9 | n.d. |
| 60 | 29.2 ± 6.8 | n.d. |
| 61 | 34.6 ± 22.9 | n.d. |
| 64 | 44.6 ± 2.3 | n.d. |
| 83 | 29.4 ± 6.0 | n.d. |

^{a}After 3-h treatment with test compounds.
^{b}Synchronisation with 0.3 mM mimosine for 24 h, followed by wash-out.

TABLE 5

| | IC₅₀ (μM) | | |
|---|---|---|---|
| Administration sequence | AGS | H1299 | SJSA-1 |
| 2 alone | 1.1 | 5.1 | 14 |
| Cisplatin alone | 3.8 | 2.9 | 6.1 |
| 2 + cisplatin simultaneously | 0.11 | 0.11 | 0.11 |
| Cisplatin + 2 after 6 h | 1.5 | 0.03 | 0.23 |
| 2 + cisplatin 6 h later | 0.21 | 0.18 | 0.02 |

TABLE 6

| | IC₅₀ (μM) | | |
|---|---|---|---|
| Administration sequence | AGS | H1299 | SJSA-1 |
| 2 alone | 1.1 | 5.1 | 14 |
| Etoposide alone | 0.28 | 1.3 | 16 |
| 2 + etoposide simultaneously | 0.20 | 0.04 | 0.19 |
| Etoposide + 2 6 h later | 0.25 | 0.06 | 1.1 |
| 2 + etoposide 6 h later | 0.08 | 0.05 | 0.02 |

1. A method of treating a proliferative disorder, comprising administering to a subject an effective amount of a compound of formula I, 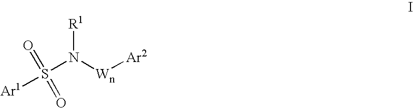
wherein
W is a C₁₋₅ branched or unbranched alkylene group or a C₂₋₅ alkenylene group;
n is 0 or 1;
R¹ is H, a C₁₋₈ branched or unbranched alkyl group, a C₂₋₈ alkenyl group, or an aryl or aralkyl group, each of which may be optionally substituted by one or more halogen or CF₃ groups;
Ar¹ is 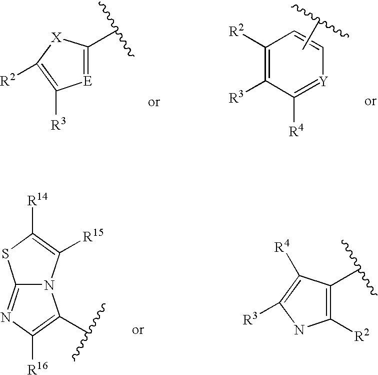
wherein
X is S, O, NH or NR′ where R′ is a C₁₋₃ alkyl group;
Y is CH or N;
E is N or CR⁴;
R², R³, R⁴, and R¹⁴⁻¹⁶ are each independently (A)ₚB, wherein A is C₁₋₃ alkyl, p is 0 or 1, and B is H, halogen, C₁₋₅ alkyl, NO₂, OH, NH₂, NHR^{a}, NR^{b}R^{c}, SO₃H, SO₂NH₂, NHAr^{a}, SO₂NHAr^{b}, SO₂NHR^{d}, SO₂Ar^{c}, SO₂R^{e}, CF₃, CN, COOH, COOR^{f}, CONH₂, COONHAr^{d}, CONHR^{g}, COAr^{e}, COR^{h}, S(CO)R^{s}, OR^{t}, OAr^{f}, an alicyclic group optionally containing one or more heteroatoms, optionally substituted by one or more OH, COR^{u}, halogen or CF₃ groups, or a heteroaryl group optionally substituted by one or more C₁₋₅ alkyl, halogen, SRⁱ or CF₃ groups; or
R² and R³ are linked to form a saturated or unsaturated ring system, optionally containing one or more heteroatoms, and optionally substituted by one or more halogen, OH or CF₃ groups;
Ar^{a-f} are each independently aryl groups optionally substituted by one or more C₁₋₅ alkyl, halogen or CF₃ groups;
R^{a-i}, R^{s}, R^{t} and R^{u} are each independently C₁₋₅ alkyl groups optionally substituted by one or more alkoxy, halogen or CF₃ groups;
and with the proviso that at least one of R², R³ and R⁴ is other than H;
Ar² is 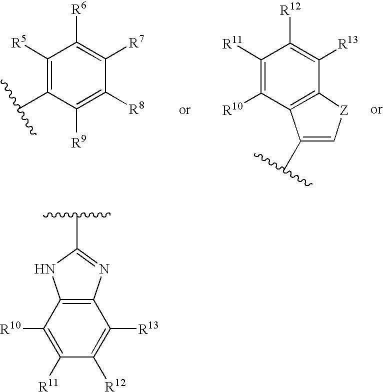
wherein
Z is S, O, NH or NR″ where R″ is C₁₋₃ alkyl;
R⁵, R⁶, R⁷, R⁸ and R⁹ are each independently (L)_{q}M wherein L is C₁₋₃ alkyl, q is 0 or 1, M is H, C₁₋₅ alkyl, halogen, NO₂, OH, NH₂, NHAr^{g}, NHR^{j}, NR^{k}R^{l}, SO₃H, SO₂NH₂, SO₂NHAr^{h}, SO₂NHR^{m}, SO₂Arⁱ, SO₂Rⁿ, CF₃, CN, COOH, COOR^{p}, CONH₂, CONHAr^{j}, CONHR^{q}, OR^{v}, COAr^{k} or COR^{r};
R^{j-r}, R^{v} are each independently C₁₋₅ alkyl groups;
Ar^{g-k} are each independently aryl groups;
and with the proviso that at least one of the substituents R⁵, R⁶, R⁷, R⁸ and R⁹ is other than H;
R¹⁰, R¹¹, R¹² and R¹³ are each independently H, C₁₋₅ alkyl, halogen, NO₂, OH, NH₂ or CF₃, or pharmaceutically acceptable salts, esters, or prodrugs thereof,
such that said subject is treated for said proliferative disorder.

2. The method according to claim 1, wherein Ar¹ is 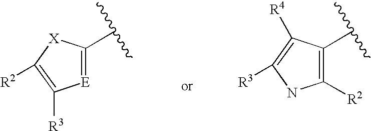
and Ar² is

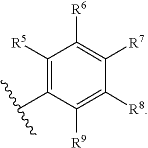

3. The method according to claim 1, wherein Ar¹ is 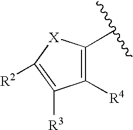
and Ar² is

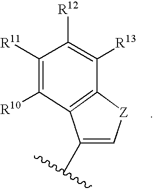

4. The method according to claim 1, wherein Ar¹ is 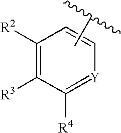
and Ar² is

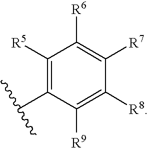

5. The method according to claim 1, wherein Ar¹ is 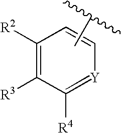
and Ar² is

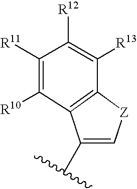

6. The method according to claim 1, wherein Ar¹ is 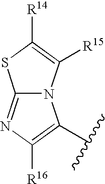
and Ar² is

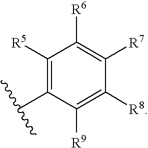

7. The method according to claim 1, wherein Ar¹ is 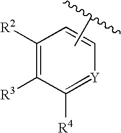
and Ar² is

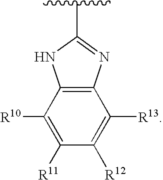

8. The method according to claim 1, wherein
R², R³ and R⁴ are each independently (A)ₚB, wherein A is C₁₋₅ alkyl, p is 0 or 1, and B is H, F, Cl, Br, I, C₁₋₅ alkyl, NO₂, OH, NH₂, NHR^{a}, NR^{b}R^{c}, SO₃H, SO₂NH₂, NHPh, SO₂NHAr^{b}, SO₂NHR^{d}, SO₂Ph, SO₂R^{e}, CF₃, CN, COOH, COOR^{f}, CONH₂, COONHPh, CONHR^{g}, S(CO)R^{s}, OR^{t}, OAr^{f}, COPh, COR^{h}, a morpholino, piperazino or piperidino group each of which may be optionally substituted by one or more OH or COR^{u} groups, or a heteroaryl group selected from pyridyl, pyrimidyl, oxazolyl, thiazolyl and pyrazolyl, each of which may be optionally substituted by one or more C₁₋₅ alkyl, halogen, SRⁱ or CF₃ groups, or R² and R³ together form a saturated 6-membered ring or an unsaturated 5-membered ring, each of which optionally contain one or more heteroatoms; and
R⁵, R⁶, R⁷, R⁸, and R⁹ are each independently (L)_{q}M wherein L is C₁₋₅ alkyl, q is 0 or 1, M is H, C₁₋₅ alkyl, halogen, NO₂, OH, NH₂, NHPh, NHR^{j}, NR^{k}R^{l}, SO₃H, SO₂NH₂, SO₂NHPh, SO₂NHR^{m}, SO₂Ph, SO₂Rⁿ, CF₃, CN, COOH, COOR^{p}, CONH₂, CONHPh, CONHR^{q}, OR^{v}, COPh or COR^{r}.

9. The method according to claim 1, wherein R², R³ and R⁴ are each independently (A)ₚB, wherein A is C₁₋₅ alkyl, p is 0 or 1, and B is H, F, Cl, Br, I, C₁₋₅ alkyl, NO₂, OH, NH₂, NHR^{a}, NR^{b}R^{c}, SO₃H, SO₂NH₂, NHPh, SO₂NHPh, SO₂NHR^{d}, SO₂Ph, SO₂R^{e}, CF₃, CN, COOH, COOR^{f}, CONH₂, COONHPh, CONHR^{g}, S(CO)R^{s}, OR^{t}, OAr^{f}, COPh, COR^{h}, pyridyl, pyrimidyl, 2-methylsulfanylpyrimid-5-yl, oxazol-2-yl, thiazol-2-yl, 1-methyl-5-trifluoromethyl-1H-pyrazol-4-yl, morpholin-4-yl, 4-acetyl-piperazin-1-yl, 3-hydroxy-piperidin-1-yl, or R² and R³ together form
—OCH₂CH₂O—
—N—S—N— or
a phenyl group optionally substituted by one or more halogens.

10. The method according to claim 1, wherein R², R³ and R⁴ are each independently H, halogen, NO₂, SO₂Ph, S(CO)Me, COOH, COOEt, OPh, OMe, NHCH₂CH₂OMe, 1-methyl-5-trifluoromethyl-1H-pyrazol-4-yl, 2-methylsulfanyl pyrimid-5-yl, N-(4-fluorophenyl)sulfonamido, N-(4-trifluoromethylphenyl)-sulfonamido, oxazol-2-yl, C₁₋₅ alkyl, NH₂, morpholin-4-yl, 4-acetyl-piperazin-1-yl, 3-hydroxy-piperidin-1-yl, or R² and R³ together form
—OCH₂CH₂O—
—N—S—N— or
a phenyl group optionally substituted by one or more halogens.

11. The method according to claim 1 wherein Ar¹ is 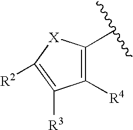
X is S or N;
R², R³ and R⁴ are each independently C₁₋₅ alkyl, S(CO)Me, COOH, NHCH₂CH₂OMe, COOEt, H, halogen, NO₂, SO₂Ph, SO₂NH-(4-chlorophenyl), 1-methyl-5-trifluoromethyl-1H-pyrazol-4-yl, morpholin-4-yl, 2-methylsulfanylpyrimid-5-yl, N-(4-fluorophenyl)sulfonamido, N-(4-trifluoro-methylphenyl)-sulfonamido, 3-hydroxy-piperidin-1-yl, pyridin-2-yl; or R² and R³ form a phenyl group optionally substituted by one or more halogens.

12. The method according to claim 11, wherein
R² is halogen, SO₂Ph, NO₂, Et, SOMe, morpholin-4-yl, NHCH₂CH₂OMe, 3-hydroxy-piperidin-1-yl, 1-methyl-5-trifluoromethyl-1H-pyrazol-4-yl or 2-methylsulfanyl-pyrimid-5-yl;
R³ is halogen, SO₂NH-(4-chlorophenyl), H, NO₂, N-(4-fluorophenyl)sulfonamido or N-(4-trifluoro methylphenyl)-sulfonamido; and
R⁴ is H.

13. The method according to claim 1 wherein Ar¹ is 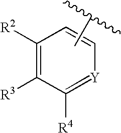
Y is CH or N; and
R², R³ and R⁴ are each independently H, OH, COOH, CF₃, OPh, OMe, NO₂, 4-acetyl-piperazin-1-yl, NH₂, halogen, pyrazol-1-yl, oxazol-2-yl or C₁₋₅ alkyl, or R² and R³ together form —OCH₂CH₂O— or —N—S—N—.

14. The method according to claim 13, wherein
when Y is CH
R² is H, NO₂ or Cl;
R³ is NO₂, NH₂, Cl, CF₃, COOH, 4-acetyl-piperazin-1-yl; and
R⁴ is H, Cl, oxazol-2-yl, OH, NO₂, NH₂, OMe or Me; or
when Y is N
R² is H;
R³ is Br;
R⁴ is Cl or OPh.

15. The method according to claim 1, wherein Ar¹ is 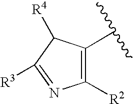
R² and R⁴ are C₁₋₅ alkyl, and R³ is COOH or COOEt.

16. The method according to claim 1, wherein
R⁵, R⁶, R⁷, R⁸ and R⁹ are each independently H, halogen, OMe, NO₂, C₁₋₅ alkyl, CF₃ or OH; and
R¹⁰, R¹¹, R¹² and R¹³ are all H.

17. The method according to claim 16 wherein
R⁵ is H, C₁₋₅ alkyl, or halogen;
R⁶ is H, halogen, NO₂, or CF₃;
R⁷ is H, halogen, OMe, NO₂, OH or CF₃;
R⁸ is H, halogen or CF₃;
R⁹ is H.

18. The method according to claim 1 wherein
W is CH₂, CH₂CH₂ or CH(CH₃)CH₂; and
R¹ is H, CH₂Ph, CH₂CH(Me)₂, 3-(trifluoromethyl)benzyl, or Me.

19. The method according to claim 1, wherein said compound of formula I is selected from
5-Chloro-4-nitrothiophene-2-sulfonic acid (3-trifluoromethylphenyl)amide [1];
5-Chloro-4-nitrothiophene-2-sulfonic acid (4-chlorophenyl)-amide [2];
5-Chloro-4-nitrothiophene-2-sulfonic acid (4-fluorophenyl)amide [3];
4-Bromo-5-chlorothiophene-2-sulfonic acid (4-fluorophenyl)amide [4];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
4,5-Dibromothiophene-2-sulfonic acid (3,5-bis-trifluoromethylphenyl)amide [8];
5-Chlorothiophene-2-sulfonic acid (4-trifluoromethylphenyl)amide [9];
5-Chlorothiophene-2-sulfonic acid (4-chlorophenyl)amide [10];
5-Chlorothiophene-2-sulfonic acid (3,5-bis-trifluoromethylphenyl)amide [11];
5-(2-Methylsulfanyl-pyrimidin-5-yl)-thiophene-2-sulfonic acid (3,5-dichlorophenyl)-amide [12];
4-Oxazol-2-yl-N-(4-trifluoromethylphenyl)benzenesulfonamide [13];
N-(3,5-Bis-trifluoromethylphenyl)-4-oxazol-2-yl-benzenesulfonamide [14];
4-Bromo-5-chlorothiophene-2-sulfonic acid (4-trifluoromethylphenyl)amide [15];
5-Bromothiophene-2-sulfonic acid (4-chlorophenyl)amide [16];
5-Bromothiophene-2-sulfonic acid (3,5-dichlorophenyl)amide [17];
5-Bromothiophene-2-sulfonic acid (3,5-bis-trifluoromethylphenyl)amide [18];
N-(4-Chlorophenyl)-3-nitrobenzenesulfonamide [19];
3-Nitro-N-(4-trifluoromethylphenyl)benzenesulfonamide [20];
N-(3,5-Bis-trifluoromethylphenyl)-3-nitrobenzenesulfonamide [21];
N-(2,4-Dichlorophenyl)-3-nitrobenzenesulfonamide [22];
5-Benzenesulfonylthiophene-2-sulfonic acid (4-trifluoromethylphenyl)-amide [23];
5-Benzenesulfonylthiophene-2-sulfonic acid (4-chlorophenyl)amide [24];
5-Benzenesulfonylthiophene-2-sulfonic acid (3,5-dichlorophenyl)amide [25];
5-Chlorothiophene-2-sulfonic acid (3,4-dichlorophenyl)amide [26];
4,5-Dibromothiophene-2-sulfonic acid (3-trifluoromethylphenyl)amide [27];
4,5-Dibromothiophene-2-sulfonic acid (3,4-dichlorophenyl)amide [28];
N-(3,5-Bis-trifluoromethylphenyl)-4-chloro-3-nitrobenzenesulfonamide [29];
4-Chloro-N-(3,4-dichlorophenyl)-3-nitrobenzenesulfonamide [30];
5-(1-Methyl-5-trifluoromethyl-1H-pyrazol-4-yl)-thiophene-2-sulfonic acid (4-trifluoro-methylphenyl)-amide [31];
5-Chlorothiophene-2,4-disulfonic acid bis-[(4-fluorophenyl)-amide] [32];
5-Chlorothiophene-2,4-disulfonic acid bis-[(4-trifluoro-methyl-phenyl)-amide] [33];
4-Methyl-3-nitro-N-(4-trifluoromethylphenyl)benzenesulfonamide [34];
4-Chloro-3-nitro-N-(4-trifluoromethylphenyl)benzenesulfonamide [35];
3-Amino-4-methyl-N-(4-trifluoromethyl-phenyl)benzenesulfonamide [36];
N-(4-Chlorophenyl)-4-methyl-3-nitrobenzenesulfonamide [37];
4-Chloro-N-(4-chlorophenyl)-3-nitro-benzenesulfonamide [38];
5-Chloro-4-nitrothiophene-2-sulfonic acid (3,5-dichlorophenyl)-amide [39];
5-Chloro-4-nitrothiophene-2-sulfonic acid (3,5-difluorophenyl)-amide [40];
5-Bromo-6-chloropyridine-3-sulfonic acid (4-trifluoromethylphenyl)amide [41];
5-Bromo-6-chloropyridine-3-sulfonic acid (3,5-bis-trifluoromethylphenyl)amide [42];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethyl-phenyl)-amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
4-Chloro-N-(3,5-dichlorophenyl)-3-nitro-benzenesulfonamide [47];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid methyl-(4-trifluoromethylphenyl)amide [50];
5-Chloro-4-nitrothiophene-2-sulfonic acid (4-chloro-phenyl)-methylamide [51]; and
5-Chloro-4-nitrothiophene-2-sulfonic acid methyl-(4-trifluoromethylbenzyl)amide [52];
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (4-chloro-phenyl)-isobutyl-amide [58];
5-Chloro-4-nitro-thiophene-2-sulfonic acid (1H-benzo-imidazol-2-yl)-amide [59];
5-Chloro-4-nitro-thiophene-2-sulfonic acid [2-(6-chloro-1H-indol-3-yl)-ethyl]-amide [60];
5-Chloro-4-nitro-thiophene-2-sulfonic acid (4-methoxy-phenyl)-amide [61];
5-Chloro-4-nitro-thiophene-2-sulfonic acid p-tolylamide [63];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
5-Nitro-thiophene-2-sulfonic acid (4-chloro-phenyl)-amide [68];
4-Nitro-thiophene-2-sulfonic acid (4-chloro-phenyl)-amide [69];
5-Chloro-thiophene-2,4-disulfonic acid bis-[(4-chloro-phenyl)-amide] [70];
5-Ethyl-4-nitro-thiophene-2-sulfonic acid (4-chloro-phenyl)-amide [71];
Thioacetic acid S-[5-(4-chloro-phenylsulfamoyl)-3-nitro-thiophen-2-yl]ester [72];
5-Methyl-4-nitro-thiophene-2-sulfonic acid (4-chloro-phenyl)-amide [73];
5-Methyl-thiophene-2,4-disulfonic acid bis-[(4-chloro-phenyl)-amide] [74];
5-Chloro-4-nitro-thiophene-2-sulfonic acid (3-trifluoro-methyl-benzyl)-(4-trifluoromethyl-benzyl)-amide [75];
4-Nitro-thiophene-2-sulfonic acid (4-trifluoromethyl-phenyl)-amide [76];
4-Nitro-thiophene-2-sulfonic acid [2-(1H-indol-3-yl)-ethyl]-amide [77];
5-(1-Methyl-5-tri-fluoromethyl-1H-pyrazol-3-yl)-thiophene-2-sulfonic acid (3,5-bis-trifluoromethyl-phenyl)-(3-trifluoro-methyl-benzyl)-amide [78];
5-Morpholin-4-yl-4-nitro-thiophene-2-sulfonic acid (4-chloro-phenyl)-amide [79];
5-(2-Methoxy-ethylamino)-4-nitro-thiophene-2-sulfonic acid (4-chloro-phenyl)-amide [80];
4-Chloro-N-[2-(5-chloro-1H-indol-3-yl)-ethyl]-3-nitro-benzenesulfonamide [81];
N-[2-(5-Chloro-1H-indol-3-yl)-ethyl]-4-methyl-3-nitro-benzenesulfonamide [82];
N-(1H-Benzoimidazol-2-yl)-4-chloro-3-nitro-benzenesulfonamide [83];
6-Chloro-imidazo[2,1-b]thiazole-5-sulfonic acid (3,5-bis-trifluoro-methyl-phenyl)-amide [84];
2,3-Dihydro-benzo[1,4]dioxine-6-sulfonic acid (4-chloro-phenyl)-amide [85];
2,3-Dihydro-benzo[1,4]dioxine-6-sulfonic acid (3,5-bis-trifluoromethyl-phenyl)-amide [86];
6-Phenoxy-pyridine-3-sulfonic acid (4-chloro-phenyl)-amide [87];
5-Chloro-3-methyl-benzo[b]thiophene-2-sulfonic acid (4-chloro-3-nitro-phenyl)-amide [88];
N-(3,5-Bis-trifluoromethyl-phenyl)-4-pyrazol-1-yl-benzenesulfonamide [89];
4-(4-Chloro-phenylsulfamoyl)-3,5-dimethyl-1H-pyrrole-2-carboxylic acid ethyl ester [90];
4-(3,5-Bis-trifluoromethyl-phenylsulfamoyl)-3,5-dimethyl-1H-pyrrole-2-carboxylic acid [91];
4-(4-Chloro-phenylsulfamoyl)-3,5-dimethyl-1H-pyrrole-2-carboxylic acid [92];
2-(4-Chloro-phenylsulfamoyl)-4-methyl-thiazole-5-carboxylic acid ethyl ester [93];
3,5-Dichloro-N-(4-chloro-phenyl)-4-hydroxy-benzenesulfonamide [94];
N-(3,5-Bis-trifluoromethyl-phenyl)-3,5-dichloro-4-hydroxy-benzenesulfonamide [95];
3,5-Dichloro-4-hydroxy-N-(4-trifluoromethyl-phenyl)-benzenesulfonamide [96];
N-(4-Chloro-phenyl)-4-nitro-benzene-sulfonamide [97];
N-(3,5-Bis-trifluoromethyl-phenyl)-4-nitro-benzenesulfonamide [98];
4-Amino-N-(3,5-bis-trifluoromethyl-phenyl)-3-chloro-benzenesulfonamide [99];
3-Nitro-N-(4-trifluoromethyl-phenyl)-benzenesulfonamide [100];
3,5-Dichloro-N-(3,5-dichloro-phenyl)-4-hydroxy-benzenesulfonamide [101];
4-Amino-3-chloro-N-(4-chloro-phenyl)-benzenesulfonamide [102];
3-Chloro-N-(4-chloro-phenyl)-4-methoxy-benzenesulfonamide [103];
N-(3,5-Bis-trifluoromethyl-phenyl)-3-chloro-4-methoxy-benzene-sulfonamide [104];
N-(3-Chloro-4-nitro-phenyl)-3,5-bis-trifluoromethyl-benzenesulfonamide [105];
3-(4-Acetyl-piperazin-1-yl)-N-(3,5-bis-trifluoromethyl-phenyl)-4-nitro-benzenesulfonamide [106];
N-(3,5-Bis-trifluoromethyl-phenyl)-2-nitro-benzenesulfonamide [107];
3-(3,5-Bis-trifluoromethyl-phenylsulfamoyl)-benzoic acid [108];
3,5-Dichloro-N-(4-chloro-benzyl)-4-hydroxy-benzenesulfonamide [109];
3,5-Dichloro-4-hydroxy-N-(4-trifluoromethyl-benzyl)-benzenesulfonamide [110];
3,5-Dichloro-4-hydroxy-N-[2-(1H-indol-3-yl)-ethyl]-benzenesulfonamide [111];
4,5-Dibromo-thiophene-2-sulfonic acid (3,5-dichloro-phenyl)-amide [112];
N-(3,5-Dichloro-phenyl)-4-oxazol-2-yl-benzenesulfonamide [113];
4-Bromo-5-chloro-thiophene-2-sulfonic acid (3,5-bis-trifluoromethyl-phenyl)-amide [114];
4-Bromo-5-chloro-thiophene-2-sulfonic acid (3,5-dichloro-phenyl)-amide [115];
5-Bromo-thiophene-2-sulfonic acid (4-trifluoromethyl-phenyl)-amide [116];
5-Benzenesulfonyl-thiophene-2-sulfonic acid (3,5-bis-trifluoromethyl-phenyl)-amide [117];
5-Benzenesulfonyl-thiophene-2-sulfonic acid (2,4-dichloro-phenyl)-amide [118];
5-Chloro-3-methyl-benzo[b]thiophene-2-sulfonic acid (3,5-bis-trifluoromethyl-phenyl)-amide [119];
Benzo[b]thiophene-2-sulfonic acid (3,5-bis-trifluoromethyl-phenyl)-amide [120];
Benzo[1,2,5]thiadiazole-5-sulfonic acid (4-chloro-phenyl)-amide [121];
Benzo[1,2,5]thiadiazole-5-sulfonic acid (3,5-bis-trifluoromethyl-phenyl)-amide [122];
Benzo[1,2,5]thiadiazole-5-sulfonic acid (4-trifluoromethyl-phenyl)-amide [123];
5-Pyridin-2-yl-thiophene-2-sulfonic acid (3,5-bis-trifluoromethyl-phenyl)-amide [124];
4,5-Dibromo-thiophene-2-sulfonic acid (4-chloro-phenyl)-amide [125];
4,5-Dibromo-thiophene-2-sulfonic acid (4-trifluoromethyl-phenyl)-amide [126];
3,5-Dichloro-N-(4-fluoro-benzyl)-4-hydroxy-benzenesulfonamide [127];
N-(3,5-Bis-trifluoromethyl-phenyl)-2,6-dichloro-benzenesulfonamide [128];
5-Chloro-4-nitro-thiophene-2-sulfonic acid (4-methoxy-2-methyl-phenyl)-amide [129];
5-(3-Hydroxy-piperidin-1-yl)-4-nitro-thiophene-2-sulfonic acid (4-chloro-phenyl)-amide [130]; and
5-Chloro-4-nitro-thiophene-2-sulfonic acid (4-nitro-phenyl)-amide [131].

20. The method according to claim 19 wherein the compound of formula I is selected from the following:
5-Chloro-4-nitrothiophene-2-sulfonic acid (4-chlorophenyl)amide [2];
5-Chloro-4-nitrothiophene-2-sulfonic acid (4-fluorophenyl)amide [3];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
4-Chloro-3-nitro-N-(4-trifluoromethylphenyl)-benzene-sulfonamide [35];
4-Chloro-N-(4-chlorophenyl)-3-nitrobenzene-sulfonamide [38];
5-Chloro-4-nitrothiophene-2-sulfonic acid (3,5-dichlorophenyl)amide [39];
5-Chloro-4-nitrothiophene-2-sulfonic acid (3,5-difluorophenyl)amide [40];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethyl-phenyl)-amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
4-Chloro-N-(3,5-dichlorophenyl)-3-nitro-benzenesulfonamide [47];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid (4-chlorophenyl)methylamide [51];
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (4-methoxy-phenyl)-amide [61];
5-Chloro-4-nitro-thiophene-2-sulfonic acid p-tolylamide [63];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
4-Nitro-thiophene-2-sulfonic acid (4-chloro-phenyl)-amide [69];
5-Chloro-4-nitro-thiophene-2-sulfonic acid (3-trifluoro-methyl-benzyl)-(4-trifluoromethyl-benzyl)-amide [75]; and
N-(1H-Benzoimidazol-2-yl)-4-chloro-3-nitro-benzenesulfonamide [83].

21. The method according to claim 19 wherein the compound of formula I is selected from the following:
5-Chloro-4-nitrothiophene-2-sulfonic acid (4-chlorophenyl)amide [2];
5-Chloro-4-nitrothiophene-2-sulfonic acid (4-fluorophenyl)amide [3];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
5-Chlorothiophene-2-sulfonic acid (4-trifluoromethylphenyl)amide [9];
5-Bromothiophene-2-sulfonic acid (3,5-bis-trifluoromethylphenyl)amide [18];
5-Benzenesulfonyl-thiophene-2-sulfonic acid (4-trifluoromethylphenyl)amide [23];
4,5-Dibromothiophene-2-sulfonic acid (3-trifluoromethylphenyl)amide [27];
4,5-Dibromothiophene-2-sulfonic acid (3,4-dichlorophenyl)amide [28];
N-(3,5-Bis-trifluoromethylphenyl)-4-chloro-3-nitrobenzene-sulfonamide [29];
5-Chlorothiophene-2,4-disulfonic acid bis-[(4-trifluoromethylphenyl)amide] [33];
4-Chloro-3-nitro-N-(4-trifluoromethylphenyl)benzene-sulfonamide [35];
5-Chloro-4-nitrothiophene-2-sulfonic acid (3,5-dichlorophenyl)amide [39];
5-Chloro-4-nitrothiophene-2-sulfonic acid (3,5-difluorophenyl)amide [40];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethylphenyl)amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid methyl-(4-trifluoromethylphenyl)amide [50];
5-Chloro-4-nitrothiophene-2-sulfonic acid (4-chlorophenyl)methylamide [51]; and
5-Chloro-4-nitrothiophene-2-sulfonic acid methyl (4-trifluoromethylbenzyl)amide [52]
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (1H-benzo-imidazol-2-yl)-amide [59];
5-Chloro-4-nitro-thiophene-2-sulfonic acid [2-(6-chloro-1H-indol-3-yl)-ethyl]-amide [60];
5-Chloro-4-nitro-thiophene-2-sulfonic acid (4-methoxy-phenyl)-amide [61];
5-Chloro-4-nitro-thiophene-2-sulfonic acid p-tolylamide [63];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
4-Nitro-thiophene-2-sulfonic acid (4-chloro-phenyl)-amide [69];
5-Chloro-thiophene-2,4-disulfonic acid bis-[(4-chloro-phenyl)-amide] [70];
Thioacetic acid S-[5-(4-chloro-phenylsulfamoyl)-3-nitro-thiophen-2-yl]ester [72];
5-Methyl-4-nitro-thiophene-2-sulfonic acid (4-chloro-phenyl)-amide [73];
5-Methyl-thiophene-2,4-disulfonic acid bis-[(4-chloro-phenyl)-amide] [74];
5-Chloro-4-nitro-thiophene-2-sulfonic acid (3-trifluoro-methyl-benzyl)-(4-trifluoromethyl-benzyl)-amide [75];
5-(2-Methoxy-ethylamino)-4-nitro-thiophene-2-sulfonic acid (4-chloro-phenyl)-amide [80];
N-(1H-Benzoimidazol-2-yl)-4-chloro-3-nitro-benzenesulfonamide [83];
5-Chloro-3-methyl-benzo[b]thiophene-2-sulfonic acid (4-chloro-3-nitro-phenyl)-amide [88];
N-(3,5-Bis-trifluoromethyl-phenyl)-3-chloro-4-methoxy-benzene-sulfonamide [104];
N-(3-Chloro-4-nitro-phenyl)-3,5-bis-trifluoromethyl-benzenesulfonamide [105];
4,5-Dibromo-thiophene-2-sulfonic acid (3,5-dichloro-phenyl)-amide [112];
5-Bromo-thiophene-2-sulfonic acid (4-trifluoromethyl-phenyl)-amide [116];
5-Benzenesulfonyl-thiophene-2-sulfonic acid (3,5-bis-trifluoromethyl-phenyl)-amide [117];
5-Chloro-3-methyl-benzo[b]thiophene-2-sulfonic acid (3,5-bis-trifluoromethyl-phenyl)-amide [119];
Benzo[b]thiophene-2-sulfonic acid (3,5-bis-trifluoromethyl-phenyl)-amide [120];
Benzo[1,2,5]thiadiazole-5-sulfonic acid (3,5-bis-trifluoromethyl-phenyl)-amide [122];
4,5-Dibromo-thiophene-2-sulfonic acid (4-chloro-phenyl)-amide [125];
4,5-Dibromo-thiophene-2-sulfonic acid (4-trifluoromethyl-phenyl)-amide [126];
N-(3,5-Bis-trifluoromethyl-phenyl)-2,6-dichloro-benzenesulfonamide [128];
5-(3-Hydroxy-piperidin-1-yl)-4-nitro-thiophene-2-sulfonic acid (4-chloro-phenyl)-amide [130]; and
5-Chloro-4-nitro-thiophene-2-sulfonic acid (4-nitro-phenyl)-amide [131].

22. The method according to claim 1, wherein the proliferative disorder is cancer.

23. The method according to claim 1, wherein said compound is administered in an amount sufficient to inhibit the interaction between HDM2 and p53 and/or HDM2 and E2F transcription factors.

24. A compound of formula Ia, 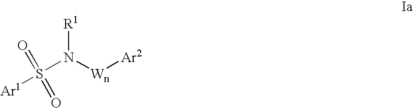
wherein
W is a C₁₋₅ branched or unbranched alkylene group or a C₂₋₅ alkenylene group;
n is 0 or 1;
R¹ is H, a C₁₋₈ branched or unbranched alkyl group, a C₂₋₈ alkenyl group, or an aryl or aralkyl group, each of which may be optionally substituted by one or more halogen or CF₃ groups; 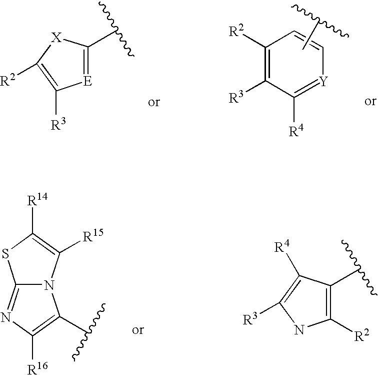
Ar¹ is
wherein
X is O, NH or NR′ where R′ is a C₁₋₃ alkyl group;
E is N or CR⁴;
Y is N;
R², R³, R⁴, and R¹⁴⁻¹⁶ are each independently (A)ₚB, wherein A is C₁₋₃ alkyl, p is 0 or 1, and B is H, halogen, C₁₋₅ alkyl, NO₂, OH, NH₂, NHR^{a}, NR^{b}R^{c}, SO₃H, SO₂NH₂, NHAr^{a}, SO₂NHAr^{b}, SO₂NHR^{d}, SO₂Ar^{c}, SO₂R^{e}, CF₃, CN, COOH, COOR^{f}, CONH₂, COONHAr^{d}, CONHR^{g}, COAr^{e}, COR^{h}, S(CO)R^{s}, OR^{t}, OAr^{f}, an alicyclic group optionally containing one or more heteroatoms, optionally substituted by one or more OH, COR^{u}, halogen or CF₃ groups, or a heteroaryl group optionally substituted by one or more C₁₋₅ alkyl, halogen, SRⁱ or CF₃ groups; or
R² and R³ are linked to form a saturated or unsaturated ring system, optionally containing one or more heteroatoms, and optionally substituted by one or more halogen, OH or CF₃ groups;
Ar^{a-f} are each independently aryl groups optionally substituted by one or more C₁₋₅ alkyl, halogen or CF₃ groups;
R^{a-i}, R^{s}, R^{t} and R^{u} are each independently C₁₋₅ alkyl groups optionally substituted by one or more alkoxy, halogen or CF₃ groups;
and with the proviso that at least one of R², R³ and R⁴ is other than H;
Ar² is 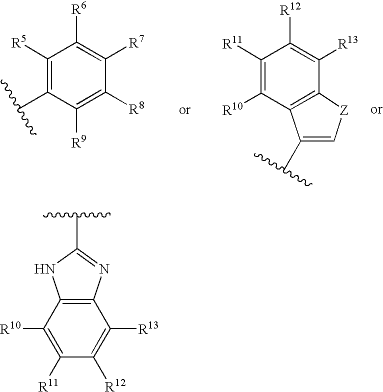
wherein
Z is S, O, NH or NR″ where R″ is C₁₋₃ alkyl;
R⁵, R⁶, R⁷, R⁸ and R⁹ are each independently (L)_{q}M wherein L is C₁₋₃ alkyl, q is 0 or 1, M is H, C₁₋₅ alkyl, halogen, NO₂, OH, NH₂, NHAr^{g}, NHR^{j}, NR^{k}R^{l}, SO₃H, SO₂NH₂, SO₂NHAr^{h}, SO₂NHR^{m}, SO₂Arⁱ, SO₂Rⁿ, CF₃, CN, COOH, COOR^{p}, CONH₂, CONHAr^{j}, CONHR^{q}, OR^{v}, COAr^{k} or COR^{r};
R^{j-r}, R^{v} are each independently C₁₋₅ alkyl groups;
Ar^{g-k} are each independently aryl groups;
and with the proviso that at least one of the substituents R⁵, R⁶, R⁷, R⁸ and R⁹ is other than H;
R¹⁰, R¹¹, R¹² and R¹³ are each independently H, C₁₋₅ alkyl, halogen, NO₂, OH, NH₂ or CF₃, or pharmaceutically acceptable salts, esters, or prodrugs thereof,
with the proviso that said compound is other than
5-[(4-chlorophenyl)amino]sulfonyl-2-furancarboxylic acid.

25. A compound according to claim 24 wherein Ar¹ is 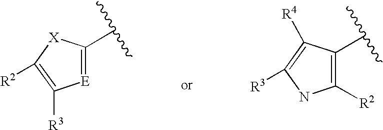
and Ar² is

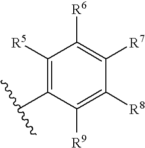

26. A compound according to claim 24 wherein Ar¹ is 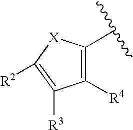
and Ar² is

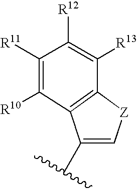

27. A compound according to claim 24 wherein Ar¹ is 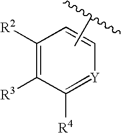
and Ar² is

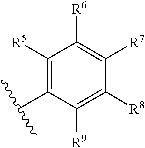

28. A compound according claim 24 wherein Ar¹ is 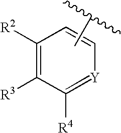
and Ar² is

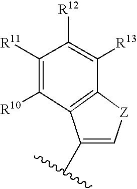

29. A compound according to claim 24 wherein Ar¹ is 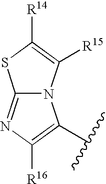
and Ar² is

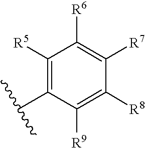

30. A compound according to claim 24 wherein Ar¹ is 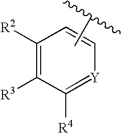
and Ar² is

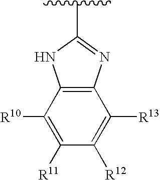

31. A compound of formula Ib, 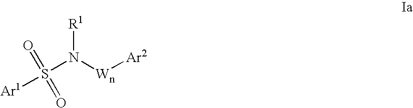
wherein
W is a C₁₋₅ branched or unbranched alkylene group or a C₂₋₅ alkenylene group;
n is 0 or 1;
R¹ is H, a C₁₋₈ branched or unbranched alkyl group, a C₂₋₈ alkenyl group, or an aryl or aralkyl group, each of which may be optionally substituted by one or more halogen or CF₃ groups;
Ar¹ is 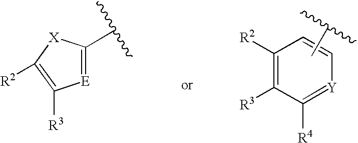
wherein
X is S, O, NH or NR′ where R′ is a C₁₋₃ alkyl group;
E is N or CR⁴;
Y is CH or N;
R², R³ and R⁴ are each independently (A)ₚB, wherein A is C₁₋₃ alkyl, p is 0 or 1, and B is H, halogen, C₁₋₅ alkyl, NO₂, OH, NH₂, NHR^{a}, NR^{b}R^{c}, SO₃H, SO₂NH₂, NHAr^{a}, SO₂NHAr^{b}, SO₂NHR^{d}, SO₂Ar^{c}, SO₂R^{e}, CF₃, CN, COOH, COOR^{f}, CONH₂, COONHAr^{d}, CONHR^{g}, COAr^{e}, COR^{h}, S(CO)R^{s}, OR^{t}, OAr^{f}, an alicyclic group optionally containing one or more heteroatoms, optionally substituted by one or more OH, COR^{u}, halogen or CF₃ groups, or a heteroaryl group optionally substituted by one or more C₁₋₅ alkyl, halogen, SRⁱ or CF₃ groups; or
R² and R³ are linked to form a saturated or unsaturated ring system, optionally containing one or more heteroatoms, and optionally substituted by one or more halogen, OH or CF₃ groups;
Ar^{a-f} are each independently aryl groups optionally substituted by one or more C₁₋₅ alkyl, halogen or CF₃ groups;
R^{a-i}, R^{s}, R^{t} and R^{u} are each independently C₁₋₅ alkyl groups optionally substituted by one or more alkoxy, halogen or CF3 groups;
and with the proviso that at least one of R², R³ and R⁴ is other than H;
Ar² is 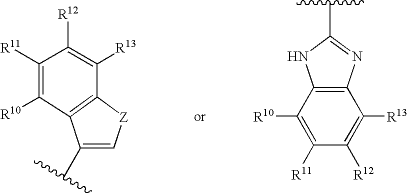
wherein
Z is S, O, NH or NR″ where R″ is C₁₋₃ alkyl;
R⁵, R⁶, R⁷, R⁸ and R⁹ are each independently (L)_{q}M wherein L is C₁₋₃ alkyl, q is 0 or 1, M is H, C₁₋₅ alkyl, halogen, NO₂, OH, NH₂, NHAr^{g}, NHR^{j}, NR^{k}R^{l}, SO₃H, SO₂NH₂, SO₂NHAr^{h}, SO₂NHR^{m}, SO₂Arⁱ, SO₂Rⁿ, CF₃, CN, COOH, COOR^{p}, CONH₂, CONHAr^{j}, CONHR^{q}, OR^{v}, COAr^{k} or COR^{r};
R^{j-r}, R^{v} are each independently C₁₋₅ alkyl groups;
Ar^{g-k} are each independently aryl groups;
and with the proviso that at least one of the substituents R⁵, R⁶, R⁷, R⁸ and R⁹ is other than H;
R¹⁰, R¹¹, R¹² and R¹³ are each independently H, C₁₋₅ alkyl, halogen, NO₂, OH, NH₂ or CF₃, or pharmaceutically acceptable salts, esters, or prodrugs thereof,

32. A compound according to claim 24 or 31 wherein
R², R³ and R⁴ are each independently (A)ₚB, wherein A is C₁₋₅ alkyl, p is 0 or 1, and B is H, F, Cl, Br, I, C₁₋₅ alkyl, NO₂, OH, NH₂, NHR^{a}, NR^{b}R^{c}, SO₃H, SO₂NH₂, NHPh, SO₂NHAr^{b}, SO₂NHR^{d}, SO₂Ph, SO₂R^{e}, CF₃, CN, COOH, COOR^{f}, CONH₂, COONHPh, CONHR^{g}, S(CO)R^{s}, OR^{t}, OAr^{f}, COPh, COR^{h}, a morpholino, piperazino or piperidino group each of which may be optionally substituted by one or more OH or COR^{u} groups, or a heteroaryl group selected from pyridyl, pyrimidyl, oxazolyl, thiazolyl and pyrazolyl, each of which may be optionally substituted by one or more C₁₋₅ alkyl, halogen, SRⁱ or CF₃ groups, or R² and R³ together form a saturated 6-membered ring or an unsaturated 5-membered ring, each of which optionally contain one or more heteroatoms; and
R⁵, R⁶, R⁷, R⁸, and R⁹ are each independently (L)_{q}M wherein L is C₁₋₅ alkyl, q is 0 or 1, M is H, C₁₋₅ alkyl, halogen, NO₂, OH, NH₂, NHPh, NHR^{j}, NR^{k}R^{l}, SO₃H, SO₂NH₂, SO₂NHPh, SO₂NHR^{m}, SO₂Ph, SO₂Rⁿ, CF₃, CN, COOH, COOR^{p}, CONH₂, CONHPh, CONHR^{q}, OR^{v}, COPh or COR^{r}.

33. A compound according to claims 24 or 31 wherein R², R³ and R⁴ are each independently (A)ₚB, wherein A is C₁₋₅ alkyl, p is 0 or 1, and B is H, F, Cl, Br, I, C₁₋₅ alkyl, NO₂, OH, NH₂, NHR^{a}, NR^{b}R^{c}, SO₃H, SO₂NH₂, NHPh, SO₂NHPh, SO₂NHR^{d}, SO₂Ph, SO₂R^{e}, CF₃, CN, COOH, COOR^{f}, CONH₂, COONHPh, CONHR^{g}, S(CO)R^{s}, OR^{t}, OAr^{f}, COPh, COR^{h}, pyridyl, pyrimidyl, 2-methylsulfanylpyrimid-5-yl, oxazol-2-yl, thiazol-2-yl, 1-methyl-5-trifluoromethyl-1H-pyrazol-4-yl, morpholin-4-yl, 4-acetyl-piperazin-1-yl, 3-hydroxy-piperidin-1-yl, or R² and R³ together form
—OCH₂CH₂O—
—N—S—N— or
a phenyl group optionally substituted by one or more halogens.

34. A compound according to claims 24 or 31 wherein R², R³ and R⁴ are each independently H, halogen, NO₂, SO₂Ph, S(CO)Me, COOH, COOEt, OPh, OMe, NHCH₂CH₂OMe, 1-methyl-5-trifluoromethyl-1H-pyrazol-4-yl, 2-methylsulfanylpyrimid-5-yl, N-(4-fluorophenyl)sulfonamido, N-(4-trifluoromethylphenyl)sulfonamido, oxazol-2-yl, C₁₋₅ alkyl, NH₂, morpholin-4-yl, 4-acetyl-piperazin-1-yl, 3-hydroxy-piperidin-1-yl, or R² and R³ together form
OCH₂CH₂O—
—N—S—N— or
a phenyl group optionally substituted by one or more halogens.

35. A compound according to claim 24 wherein Ar¹ is 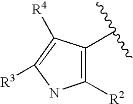
R² and R⁴ are C₁₋₅ alkyl, and R³ is COOH or COOEt.

36. A compound according to claim 24 or 31 wherein Ar¹ is 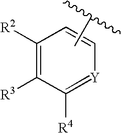
R², R³ and R⁴ are each independently H, OH, COOH, CF₃, OPh, OMe, NO₂, 4-acetyl-piperazin-1-yl, NH₂, halogen, pyrazol-1-yl, oxazol-2-yl or C₁₋₅ alkyl, or R² and R³ together form —OCH₂CH₂O— or —N—S—N—.

37. A compound according to claim 36 wherein
when Y is CH
R² is H, NO₂ or Cl;
R³ is NO₂, NH₂, Cl, CF₃, COOH, 4-acetyl-piperazin-1-yl; and
R⁴ is H, Cl, oxazol-2-yl, OH, NO₂, NH₂, OMe or Me; or
when Y is N
R² is H;
R³ is Br;
R⁴ is Cl or OPh.

38. A compound according to claim 24 or 31 wherein
R⁵, R⁶, R⁷, R⁸ and R⁹ are each independently H, halogen, OMe, NO₂, C₁₋₅ alkyl, CF₃ or OH; and
R¹⁰, R¹¹, R¹² and R¹³ are all H.

39. A compound according to claim 38 wherein
R⁵ is H, C₁₋₅ alkyl, or halogen;
R⁶ is H, halogen, NO₂, or CF₃;
R⁷ is H, halogen, OMe, NO₂, OH or CF₃;
R⁸ is H, halogen or CF₃;
R⁹ is H.

40. A compound according to claim 24 or 31 wherein
W is CH₂, CH₂CH₂ or CH(CH₃)CH₂; and
R¹ is H, CH₂Ph, CH₂CH(Me)₂, 3-(trifluoromethyl)benzyl, or Me.

41. A compound selected from the following:
5-Chloro-4-nitrothiophene-2-sulfonic acid (3-trifluoromethylphenyl)amide [1];
5-Chloro-4-nitrothiophene-2-sulfonic acid (4-chlorophenyl)-amide [2];
5-Chloro-4-nitrothiophene-2-sulfonic acid (4-fluorophenyl)amide [3];
4-Bromo-5-chlorothiophene-2-sulfonic acid (4-fluorophenyl)amide [4];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
4,5-Dibromothiophene-2-sulfonic acid (3,5-bis-trifluoromethylphenyl)amide [8];
5-Chlorothiophene-2-sulfonic acid (3,5-bis-trifluoromethylphenyl)amide [11];
5-(2-Methylsulfanyl-pyrimidin-5-yl)-thiophene-2-sulfonic acid (3,5-dichlorophenyl)-amide [12];
4-Oxazol-2-yl-N-(4-trifluoromethylphenyl)benzenesulfonamide [13];
N-(3,5-Bis-trifluoromethylphenyl)-4-oxazol-2-yl-benzenesulfonamide [14];
4-Bromo-5-chlorothiophene-2-sulfonic acid (4-trifluoromethylphenyl)amide [15];
5-Bromothiophene-2-sulfonic acid (4-chlorophenyl)amide [16];
5-Bromothiophene-2-sulfonic acid (3,5-dichlorophenyl)amide [17];
5-Bromothiophene-2-sulfonic acid (3,5-bis-trifluoromethylphenyl)amide [18];
N-(4-Chlorophenyl)-3-nitrobenzenesulfonamide [19];
3-Nitro-N-(4-trifluoromethylphenyl)benzenesulfonamide [20];
N-(3,5-Bis-trifluoromethylphenyl)-3-nitrobenzenesulfonamide [21];
N-(2,4-Dichlorophenyl)-3-nitrobenzenesulfonamide [22];
5-Benzenesulfonylthiophene-2-sulfonic acid (4-trifluoromethylphenyl)-amide [23];
5-Benzenesulfonylthiophene-2-sulfonic acid (4-chlorophenyl)amide [24];
5-Benzenesulfonylthiophene-2-sulfonic acid (3,5-dichlorophenyl)amide [25];
5-Chlorothiophene-2-sulfonic acid (3,4-dichlorophenyl)amide [26];
4,5-Dibromothiophene-2-sulfonic acid (3-trifluoromethylphenyl)amide [27];
4,5-Dibromothiophene-2-sulfonic acid (3,4-dichlorophenyl)amide [28];
4-Chloro-N-(3,4-dichlorophenyl)-3-nitrobenzenesulfonamide [30];
5-(1-Methyl-5-trifluoromethyl-1H-pyrazol-4-yl)-thiophene-2-sulfonic acid (4-trifluoromethylphenyl)-amide [31];
5-Chlorothiophene-2,4-disulfonic acid bis-[(4-fluorophenyl)-amide] [32];
5-Chlorothiophene-2,4-disulfonic acid bis-[(4-trifluoro-methyl-phenyl)-amide] [33];
4-Methyl-3-nitro-N-(4-trifluoromethylphenyl)benzenesulfonamide [34];
4-Chloro-3-nitro-N-(4-trifluoromethylphenyl)benzenesulfonamide [35];
3-Amino-4-methyl-N-(4-trifluoromethyl-phenyl)benzenesulfonamide [36];
N-(4-Chlorophenyl)-4-methyl-3-nitrobenzenesulfonamide [37];
4-Chloro-N-(4-chlorophenyl)-3-nitro-benzenesulfonamide [38];
5-Chloro-4-nitrothiophene-2-sulfonic acid (3,5-dichlorophenyl)-amide [39];
5-Chloro-4-nitrothiophene-2-sulfonic acid (3,5-difluorophenyl)-amide [40];
5-Bromo-6-chloropyridine-3-sulfonic acid (4-trifluoromethylphenyl)amide [41];
5-Bromo-6-chloropyridine-3-sulfonic acid (3,5-bis-trifluoromethylphenyl)amide [42];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethyl-phenyl)-amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
4-Chloro-N-(3,5-dichlorophenyl)-3-nitro-benzenesulfonamide [47];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid methyl-(4-trifluoromethylphenyl)amide [50];
5-Chloro-4-nitrothiophene-2-sulfonic acid (4-chloro-phenyl)-methylamide [51]; and
5-Chloro-4-nitrothiophene-2-sulfonic acid methyl-(4-trifluoromethylbenzyl)amide [52];
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (4-chloro-phenyl)-isobutyl-amide [58];
5-Chloro-4-nitro-thiophene-2-sulfonic acid (1H-benzo-imidazol-2-yl)-amide [59];
5-Chloro-4-nitro-thiophene-2-sulfonic acid [2-(6-chloro-1H-indol-3-yl)-ethyl]-amide [60];
5-Chloro-4-nitro-thiophene-2-sulfonic acid (4-methoxy-phenyl)-amide [61];
5-Chloro-4-nitro-thiophene-2-sulfonic acid phenylamide [62];
5-Chloro-4-nitro-thiophene-2-sulfonic acid p-tolylamide [63];
5-Chloro-4-nitro-thiophene-2-sulfonic acid benzylamide [64];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
5-Nitro-thiophene-2-sulfonic acid (4-chloro-phenyl)-amide [68];
4-Nitro-thiophene-2-sulfonic acid (4-chloro-phenyl)-amide [69];
5-Chloro-thiophene-2,4-disulfonic acid bis-[(4-chloro-phenyl)-amide] [70];
5-Ethyl-4-nitro-thiophene-2-sulfonic acid (4-chloro-phenyl)-amide [71];
Thioacetic acid S-[5-(4-chloro-phenylsulfamoyl)-3-nitro-thiophen-2-yl]ester [72];
5-Methyl-4-nitro-thiophene-2-sulfonic acid (4-chloro-phenyl)-amide [73];
5-Methyl-thiophene-2,4-disulfonic acid bis-[(4-chloro-phenyl)-amide] [74];
5-Chloro-4-nitro-thiophene-2-sulfonic acid (3-trifluoro-methyl-benzyl)-(4-trifluoromethyl-benzyl)-amide [75];
4-Nitro-thiophene-2-sulfonic acid (4-trifluoromethyl-phenyl)-amide [76];
4-Nitro-thiophene-2-sulfonic acid [2-(1H-indol-3-yl)-ethyl]-amide [77];
5-(1-Methyl-5-tri-fluoromethyl-1H-pyrazol-3-yl)-thiophene-2-sulfonic acid (3,5-bis-trifluoromethyl-phenyl)-(3-trifluoro-methyl-benzyl)-amide [78];
5-Morpholin-4-yl-4-nitro-thiophene-2-sulfonic acid (4-chloro-phenyl)-amide [79];
5-(2-Methoxy-ethylamino)-4-nitro-thiophene-2-sulfonic acid (4-chloro-phenyl)-amide [80];
4-Chloro-N-[2-(5-chloro-1H-indol-3-yl)-ethyl]-3-nitro-benzenesulfonamide [81];
N-[2-(5-Chloro-1H-indol-3-yl)-ethyl]-4-methyl-3-nitro-benzenesulfonamide [82];
N-(1H-Benzoimidazol-2-yl)-4-chloro-3-nitro-benzenesulfonamide [83];
6-Chloro-imidazo[2,1-b]thiazole-5-sulfonic acid (3,5-bis-trifluoro-methyl-phenyl)-amide [84];
2,3-Dihydro-benzo[1,4]dioxine-6-sulfonic acid (4-chloro-phenyl)-amide [85];
2,3-Dihydro-benzo[1,4]dioxine-6-sulfonic acid (3,5-bis-trifluoromethyl-phenyl)-amide [86];
6-Phenoxy-pyridine-3-sulfonic acid (4-chloro-phenyl)-amide [87];
5-Chloro-3-methyl-benzo[b]thiophene-2-sulfonic acid (4-chloro-3-nitro-phenyl)-amide [88];
N-(3,5-Bis-trifluoromethyl-phenyl)-4-pyrazol-1-yl-benzenesulfonamide [89];
4-(4-Chloro-phenylsulfamoyl)-3,5-dimethyl-1H-pyrrole-2-carboxylic acid ethyl ester [90];
4-(3,5-Bis-trifluoromethyl-phenylsulfamoyl)-3,5-dimethyl-1H-pyrrole-2-carboxylic acid [91];
4-(4-Chloro-phenylsulfamoyl)-3,5-dimethyl-1H-pyrrole-2-carboxylic acid [92];
2-(4-Chloro-phenylsulfamoyl)-4-methyl-thiazole-5-carboxylic acid ethyl ester [93];
3,5-Dichloro-N-(4-chloro-phenyl)-4-hydroxy-benzenesulfonamide [94];
N-(3,5-Bis-trifluoromethyl-phenyl)-3,5-dichloro-4-hydroxy-benzenesulfonamide [95];
3,5-Dichloro-4-hydroxy-N-(4-trifluoromethyl-phenyl)-benzenesulfonamide [96];
N-(4-Chloro-phenyl)-4-nitro-benzene-sulfonamide [97];
N-(3,5-Bis-trifluoromethyl-phenyl)-4-nitro-benzenesulfonamide [98];
4-Amino-N-(3,5-bis-trifluoromethyl-phenyl)-3-chloro-benzenesulfonamide [99];
3-Nitro-N-(4-trifluoromethyl-phenyl)-benzenesulfonamide [100];
3,5-Dichloro-N-(3,5-dichloro-phenyl)-4-hydroxy-benzenesulfonamide [101];
4-Amino-3-chloro-N-(4-chloro-phenyl)-benzenesulfonamide [102];
3-Chloro-N-(4-chloro-phenyl)-4-methoxy-benzenesulfonamide [103];
N-(3,5-Bis-trifluoromethyl-phenyl)-3-chloro-4-methoxy-benzene-sulfonamide [104];
N-(3-Chloro-4-nitro-phenyl)-3,5-bis-trifluoromethyl-benzenesulfonamide [105];
3-(4-Acetyl-piperazin-1-yl)-N-(3,5-bis-trifluoromethyl-phenyl)-4-nitro-benzenesulfonamide [106];
N-(3,5-Bis-trifluoromethyl-phenyl)-2-nitro-benzenesulfonamide [107];
3-(3,5-Bis-trifluoromethyl-phenylsulfamoyl)-benzoic acid [108];
3,5-Dichloro-N-(4-chloro-benzyl)-4-hydroxy-benzenesulfonamide [109];
3,5-Dichloro-4-hydroxy-N-(4-trifluoromethyl-benzyl)-benzenesulfonamide [110];
3,5-Dichloro-4-hydroxy-N-[2-(1H-indol-3-yl)-ethyl]-benzenesulfonamide [111];
4,5-Dibromo-thiophene-2-sulfonic acid (3,5-dichloro-phenyl)-amide [112];
N-(3,5-Dichloro-phenyl)-4-oxazol-2-yl-benzenesulfonamide [113];
4-Bromo-5-chloro-thiophene-2-sulfonic acid (3,5-bis-trifluoromethyl-phenyl)-amide [114];
4-Bromo-5-chloro-thiophene-2-sulfonic acid (3,5-dichloro-phenyl)-amide [115];
5-Benzenesulfonyl-thiophene-2-sulfonic acid (3,5-bis-trifluoromethyl-phenyl)-amide [117];
5-Benzenesulfonyl-thiophene-2-sulfonic acid (2,4-dichloro-phenyl)-amide [118];
5-Chloro-3-methyl-benzo[b]thiophene-2-sulfonic acid (3,5-bis-trifluoromethyl-phenyl)-amide [119];
Benzo[b]thiophene-2-sulfonic acid (3,5-bis-trifluoromethyl-phenyl)-amide [120];
Benzo[1,2,5]thiadiazole-5-sulfonic acid (4-chloro-phenyl)-amide [121];
Benzo[1,2,5]thiadiazole-5-sulfonic acid (3,5-bis-trifluoromethyl-phenyl)-amide [122];
Benzo[1,2,5]thiadiazole-5-sulfonic acid (4-trifluoromethyl-phenyl)-amide [123];
5-Pyridin-2-yl-thiophene-2-sulfonic acid (3,5-bis-trifluoromethyl-phenyl)-amide [124];
4,5-Dibromo-thiophene-2-sulfonic acid (4-chloro-phenyl)-amide [125];
4,5-Dibromo-thiophene-2-sulfonic acid (4-trifluoromethyl-phenyl)-amide [126];
3,5-Dichloro-N-(4-fluoro-benzyl)-4-hydroxy-benzenesulfonamide [127];
N-(3,5-Bis-trifluoromethyl-phenyl)-2,6-dichloro-benzenesulfonamide [128];
5-Chloro-4-nitro-thiophene-2-sulfonic acid (4-methoxy-2-methyl-phenyl)-amide [129];
5-(3-Hydroxy-piperidin-1-yl)-4-nitro-thiophene-2-sulfonic acid (4-chloro-phenyl)-amide [130]; and
5-Chloro-4-nitro-thiophene-2-sulfonic acid (4-nitro-phenyl)-amide [131]
or pharmaceutically acceptable salts, esters, or prodrugs thereof,

42. A compound according to claim 41 which is selected from the following:
5-Chloro-4-nitrothiophene-2-sulfonic acid (4-chlorophenyl)amide [2];
5-Chloro-4-nitrothiophene-2-sulfonic acid (4-fluorophenyl)amide [3];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
4-Chloro-3-nitro-N-(4-trifluoromethylphenyl)-benzene-sulfonamide [35];
4-Chloro-N-(4-chlorophenyl)-3-nitrobenzene-sulfonamide [38];
5-Chloro-4-nitrothiophene-2-sulfonic acid (3,5-dichlorophenyl)amide [39];
5-Chloro-4-nitrothiophene-2-sulfonic acid (3,5-difluorophenyl)amide [40];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethyl-phenyl)-amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid (4-chlorophenyl)methylamide [51];
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (4-methoxy-phenyl)-amide [61];
5-Chloro-4-nitro-thiophene-2-sulfonic acid p-tolylamide [63];
5-Chloro-4-nitro-thiophene-2-sulfonic acid benzylamide [64];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
4-Nitro-thiophene-2-sulfonic acid (4-chloro-phenyl)-amide [69];
5-Chloro-4-nitro-thiophene-2-sulfonic acid (3-trifluoro-methyl-benzyl)-(4-trifluoromethyl-benzyl)-amide [75]; and
N-(1H-Benzoimidazol-2-yl)-4-chloro-3-nitro-benzenesulfonamide [83].

43. A compound according to claim 41 which is selected from the following:
5-Chloro-4-nitrothiophene-2-sulfonic acid (4-chlorophenyl)amide [2];
5-Chloro-4-nitrothiophene-2-sulfonic acid (4-fluorophenyl)amide [3];
5-Chloro-4-nitrothiophene-2-sulfonic acid (4-hydroxyphenyl)amide [5];
5-Chloro-4-nitrothiophene-2-sulfonic acid (4-trifluoromethylphenyl)amide [6];
5-Chloro-4-nitrothiophene-2-sulfonic acid (4-fluorophenyl)methylamide [7];
5-Bromothiophene-2-sulfonic acid (3,5-bis-trifluoromethylphenyl)amide [18];
5-Benzenesulfonyl-thiophene-2-sulfonic acid (4-trifluoromethylphenyl)amide [23];
4,5-Dibromothiophene-2-sulfonic acid (3-trifluoromethylphenyl)amide [27];
4,5-Dibromothiophene-2-sulfonic acid (3,4-dichlorophenyl)amide [28];
5-Chlorothiophene-2,4-disulfonic acid bis-[(4-trifluoromethylphenyl)amide] [33];
4-Chloro-3-nitro-N-(4-trifluoromethylphenyl)benzene-sulfonamide [35];
5-Chloro-4-nitrothiophene-2-sulfonic acid (3,5-dichlorophenyl)amide [39];
5-Chloro-4-nitrothiophene-2-sulfonic acid (3,5-difluorophenyl)amide [40];
5-(1-Methyl-5-trifluoromethyl-1H-pyrazol-4-yl)-thiophene-2-sulfonic acid (3,5-bis-trifluoromethylphenyl)amide [43];
5-Chloro-4-nitrothiophene-2-sulfonic acid (3,5-bis-trifluoromethylphenyl)amide [44];
5-Chloro-4-nitrothiophene-2-sulfonic acid 4-fluorobenzylamide [45];
5-Chloro-4-nitrothiophene-2-sulfonic acid 4-trifluoromethylbenzylamide [46];
5-Chloro-4-nitrothiophene-2-sulfonic acid [2-(1H-indol-3-yl)-ethyl]amide [48];
5-Chloro-4-nitrothiophene-2-sulfonic acid [2-(1H-indol-3-yl)-1-methylethyl]amide [49];
5-Chloro-4-nitrothiophene-2-sulfonic acid methyl-(4-trifluoromethylphenyl)amide [50];
5-Chloro-4-nitrothiophene-2-sulfonic acid (4-chlorophenyl)methylamide [51]; and
5-Chloro-4-nitrothiophene-2-sulfonic acid methyl (4-trifluoromethylbenzyl)amide [52]
5-Chloro-4-nitro-thiophene-2-sulfonic acid benzyl-(4-fluoro-benzyl)-amide [53];
5-Chloro-4-nitro-thiophene-2-sulfonic acid 3,5-dichloro-benzylamide [54];
5-Chloro-4-nitro-thiophene-2-sulfonic acid 3,5-difluoro-benzylamide [55];
5-Chloro-4-nitro-thiophene-2-sulfonic acid 4-chloro-benzylamide [56];
5-Chloro-4-nitro-thiophene-2-sulfonic acid [1-(4-fluoro-phenyl)-ethyl]-amide [57];
5-Chloro-4-nitro-thiophene-2-sulfonic acid (1H-benzo-imidazol-2-yl)-amide [59];
5-Chloro-4-nitro-thiophene-2-sulfonic acid [2-(6-chloro-1H-indol-3-yl)-ethyl]-amide [60];
5-Chloro-4-nitro-thiophene-2-sulfonic acid (4-methoxy-phenyl)-amide [61];
5-Chloro-4-nitro-thiophene-2-sulfonic acid phenylamide [62];
5-Chloro-4-nitro-thiophene-2-sulfonic acid p-tolylamide [63];
5-Chloro-4-nitro-thiophene-2-sulfonic acid benzylamide [64];
5-Chloro-4-nitro-thiophene-2-sulfonic acid benzyl-(4-chloro-phenyl)-amide [65];
5-Chloro-4-nitro-thiophene-2-sulfonic acid benzyl-(4-methoxy-phenyl)-amide [66];
5-Chloro-4-nitro-thiophene-2-sulfonic acid (4-chloro-phenyl)-(3-trifluoromethyl-benzyl)-amide [67];
4-Nitro-thiophene-2-sulfonic acid (4-chloro-phenyl)-amide [69];
5-Chloro-thiophene-2,4-disulfonic acid bis-[(4-chloro-phenyl)-amide] [70];
Thioacetic acid S-[5-(4-chloro-phenylsulfamoyl)-3-nitro-thiophen-2-yl]ester [72];
5-Methyl-4-nitro-thiophene-2-sulfonic acid (4-chloro-phenyl)-amide [73];
5-Methyl-thiophene-2,4-disulfonic acid bis-[(4-chloro-phenyl)-amide] [74];
5-Chloro-4-nitro-thiophene-2-sulfonic acid (3-trifluoro-methyl-benzyl)-(4-trifluoromethyl-benzyl)-amide [75];
5-(2-Methoxy-ethylamino)-4-nitro-thiophene-2-sulfonic acid (4-chloro-phenyl)-amide [80];
N-(1H-Benzoimidazol-2-yl)-4-chloro-3-nitro-benzenesulfonamide [83];
5-Chloro-3-methyl-benzo[b]thiophene-2-sulfonic acid (4-chloro-3-nitro-phenyl)-amide [88];
N-(3,5-Bis-trifluoromethyl-phenyl)-3-chloro-4-methoxy-benzene-sulfonamide [104];
N-(3-Chloro-4-nitro-phenyl)-3,5-bis-trifluoromethyl-benzenesulfonamide [105];
4,5-Dibromo-thiophene-2-sulfonic acid (3,5-dichloro-phenyl)-amide [112];
5-Benzenesulfonyl-thiophene-2-sulfonic acid (3,5-bis-trifluoromethyl-phenyl)-amide [117];
5-Chloro-3-methyl-benzo[b]thiophene-2-sulfonic acid (3,5-bis-trifluoromethyl-phenyl)-amide [119];
Benzo[b]thiophene-2-sulfonic acid (3,5-bis-trifluoromethyl-phenyl)-amide [120];
Benzo[1,2,5]thiadiazole-5-sulfonic acid (3,5-bis-trifluoromethyl-phenyl)-amide [122];
4,5-Dibromo-thiophene-2-sulfonic acid (4-chloro-phenyl)-amide [125];
4,5-Dibromo-thiophene-2-sulfonic acid (4-trifluoromethyl-phenyl)-amide [126];
N-(3,5-Bis-trifluoromethyl-phenyl)-2,6-dichloro-benzenesulfonamide [128];
5-(3-Hydroxy-piperidin-1-yl)-4-nitro-thiophene-2-sulfonic acid (4-chloro-phenyl)-amide [130]; and
5-Chloro-4-nitro-thiophene-2-sulfonic acid (4-nitro-phenyl)-amide [131].

44. A pharmaceutical composition comprising a compound as defined in any one of claims 1, 24, 31, or 40, admixed with one or more pharmaceutically acceptable diluents, excipients or carriers.

45. A method of detecting the binding of a ligand to HDM2, said method comprising the steps of:
(i) contacting a ligand with HDM2 in the presence of a p53-derived peptide; and
(ii) detecting any change in the interaction between HDM2 and said p53-derived peptide;
and wherein said ligand is a compound as defined in any one of claims 1, 24, 31, or 40.

46. The method according to claim 45, wherein said assay is capable of identifying candidate compounds that inhibit the interaction between HDM2 and p53 and/or E2F.

47. The method according to claim 45, wherein said assay is a competitive binding assay.

48. The method according to claim 45, wherein said p53-derived peptide is a fluorescently labelled or biotinylated p53-derived peptide.

49. A combination comprising at least one compound as defined in any one of claims 1, 24, 31, or 40, and at least one cytotoxic agent.

50. A combination according to claim 49, wherein said cytotoxic agent is a chemotherapeutic agent.

51. A combination according to claim 50, wherein said chemotherapeutic agent is cisplatin or etoposide.

52. A pharmaceutical composition comprising at least one compound as defined in any one of claims 1, 24, 31, or 40, and one or more cytotoxic agents, admixed with a pharmaceutically acceptable diluent, excipient or carrier.

53. A method of treating a proliferative disorder, said method comprising administering to a subject at least one compound as defined in any one of claims 1, 24, 31, or 40, consecutively, simultaneously or sequentially with one or more other cytotoxic agents.

54. A method of treating a proliferative disorder, said method comprising administering to a subject at least one compound as defined in any one of claims 1, 24, 31, or 40, consecutively, simultaneously or sequentially with radiotherapy.
